(19) United States
(12) Patent Application Publication
(10) Pub. No.: US 2026/0263551 A1
(43) Pub. Date: Sep. 10, 2026

(54) TREATING CONDITIONS WITH HYPOGLYCEMIA ASSOCIATED WITH HYPERINSULINEMIA

(71) Applicants: RESILIUN B.V., Koekange (NL); BIOTEMPT B.V., Den Haag (NL)

(72) Inventors: Gert WENSVOORT, Koekange (NL); Eric CLAASSEN, Lelystad (NL)

(73) Assignees: RESILIUN B.V., Koekange (NL); BIOTEMPT B.V., Den Haag (NL)

(21) Appl. No.: 18/992,888

(22) PCT Filed: Jul. 12, 2023
(86) PCT No.: PCT/NL2023/050374

§ 371 Date: Jan. 9, 2025

Related U.S. Application Data
(60) Provisional application No. 63/368,214, filed on Jul. 12, 2022.

(51) Int. Cl. A61K 38/08; A61P 3/08

(52) U.S. Cl. CPC A61K 38/08; A61P 3/08

(57) ABSTRACT

The disclosure relates to modulators and/or antagonists of insulin-insulin receptor signaling complexes and methods for selecting such modulators and/or antagonists. Such modulators and/or antagonists are characterized by, for example, type 2 diabetes, obesity, hyperglycemia, hyperinsulinemia, insulin overdose, chronic kidney disease, type 1 diabetes, insulin resistance and insulin resistance. It can be used to treat a mammalian subject suffering from a disease state and condition, or to prevent the onset of a subject at risk as described above. The disclosure provides novel modulators and/or antagonists of insulin-insulin receptor signaling complexes, methods for selecting such modulators and/or antagonists, and disease states and conditions characterized by abnormally increased production and/or utilization of insulin. The use of such modulators and/or antagonists for the treatment or prevention of conditions of endogenous hyperinsulinism, preferably congenital hyperinsulinism (CHI) is also provided.

### CROSS REFERENCE TO RELATED APPLICATIONS

The present application is a § 371 National Phase application based on PCT/NL2023/050374 filed Jul. 12, 2023, which claims the benefit of U.S. Provisional Application No. 63/368,214, filed Jul. 12, 2022, the subject matter of each of which is incorporated by reference in their entirety.

### TECHNICAL FIELD

The application relates generally to medicine and the treatment of, for example, conditions associated with hyperinsulinemia. Insulin secretion-related disorders are a variety of severe conditions typically characterized by the lack of treatments and the serious burden for patients. Novel therapeutic strategies are urgently needed to solve the unmet needs typical of insulin secretion-related disorders. A promising approach relies on the use of peptides to target specific relevant pathways. C-peptide-derived sequences have shown promise to modulate insulin secretion and other processes, such as angiogenesis. The application also relates to substances and methods of treating or preventing hypoglycemia in hyperinsulinemia, in particular in the most causes of persistent hypoglycemia in congenital hyperinsulinemia in children.

### BACKGROUND

Insulin secretion-related disorders (ISRDs) are a heterogeneous group of conditions characterized by altered insulin secretion, resulting in serious debilitating conditions. ISRDs are a fast-growing global problem collectively affecting more than 6% of the world's adult population (with T1DM patients accounting for 5-15% of these numbers) and increasing numbers over the last decades. Because of the large patient population and the elevated disease burden, ISRDs pose an immense social and health economic impact globally. Insulin is central in these disorders, a peptide hormone that maintains normal blood glucose levels by facilitating cellular glucose uptake. Pancreatic B-cells are finely tuned to secrete insulin so that plasma glucose levels are maintained within a narrow physiological range (3.5-5.5 mmol/L). Hyperinsulinemic hypoglycemia (HH) is the inappropriate secretion of insulin in the presence of low plasma glucose levels and leads to severe and persistent hypoglycemia in neonates and children. Mutations in 12 different key genes (ABCC8, KCNJ11, GLUD1, GCK, HADH, SLC16A1, UCP2, HNF4A, HNF1A, HK1, PGM1 and PMM2) that are involved in the regulation of insulin secretion from pancreatic B-cells have been described to be responsible for the underlying molecular mechanisms leading to congenital HH.

When insulin is overly secreted, as for example observed in congenital hyperinsulinism (CHI), it may result in severe pathology characterized by acute hypoglycemia. Whereas deficient secretion of insulin, as in type 1 diabetes (T1DM), can lead to severe hyperglycemia. Both conditions can potentially lead to life-threatening situations. In the case of T1DM, the discovery of insulin replacement represented a major breakthrough for treatment from which millions of patients benefit to date. However, despite insulin replacement, diabetes patients still face a significant disease burden, manifested, among others by metabolic dysregulation that leads to disease-associated complications, such as vasculopathies. Insulin is the most important hormone for controlling the concentration of glucose in the blood. As food is eaten, blood glucose rises, and the pancreas secretes insulin to keep the blood glucose in the normal range. Insulin acts by driving glucose into the cells of the body. This action of insulin has two effects 1) maintaining blood glucose levels and 2) storing glucose particularly as glycogen in the liver. Once feeding is completed and the glucose levels fall, insulin secretion is turned off, allowing the stores of glucose in glycogen to be released into the bloodstream to keep blood glucose normal. In addition, with the switching off of insulin secretion, protein and fat stores become accessible and can be used instead of glucose as sources of fuel. In this manner, whether one eats or is fasting blood glucose levels remain in the normal range and the body has access to energy at all times.

In less common hyperinsulinemic ISRDs, such as CHI (Therapies and outcomes of congenital hyperinsulinism-induced hypoglycaemia. Diabet Med. 2019 January; 36 (1): 9-21), the immediate treatment goal is to stabilize plasma glucose levels and achieve normoglycaemia. This is usually achieved by additional dextrose administration. Oral dextrose is rarely able to prevent severe hypoglycaemia; in many cases high concentration dextrose is required through a central venous catheter. To increase blood glucose, glucagon promotes hepatic glucose output by increasing glycogenolysis and gluconeogenesis and by decreasing glycogenesis and glycolysis in a concerted fashion via multiple mechanisms. Children with CHI readily respond to glucagon treatment allowing de-novo glucogenesis;

continuous infusions of glucagon, administered intravenously, may reduce the dependence on large volumes of fluid, particularly if central venous catheter access is difficult. Early use of glucagon is therefore advocated to ensure rapid achievement of normoglycaemia without inducing fluid overload and complications such as pulmonary oedema, heart failure and electrolyte imbalance. Glucagon has been used in continuous subcutaneous infusions in the long-term treatment of CHI; however, catheter occlusion commonly occurs as a result of fibrillation of native glucagon in slow-moving solutions rendering treatment unsafe and ineffective. Newer glucagon formulations that are soluble and stable in saline may provide long-term treatment options. While glucagon is generally effective in CHI, side effects need to be monitored, including the possibility of necrolytic migratory erythema.

The first step in insulin action is the binding of hormones to the insulin receptor (INSR), an integral membrane glycoprotein, also called CD220 or HHF5. INSR belongs to the tyrosine kinase growth factor receptor superfamily and consists of two extracellular a subunits that bind to insulin and two transmembrane β subunits with intrinsic tyrosine kinase activity. An amino acid sequence of INSR is described in U.S. Pat. No. 4,761,371 and as NCBI reference sequence NP_000199.2. INSR is expressed as two isoforms, INSR-A and INSR-B. INSR isoforms also form INSR-A/INSR-B and hybrid INSR/IGF-1R receptor heterodimers, but their role in physiology and disease is not yet fully understood.

When insulin binds to INSR, the receptor is activated by tyrosine autophosphorylation, and INSR tyrosine kinase phosphorylates various effector molecules including insulin receptor substrate-1 (IRS-1), resulting in hormonal action (Ullrich et al., Nature 313:756-761, 1985; Goldfine et al., Endocrine Reviews 8:235-255, 1987; White and Kahn, Journal Bio26. 1-4, 1994). IRS-1 binding, and phosphorylation ultimately increases the high affinity glucose transporter (Glut4) molecule on the outer membrane of insulin responsive tissues, including muscle cells and adipose tissue, resulting in increased uptake of glucose from blood. Glut4 is transferred from intracellular vesicles to the cell surface where it can mediate transport of glucose into the cell. Increased INSR signaling leads to increased glucose uptake by cells, hypoglycemia (decreased circulating glucose), and all resulting sequelae.

Endogenous hyperinsulinemia (HI) is an abnormal clinical condition that involves excessive insulin secretion. It is related in 55% of cases to insulinoma. Other causes of endogenous hyperinsulinemia are possible such as islet cell hyperplasia, nesidioblastosis, or antibodies to insulin or to insulin receptor. Differentiation between these different etiologies may be difficult especially in cases where morphological examinations are negative. Approximately 1 in every 50,000 babies is born with the disease congenital hyperinsulinemia (CHI). A supposedly genetic defect means that the beta cells in the pancreas of these children produce too much insulin. As a result, the children have a constant risk of a low blood glucose level (hypoglycemia), which can lead to serious brain damage. Without prompt intervention, most children would die.

Congenital hyperinsulinism (Horm Res Paediatr 2018; 89:82-89) is characterized by persisting hypoglycemia due to dysregulated and excess secretion of insulin. It comprises a heterogeneous group of disorders, with the underlying genetic etiology identified in approximately 40% of the patients. Until now, 11 different causative mutations have been described. These mutations mostly seem to relate to the ATP-sensitive K⁺channel (KATP channel) that senses metabolic changes in the pancreatic β-cell, thereby coupling metabolism to electrical activity and ultimately to insulin secretion. When KATP channels open, B-cells hyperpolarize, and insulin secretion is suppressed. In CHI, opening these channels seems compromised, resulting in a continuing secretion of insulin, even in conditions of (persisting) hypoglycemia. Congenital hyperinsulinism is a rare genetic disorder-1 in 50,000 births-caused by mutations in genes responsible for producing insulin. CHI patients are characterized by abnormally high insulin secretion by the β-cells in the pancreas, which leads to suffering from frequent episodes of low blood sugar (hypoglycemia). As the brain of neonates and infants requires higher glucose consumption compared to adults, newborns are particularly vulnerable to CHI. The goal of emergency treatment for hyperinsulinemic hypoglycemia (HH) is to achieve normoglycemia immediately and keep plasma glucose levels at a safe range (>3.5 mmol/L) while the underlying etiology is identified and long-term treatment is determined.

If hypoglycemia is unresponsive to oral feeding, glucose should be administered intravenously. Patients with HH usually require a very high glucose infusion rate to achieve and maintain normoglycaemia. Glucagon may also be administered emergently to maintain adequate blood glucose levels.

Repeated episodes of hypoglycemia due to undiagnosed and/or untreated CHI in infants may translate into life-threatening complications with hypoglycemic brain injury and elevated risk of severe and permanent brain damage, often leading to irreversible neurodevelopmental disorders. Diffuse forms of CHI impact all the islets in the pancreas, while focal forms affect only a determined region. Treatment for focal forms of CHI relies on surgical excision of the affected area, while patients with diffuse forms depend on permanent glucose intake and a tightly controlled pharmacotherapy. However, side effects can be severe, and treatment is not always effective for patients. As a result, the only existing solution for these patients is surgical removal of the pancreas, which leads to a wide range of side effects, among them digestive- and hormonal-associated problems, and diabetes, which require a strict, life-long medication and overall reduced quality of life. Therefore, more effective, and less aggressive treatment strategies for CHI patients are urgently needed. Hyperinsulinism in infants is one of the most difficult problems to manage in contemporary pediatric endocrinology. Early recognition and accurate management of CHI is of major importance in order to prevent brain damage and consequent neurodevelopmental problems. Although the diagnosis can usually be achieved without difficulty, it presents the pediatrician with formidable day-to-day management problems. Despite recent advances in understanding the pathophysiology of hyperinsulinism, the neurological outcome remains poor, and there is often a choice of unsatisfactory treatments, with lifelong sequelae for the child and his or her family. In the focal form of CHI, hyper functioning pancreatic β-cells are localized in a solitary region of the pancreas, as a focal entity which can be detected by [F18]FDOPA-PET/CT. The treatment of choice for the focal form is surgical excision of the lesion. Instead, in patients with the diffuse form of CHI, the treatment of choice consists of supplemental glucose and pharmacotherapy. But even when treatment is available and on time, it is not always the perfect solution. More than three-quarters of patients do not respond to medication, making surgical removal of the full pancreas the only option. This major procedure can save their lives. However, it causes life-long diabetes and chronic digestive problems and carries the usual risk of potential complications for very young children who undergo major surgery.

Congenital hyperinsulinemia with hypoglycemia has also been known under a variety of different descriptive names, including “idiopathic” hypoglycemia of childhood, leucine sensitive hypoglycemia, neonatal insulinoma, pancreatic microadenomatosis, nesidioblastosis, persistent hyperinsulinemia hypoglycemia of infancy, and congenital hyperinsulinism. Both sporadic and familial forms of the disease are recognized, the former having an estimated incidence in Western Europe of one in 50 000 births. In isolated European communities, including parts of Finland, the disease incidence is much higher; the highest incidence is found in societies with high rates of consanguinity. In these cultures, particularly in the Arabian Peninsula, the incidence may be as high as one in 2500 births. Most infants with CHI present during the 1st postnatal days, with others during the 1st year. Rarely, older children present de novo with symptoms of hypoglycemia. However, postprandial hyperinsulinemic hypoglycemia after bariatric surgery (post-gastric bypass hypoglycemia) has been described as a new entity which is characterized by severe neuroglycopenic symptoms such as coma and seizures that can be disabling for the patient (J Clin Endocrinol Metab 2007; 92:4678-4685)

Prompt treatment of hypoglycemia due to hyperinsulinism (HI) is essential to prevent brain damage. Unlike other hypoglycemia-causing conditions in which alternative fuels, such as ketones or lactate, may be available for the brain during periods of hypoglycemia, HI prevents the production of these fuels and leaves the brain without a source of energy. Hypoglycemia can be treated by giving a fast-acting carbohydrate-containing drink by mouth or if severe, by giving glucose through the vein or by injecting glucagon. A child with a feeding tube can have glucose given through the tube. The goal of treatment is to prevent hypoglycemia while the child has a normal feeding pattern for age with a little extra safety built in, e.g., a one-year-old who normally would not eat overnight for 10-12 hours should be able to fast for at least 14-15 hours on a successful medical regimen.

Pharmacological treatment of CHI is limited in choice and is often complicated by inefficacy and adverse events. Current medications in use are not licensed in children with CHI. Guidelines for medical therapy vary between centers with most treatment decisions being based primarily on expert opinion. Recommended first-line pharmacological treatment consists of diazoxide in combination with the diuretic chlorothiazide. Diazoxide acts on the ATP sensitive K⁺ (K-ATP) channel to reduce cell depolarization, thereby reducing insulin secretion. However, not all patients respond to this treatment, particularly those with mutations in K-ATP channel genes (ABCC8, KCNJ11). Diazoxide therapy is often complicated by side effects, such as fluid retention, thrombocytopenia, hypertrichosis, and gastrointestinal dysmotility, which may lead to discontinuation of the treatment. Nifedipine, a calcium antagonist, has been prescribed in the past as a second-line treatment if children do not respond to diazoxide. However, nifedipine is rarely used in clinical practice as the clinical response to this drug is generally unsatisfactory. Continuous subcutaneous glucagon is another therapy choice and has been utilized not only in initial therapy of hypoglycemia but also in long-term treatment of CHI. However, the commercially available preparation of glucagon precipitates in slow-moving solutions within indwelling catheters causing obstruction and unreliable drug delivery. Therefore, continuous glucagon is not currently envisaged for long-term treatment of CHI.

Somatostatin is a hormone that preserves electrical stability of the pancreatic β-cell membrane and therefore inhibits the release of insulin. Octreotide, a short-acting somatostatin analogue, is commonly used as second-line treatment of CHI, in preference over nifedipine or continuous glucagon. The half-life of octreotide is relatively short at around 100 min; therefore, octreotide has to be administered by continuous intravenous infusion, frequent subcutaneous injections, or by continuous subcutaneous pump therapy. Long-term subcutaneous octreotide treatment is demanding on the patient and family; multiple daily injections of home management or pump therapy are not always feasible. Side effects of octreotide treatment include gastrointestinal dysmotility and tachyphylaxis, requiring escalating drug dosage to maintain efficacy.

There are limited randomized clinical trials of novel therapies in CHI, which may be due to several factors, including the limited number of patients worldwide and the complexity of multicentered trial organization. A first phase 3-trial with a long acting subcutaneously administered glucagon did not meet its primary endpoint. In a phase 1 clinical study, a single infusion of antibody XOMA 358 (aka RZ358) resulted in a dose-dependent reduction in insulin sensitivity in healthy adults. (MAbs. 2018 July; 10 (5): 796-802.), and results presented May 1, 2022, at the pediatric endocrines society's 2022 annual meeting showed reduction of hypoglycemia in infants >2 years of age. However, monoclonal antibodies are given intravenously (injected into a vein). The antibodies themselves are proteins, so giving them can sometimes cause an allergic or immunological reaction. Possible adverse effects can include hypersensitivity reactions, infusion reactions and lasting immunogenicity and these effects may be aggravated in infants. Further to CHI-medication that attempt to increase plasma glucose levels (dextrose, glucagon, and analogues) or that attempt to reduce insulin-secretion or release from beta-cells (diazoxide, somatostatin receptor analogues, nifedipine), currently no additional approved therapeutic venues exist except partial of full removal of the pancreas.

Congenital hyperinsulinism (CHI) is the most frequent cause of severe, persistent hypoglycemia (low glucose, sugar, in blood) in newborn babies and children. The disease is characterized by a high blood level of insulin, leading to a decrease in glucose (sugar). Recurrent and severe hypoglycemia can adversely affect brain function. Despite currently available therapies, CHI can result in serious neurological and developmental complications, including recurrent seizures, learning disabilities, and focal brain lesions. The condition is currently managed by feeding the patients regular high carbohydrate meals and treating them with medicines to reduce insulin secretion, such as diazoxide, chlorothiazide, nifedipine, glucagon and octreotide. The treatment might include surgery of the pancreas (pancreatectomy). However, these treatments are difficult because these patients require constant monitoring, and the drugs have undesirable adverse effects. Furthermore, although a rare disorder, CHI has been found to be a substantial economic burden on the NHS.

In short, there is a significant unmet medical need to develop a, preferably oral, therapy aimed at preventing hypoglycemia in HI, in particular in the most common cause of persistent hypoglycemia in children. In the words of Congenital Hyperinsulinism International (congenitalhi.org), we need to better understand congenital hyperinsulinism, identify new ways to improve the lives of those living with HI, reduce the incidence of irreversible brain damage, detect the genetic causes of HI types not currently known; and to find cures for each and every type of HI. A therapy that overrides the many and divers genetic causes of HI and directly aims at alleviating hyperinsulinemia driven hypoglycemia (aka hyperinsulinemic hypoglycemia, HH, J Clin Res Pediatr Endocrinol. 2017 Dec. 30;9 (Suppl 2): 69-87.) is preferred.

### The Invention

The invention discloses novel findings relating to the regulation of blood-glucose by malaria parasites (Plasmodium spp) during the blood-stage of the malarial infection cycle. In essence, (Binh et al., Glucose metabolism in severe malaria: minimal model analysis of the intravenous glucose tolerance test incorporating a stable glucose label. Metabolism. 1997 December; 46 (12): 1435-40.), basal plasma glucose is usually increased in uncomplicated malaria, implying insulin resistance. If the infection progresses, the risk of hypoglycemia will increase as host glucose production becomes insufficient for host/parasite demand. Said malaria data demonstrate that basal plasma glucose utilization is increased approximately 50% in severe malaria. Prevention and treatment of early hypoglycemia should be based on adequate glucose replacement. Strategies that reduce insulin secretion or effects appear to be of minor importance in malaria. White et al (Severe hypoglycemia and hyperinsulinemia in falciparum malaria. N Engl J Med. 1983 Jul. 14;309 (2): 61-6) observe that in falciparum malaria quinine-induced insulin secretion precipitates hypoglycemia, but other factors, including the large glucose requirements of the malaria parasites also contribute. Recent findings (Kumar et al., Mol Microbiol. 2021 May; 115 (5): 891-900) link nutrient sensing and gene expression in Plasmodium falciparum blood stage parasites, specifically of the merozoites populating the red-blood-cells of the infected host during the intraerythocytic developmental cycle (IDC) of the parasite. Typically, the merozoite expresses erythrocyte membrane protein 1 (PfEMP1) on the surface of infected erythrocytes, accompanied with the increased production of a set of proteins belonging to the serine rich antigen (SERA) family, such as SERA 5. During the IDC and to accommodate rapid parasite growth, parasite glucose requirements are high, necessitating manipulation of blood-glucose-levels of the host by the parasite.

A detailed analyses of blood-stage Plasmodial proteins (EMP1 and Sera 5; see example 1 herein) showed that these proteins extensively mimic long tandem-repeats of peptides carrying a specific elastin-receptor-complex (ERC) binding motif XGXXPG wherein G stands for the amino acid glycine, P for proline and X for any amino acid. Very specifically, primate-infecting Plasmodia were found to specifically express long stretches of repeats of the chemotactic domain VGVAPG while rodent-infecting Plasmodia were found to specifically express long stretches of repeats of the chemotactic domain PGAVPG. Each of these domains, as many of peptides carrying motif XGXXPG (see for example Table 2 in Heinz et al, Elastases and elastokines: elastin degradation and its significance in health and disease. Crit Rev Biochem Mol Biol. 2020 June; 55 (3): 252-273.) is capable to activate the ERC and the repetition of the motif is thought to lead to the formation of additional structures very close to a type VIIIb-turn facilitating peptide-cell interactions with the ERC if it occurs in the sequence multiple times.

Therewith, the invention discloses a peptide modulator of insulin-insulin receptor signaling affected by the ERC (for a graphic description see for example FIG. 1 in Haxho F, Alghamdi F, Neufeld R J, Szewczuk M R (2014) Novel Insulin Receptor-Signaling Platform. Int J Diabetes Clin Res 1:005) with reduced insulin-receptor signaling allowing promotion of insulin resistance through modulation of the insulin receptor (IR) for use in the treatment of a mammal, preferably a primate, more preferably human, or alternatively a rodent subject deemed having episodes of hypoglycemia associated with hyperinsulinemia. The mechanism of action of said peptide is to create insulin resistance in hyperinsulinemic patients, preferably in HH patients, and therewith reduce (GLUT4-mediated) glucose uptake and promote increased blood glucose levels. Blaise et al. have reported that chronic administration of elastin-derived-peptides (EDPs) in mice promote insulin resistance through modulation of the insulin receptor (IR) by the elastin-receptor-complex (ERC, Diabetes (2013) 62:3807-16.). In chow-fed C57BI/6J male mice, acute or chronic intravenous injections of EDPs induced hyperglycemic effects associated with glucose uptake reduction and insulin resistance in skeletal muscle, liver, and adipose tissue. Based on in vivo, in vitro, and in silico approaches, Blaise et al propose that this insulin resistance is due to interaction between the insulin receptor (IR) and the neuraminidase-1 subunit of the elastin receptor complex triggered by elastin-derived-peptides (EDPs) such as VGVAPG and κ-elastin (kE or kappa-elastin). This interplay was correlated with decreased sialic acid levels on the B-chain of the IR and reduction of IR signaling. This is the first study to demonstrate that EDPs, which mainly accumulate with aging, are involved in the insidious development of insulin resistance leading to diabetes type 2. Blaise et al., however, does not relate to treatment of hypoglycemia nor to treatment of hyperinsulinism, let alone does it relate to treatment of hyperinsulinemic hypoglycemia which is the topic of this present application.

In a preferred embodiment, the invention discloses said modulator having or provided with a chemotactic domain structure of the elastin receptor complex for use in the treatment of a primate, preferably human, subject deemed having episodes of hypoglycemia associated with hyperinsulinemia, such as seen with congenital hyperinsulinemia (CHI) and post-gastric bypass hypoglycemia, most preferably in infants demonstrating symptoms of said CHI, most preferably in infants having or repeatedly demonstrating symptoms of said CHI or in adults with post-gastric bypass hypoglycemia and presenting glucose plasma level events, of <4 mmol/L (<72 mg/dl), more preferably <3.5 mmol/L (<63 mg/dL), more preferably <3 mmol/L (<54 mg/dL), more preferably <2.5 mmol/L (<45 mg/dL), more preferably <2 mmol/L (<36 mg/dL). The invention discloses a modulator of insulin-insulin receptor signaling with reduced insulin-receptor signaling allowing promotion of insulin resistance and therewith capable of reducing insulin-mediated blood-glucose uptake by a fat- or muscle-cell of said subject, resulting in beneficial higher glucose plasma levels, therewith fending off the feared neurological pathology and other negative consequences of CHI. It is preferred that said modulator is capable of reducing insulin-induced translocation of glucose transporter 4 (GLUT4) to the plasma membrane of a fat- or muscle-cell of said subject, more preferably is capable of inducing whole-body (systemic) insulin resistance. In a preferred embodiment, the invention discloses a modulator according to the invention allowing promotion of insulin resistance, wherein said modulator is provided with a molecule carrying an elastin receptor binding motif (said motif herein also identified as chemotactic domain of elastin). It is preferred that said modulator is provided with or has a molecule carrying an elastin receptor binding motif that is at least functionally equivalent to a peptide with motif XGXXPG wherein G stands for the amino acid glycine, P for proline and X for any amino acid. In a preferred embodiment, said modulator comprises a peptide or peptidomimetic molecule carrying an elastin receptor binding motif that is at least functionally equivalent to a peptide with motif XGXXPG wherein G stands for the amino acid glycine, P for proline and X for any amino acid. It is preferred that said motif is derived from the group of primate, preferably human, elastin-derived peptides, primate, preferably human, C-peptide, or primate, preferably human, Galectin-3. In a preferred embodiment, the inventor discloses a peptide modulator according to the invention having a retro-inverso PG-domain (all-D-amino acid) peptide, such as gagpgggI, gagpggaI, agpggl, gpgggpa, gpggaI, gpggg, gpgag, gpvagp, gpavgv, that were found fit in the model of EBP designed for docking chemotactic domain VGVAPG as well.

Note that, except for glycine, all standard α-amino acids may exist in either of two optical isomers, which are the mirror image of one other; these are called L- and D-amino acids. AS used herein, when referring to an amino acid sequence of a peptide in the one letter code, lowercase letters indicate D-amino acids, whereas uppercase letters indicate L-amino acids, with the notable exception of glycine, wherein both g and/or G indicate the amino acid glycine of which no optical isomers, and thus no difference between D- or L-forms, exist. In general, all L-amino acid containing peptides are functionally equivalent to their all-D-counterparts, with the notable exception that all-D amino acids peptides are generally more resistant to common proteolytic degradation such as found in the intestinal system. As used herein, lowercase letters indicate D-amino acids, whereas uppercase letters indicate L-amino acids, with the notable exception of glycine, wherein both g and/or G indicate the amino acid glycine of which no optical isomers, and thus no difference between D- or L-forms, exist.

Also, L-amino acid peptides GGGPG and GAGPG fit the model as well. EBP-associated bioactivity is considered to depend on whether a PG-domain (having a GXXP- or pxxg-motif) peptide can adapt to a type VIII beta-turn confirmation at the proline (P/p). In one embodiment, it is preferred that said peptide with said motif is at least functionally equivalent to a peptide VGVAPGVGVAPGVGVAPG, QVGQVELGGGPGAGSLQP or GAYPGAPGAYPGAPAPGV. It is also preferred that said peptide with said motif is derived from the group of, preferably primate, preferably human, elastin-derived peptides, such as a kappa-elastin derived peptide carrying a motif XGXXPG. In a preferred embodiment and based on the modulator mimicking malaria mimicry of example 1, said peptide with said motif is at least functionally equivalent to a peptide VGVAPGVGVAPGVGVAPG or AVPGAVPGAVPG. Typically, it is disclosed to use said VGVAPG-based modulator in a primate subject, and said PGAVPG-based modulator in a rodent subject, respectively, however, for comparative purposes the reverse is provided as well. In a further embodiment said modulator comprises a peptide VGVAPG(n) or AVPG(n), wherein n indicates the number of repeat occurrences of said motif and is selected from the group of 1, 2, 3, 4, 5, 6, 7, and 8, preferably n is selected from the group of 1, 2, 3, 4, 5, 6, more preferably n is selected from the group of 1, 2, 3, 4. In another embodiment, said modulator comprises a peptide with motif gpva(n) or gpavgv(n) wherein n indicates the number of (repeat) occurrences of said motif and is selected from the group of 1, 2, 3, 4, 5, 6, 7, and 8, more preferably from 2, 3, 4, 5, 6, 7, 8, preferably n is selected from the group of 1, 2, 3, 4, 5, 6,, more preferably from 2, 3, 4, 5, 6, more preferably n is selected from the group of 1, 2, 3, 4, more preferably from 2, 3, 4. Preferably, such repeats comprise at least one tandem repeat. The invention also provides a pharmaceutical composition comprising the modulator according to the invention. In one embodiment, such a pharmaceutical composition is provided for parenteral application. In particular fast-degrading all L-amino acid peptides such as VGVAPGVGVAPGVGVAPG, AVPGAVPGAVPG, QVGQVELGGGPGAGSLQP, or GAYPGAPGAYPGAPAPGV and AQGVAPG (n=1 to 8), LQGVAPG (n=1 to 8), VGVAPG (n=1 to 8) or AVPG(n=1 to 8) with are most suited for parenteral application, wherein n indicates the number of occurrences of said motif In one other embodiment, such a pharmaceutical composition is provided for oral application. Preferred dosing requirements for parenteral application (preferably intravenous or intraperitoneal application) of such a modulator range from 1 to 1000 mg/kg, preferably from 5 to 500 mg/kg, more preferably from 10 to 100 mg/kg. In particular stabile all mirrored and D-amino acid (retro-invers) peptides such as gpavgvgpavgvgpavgv, gpvagpvagpva, pqlsgagpgggIevqgvq or vgpapagpyagpagpgpyag and gpavgqa (n=1-8), gpavgql (n=1-8), gpva (n=1-8) or gpavgv (n-1-8) are most suited for oral application. Preferred dosing requirements for oral application of such a modulator range from 1 to 10000 mg/kg, preferably from 5 to 5000 mg/kg, more preferably from 10 to 1000 mg/kg. Dosing requirements can be adjusted base on the resulting blood-glucose levels of the subject treated. The invention also discloses a method for treatment of a primate, preferably human, subject deemed having episodes of hypoglycemia associated with hyperinsulinemia comprising treating said subject with the modulator or pharmaceutical composition according to the invention.

The disclosure relates to, preferably peptide, modulator and/or antagonist of insulin-insulin receptor signaling complexes and methods for selecting such a modulator and/or antagonist. Such a modulator and/or antagonist can be used to treat a mammalian subject suffering from a disease state and condition, or to prevent the onset of disease in a subject at risk as described above. A family of facilitative glucose transporters (GLUTs) is involved in regulating tissue-specific glucose uptake and metabolism in the liver, skeletal muscle, and adipose tissue to ensure homeostatic control of blood glucose levels. Reduced glucose transport activity results in aberrant use of energy substrates and is associated with insulin resistance and type 2 diabetes. It is well established that GLUT2, the main regulator of hepatic hexose flux, and GLUT4, the workhorse in insulin- and contraction-stimulated glucose uptake in skeletal muscle, are critical contributors in the control of whole-body glycemia. Intake of carbohydrates leads to an immediate increase in circulating blood glucose levels after absorption of the glucose from the intestine. As a direct response, pancreatic beta cells sense the elevated blood glucose concentrations via a GLUT2-dependent process and increase secretion of insulin. Consequently, insulin binding to its receptors leads to enhanced glucose transport into skeletal muscle, adipose tissue, and the heart, mainly facilitated by an acute translocation of GLUT4 transporter vesicles to the plasma membrane and, in addition, to an inhibition of hepatic gluconeogenesis. Both regulatory pathways in combination result in the clearance of glucose from the bloodstream (GLUT4 exocytosis. J Cell Sci. 2011 Dec. 15;124 (Pt 24): 4147-59.). GLUT4 is an insulin-regulated glucose transporter that is responsible for insulin-regulated glucose uptake into fat and muscle cells. In the absence of insulin, GLUT4 is mainly found in intracellular vesicles referred to as GLUT4 storage vesicles (GSVs). In response to insulin stimulation, GSVs translocate to and fuse with the plasma membrane in a rapid burst and in the continued presence of insulin GLUT4 molecules are internalized and recycled back to the plasma membrane in vesicles that are distinct from GSVs and probably of endosomal origin. Not wishing to be bound to theory, insulin stimulates glucose transport into muscle and adipose tissue 10- to 30-fold with a half time of 2-5 minutes. The major glucose transporter expressed in these tissues is GLUT4. In the absence of insulin, the majority of GLUT4 is stored in small intracellular vesicles [referred to as GLUT4 storage vesicles (GSVs) or insulin responsive vesicles (IRVs)]. Following a meal, insulin is secreted by the pancreas and engages its receptor on the surface of myocytes and adipocytes, thereby activating the canonical PI3K-AKT pathway. Activation of this pathway is necessary and sufficient to trigger exocytosis of GSVs to the plasma membrane. Reduced GLUT4 trafficking is considered one of the earliest factors contributing to insulin resistance in primates, preferably humans, disruption of GLUT4 translocation to the plasma membrane in muscle or adipose tissue induces insulin resistance with reduced glucose uptake by said myocytes or adipocytes, here in the invention provided as treatment for the benefit of subjects having episodes of hypoglycemia associated with hyperinsulinemia. In particular, reduced translocation of GLUT4 in adipose tissue contributes to the development of whole-body insulin resistance.

Insulin resistance represents a state of relative unresponsiveness of peripheral tissues to react accordingly to increasing amounts of insulin in the circulation, resulting in chronically elevated blood glucose levels. The invention provides use of the phenomenon of reduced GLUT4 translocation to the plasma membrane in muscle or adipose tissue with resulting insulin resistance in a new venue to treat hyperinsulinemia driven hypoglycemia.

The disclosure provides a novel modulator and/or antagonist of insulin-insulin receptor signaling complexes, methods for selecting such modulators and/or antagonists, and disease states and conditions characterized by abnormally increased production and/or utilization of insulin. The use of such modulator and/or antagonist for the treatment or prevention of conditions of endogenous hyperinsulinism (HI), preferably congenital hyperinsulinism (CHI) is also provided.

Provided is a method for treatment of a s subject having, deemed having or suspected of having a condition with episodes of hypoglycemia associated with hyperinsulinemia (HI) comprising treating said subject with a modulator or pharmaceutical composition provided with a molecule carrying an elastin receptor binding motif at least functionally equivalent to a peptide with motif XGXXPG wherein G stands for the amino acid glycine, P for proline and X for any amino acid. Such a peptide as provided herein preferably bears a canonical XGXXPG or gpxxgx motif, allowing formation of a type VIII B-turn required for binding to its cognate receptor, the elastin receptor complex (ERC; Blanchevoye et al., J Biol Chem. 2013 Jan. 11; 288 (2): 1317-28). Herein, a condition with episodes of hypoglycemia associated with hyperinsulinemia (in shorthand hyperinsulinemic hypoglycemia) describes the condition and effects of low blood glucose caused by excessive insulin. Such a condition is biochemically often characterized by the unregulated secretion of insulin from the pancreatic β-cells in the presence of low blood glucose levels. Under normal physiological conditions, β-cells synthesize, store, and secrete insulin in a precisely controlled manner so that the fasting blood glucose level is generally kept within a narrow range of 3.5-5.5 mmol/L. As hyperinsulinemic hypoglycemia symptoms typically develop at a plasma glucose of 55 mg/dL (3.0 mmol/L) and lower in otherwise healthy individuals. At glucose levels of 55 mg/dl (3.0 mmol/L) and lower, insulin secretion is normally almost completely suppressed, detection of insulin under those circumstances is highly indicative of hyperinsulinemic hypoglycemia.

In a preferred embodiment, provided is a method for treatment of a mammal, preferably a primate, more preferably human, subject having, deemed having or suspected of having a condition with episodes of hypoglycemia associated with hyperinsulinemia (HI) and having a blood glucose level 55 mg/dl (3.0 mmol/L), preferably below a level of 50 mg/dl (2.7 mmol/L), said subject preferably also having a glucose-to-insulin ratio below 3, preferably below 2, comprising treating said subject with a modulator or pharmaceutical composition provided with a molecule carrying an elastin receptor binding motif at least functionally equivalent to a peptide with motif XGXXPG or gpxxgx wherein G/g stands for the amino acid glycine, P/p for proline and X/x for any I- or D-amino acid, respectively.

In a preferred embodiment, provided is a method for treatment of a primate, preferably human, subject having, deemed having or suspected of having a condition with episodes of hypoglycemia associated with hyperinsulinemia (HI) and having a blood glucose level 55 mg/dl (3.0 mmol/L), preferably below a level of 50 mg/dl (2.7 mmol/L), said subject preferably also having a glucose-to-insulin ratio below 3, preferably below 2, comprising treating said subject with a modulator or pharmaceutical composition provided with a molecule carrying an elastin receptor binding motif at least functionally equivalent to any of a peptide molecule is a peptide with motif selected from the group at least functionally equivalent to any of a peptide with motif VGVAPG(n), vGVAPG(n), vGvAPG(n), vGVaPG(n), vGvaPG(n), gpavgv(n),PGAVPG(n), pGvAPG(n), pGVaPG(n), pGvaPG(n), gpavgp(n), LGGGPG(n), IGGGPG(n), gpgggI(n), PGAYPG(n), pGAYPG(n), pGaYPG(n), pGAyPG(n), pGayPG(n), pgyagp(n), AQGVAPG(n), gpavgqa(n), LQGVAPG(n), or gpavgql(n), wherein n indicates the number of occurrences of said motif, wherein n varies from 1 to 8, preferably from 1 to 6, more preferably from 1 to 4, most preferably from 2 to 4, allowing targeting of the modulator to the elastin receptor on the surface of cells.

In one other embodiment, said peptide is selected from the group of VGVAPG(n), gpavgv(n), PGAVPG(n), gpvagp(n), LGGGPG(n), gpgggI(n), AQGVAPG(n), gpavgqa(n), LQGVAPG(n), gpavgql(n), PGAYPG(n) and gpyagp(n) and wherein n varies from 1 to 8, preferably from 1 to 6, more preferably from 1 to 4, most preferably from 2 to 4, allowing targeting of the modulator to the elastin receptor on the surface of cells.

In one other embodiment, said peptide is selected from the group of VGVAPG(n), gpavgv(n), PGAVPG(n), gpvagp(n), PGAYPG(n) and gpyagp(n) and wherein n varies from 1 to 8, preferably from 1 to 6, more preferably from 1 to 4, most preferably from 2 to 4, allowing targeting of the modulator to the elastin receptor on the surface of cells.

In one other embodiment, said peptide is selected from the group of VGVAPG(n), gpavgv(n), PGAVPG(n), gpvagp(n), and wherein n varies from 1 to 8, preferably from 1 to 6, more preferably from 1 to 4, most preferably from 2 to 4, allowing targeting of the modulator to the elastin receptor on the surface of cells.

In one other embodiment, said peptide is selected from the group of VGVAPG(n), gpavgv(n), PGAVPG(n) and gpvagp(n), AQGVAPG(n), gpavgqa(n), LQGVAPG(n), and gpavgql(n), wherein n varies from 1 to 8, preferably from 1 to 6, more preferably from 1 to 4, most preferably from 2 to 4, allowing targeting of the modulator to the elastin receptor on the surface of cells.

Proteins and most naturally occurring peptides are generally composed of amino acids in the L-configuration. However, D-amino acids have been detected in a variety of peptides synthesized in animal cells. Examples include opiate and antimicrobial peptides from frog skin, neuropeptides from snails, hormones from crustaceans, and venom from spiders. These D-amino acids form when L-amino acids undergo posttranslational alterations. Many proteins or peptides composed of amino acids in the L-configuration are easily degraded or tagged for selective destruction in cells or intestinal tract. Peptides that are at least partially made of D-amino acids have shown strong resistance to proteolytic degradation and have improved intestinal uptake, rending said peptides suited for oral administration. In another preferred embodiment, provided is a method for treatment of a primate, preferably human, subject having, deemed having or suspected of having a condition with episodes of hypoglycemia associated with hyperinsulinemia (HI) and having a blood glucose level 55 mg/dl (3.0 mmol/L), preferably below a level of 50 mg/dl (2.7 mmol/L), said subject preferably also having a glucose-to-insulin ratio below 3, preferably below 2, comprising treating said subject with a modulator or pharmaceutical composition provided with a molecule carrying an elastin receptor binding motif at least functionally equivalent to any of a peptide molecule is a peptide with motif selected from the group at least functionally equivalent to any of a peptide with motif gpavgv, gpavgV, gpaVgV, gpAvgV, gpAVgV, gpgggI, gpgggL, gpyagp, gpaygP, gpaYgP, gpAygP or gpAYgP. Being functional equivalent herein preferably is having functional equivalent ERC-reactivity or-bioactivity.

Lower plasma glucose levels occur in healthy individuals without symptoms or signs during extended fasting when there is use of alternative fuels such as ketones. Because of this variability there is not a single plasma glucose concentration that defines hypoglycemia. Recurrent hypoglycemia in newborns, infants, and children is not normal. Healthy newborns who experience a blood sugar level drop do not typically fall below a level of 50 mg/dl (2.7 mmol/L). Newborns and infants with blood sugars levels lower than 50 (2.7 mmol/L) or those whose blood sugar levels drop past the usual transitional newborn nadir should be evaluated and managed according to the hypoglycemia guidelines. A glucose-to-insulin ratio below 3, preferably below 2 and low concentrations of free fatty acids and ketones during hypoglycemia are highly suggestive of hyperinsulinemic hypoglycemia.

Hypoglycemia due to excess insulin is the most common type of serious hypoglycemia. It can be due to endogenous or injected insulin. Manifestations of hyperinsulinemic hypoglycemia vary by age and severity of the hypoglycemia. In general, most signs and symptoms can be attributed to (1) the effects on the brain of insufficient glucose (neuroglycopenia) or (2) to the adrenergic response of the autonomic nervous system to hypoglycemia. A few miscellaneous symptoms are harder to attribute to either of these causes. In most cases, all effects are reversed when normal glucose levels are restored.

There are uncommon cases of more persistent harm, and rarely even death due to severe hypoglycemia of this type. One reason hypoglycemia due to excessive (exogenous) insulin can be more dangerous is that insulin lowers the available amounts of most alternate brain fuels, such as ketones. Brain damage of various types ranging from stroke-like focal effects to impaired memory and thinking can occur. Children who have prolonged or recurrent hyperinsulinemic hypoglycemia in infancy can suffer harm to their brains and may be developmentally delayed. Hypoglycemia due to endogenous insulin can be congenital or acquired, apparent in the new-born period, or many years later. Hypoglycemia can be severe and life-threatening or a minor, occasional nuisance. By far the most common type of severe but transient hyperinsulinemic hypoglycemia occurs accidentally in persons with type 1 diabetes who take (too much) insulin, a condition that also can happen when persons with type 2 diabetes overdose on insulin. Persons having hyperinsulinemic hypoglycemia from excessive or overdose insulin, or overdose of drugs such that cause hyperinsulinism may often respond well to oral glucose.

There are many cases of hypoglycemia due to endogenous insulin. Foremost, congenital hyperinsulinism may be transient neonatal hyperinsulinism (mechanism not known). It may also be focal hyperinsulinism (due KATP channel disorders), such as paternal SUR1 mutation with clonal loss of heterozygosity of 11p15 or paternal Kir6.2 mutation with clonal loss of heterozygosity of 11p15. It may also manifest as diffuse hyperinsulinism due to KATP channel disorders, SUR1 mutations, Kir6.2 mutations, glukokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), and Beckwith-Wiedemann syndrome (suspected due to hyperinsulinism but pathophysiology uncertain: 11p15 mutation or IGF2 excess).

In a preferred embodiment, provided is a method for treatment of a primate, preferably human, subject having, deemed having or suspected of having a congenital HI, an ultra-rare genetic endocrine disorder that appears in 1 in 2500 to 1 in 50000 live births in various populations. Congenital H I (CHI) is characterized by excess insulin secretion, which causes repeated episodes of low blood sugar, or hypoglycemia. The condition often goes unnoticed in infants, putting them at risk of complications of recurring hypoglycemic events, including developmental delays, seizures, coma, and death.

Insulin resistance refers to a condition where a physiological amount of insulin is insufficient to obtain a normal insulin response from cells or tissues. Extreme insulin resistance is associated with type 2 diabetes, while milder insulin resistance is also associated with many disease states (such as acute inflammation or atherosclerosis) and physiological conditions (such as pregnancy or puberty) that are present in many non-type 2 diabetic individuals (Woods. et al., End, Metab & Immuno Disorders-Drug Targets 9:187-198, 2009).

### Figure Legends

FIGS. 1a and 1b xGxxPG motif preferences were detected in Plasmodial proteins related to merozoite blood-stage proteins associated with blood-stage occurrence of insulin resistance. Initial results after clustering internal dipeptide-preference at position x₁x₂ in Gx₁x₂P elastin-receptor-complex (ERC)-motif occurrences in

Plasmodial merozoite surface proteins and/or SERA5 proteins detected in
A) Plasmodial proteins derived from PRIMATE-infecting plasmodia P. vivax (n=13) or P. falciparum (n=11) as present in Uniprot database reviewed October 2022.
B) Plasmodial proteins derived from RODENT-infecting plasmodia P. yoelii yoelii (n=4) or P. berghei (n=3) as present in Uniprot database reviewed October 2022.

Out of 400 dipeptide sequences analyzed the clustering shows a preference for dipeptide VA in motif GVAP in PRIMATE-infecting Plasmodial proteins analyzed (1A) and a preference for dipeptide AV in motif GAVP in RODENT-infecting Plasmodial proteins analyzed (1B).

Rodent and primate Plasmodial protein entries in Uniprot were then probed by peptide search with various peptide motifs carrying the found GVAP and GAVP motifs. In particular GVAP was found in the VGVAPG motif that were found repeatedly present in Plasmodial proteins derived from PRIMATE-infecting plasmodia, and also found repeatedly in elastins of primates (see also tables 1 to 4).

FIG. 2

Taken from Blaise et al (Diabetes. 2013 November; 62 (11): 3807-3816. Notably: Blaise et al does not relate to treatment of hypoglycemia nor to treatment of hyperinsulinism, let alone does it relate to treatment of hyperinsulinemic hypoglycemia which is the topic of this present application). A: Blood glycemia 30 min after an intravenous single injection of various doses of kE (a collection of peptides derived from elastin and having a ERC-binding XGXXPG motif such as peptides with the bioactive motifs PGAIPG, GAVPG, GVLPG, GGVPG, and GVVPG, VGVAPG, or VVGPGA (n=7). B: Glucose uptake measured in isolated soleus muscles incubated with kE with (dark gray bars) or without insulin (black bars). White and light gray bars correspond to conditions without kE insulin at the indicated concentrations (n=5 per group). C: Time-course study of blood glucose after intravenous injection of kE once weekly compared with fed mice injected with PBS (n=10 per group). D: Food intake obtained at 1, 8, and 11 weeks (n=5 per group). E: Time-course study of blood glucose after intravenous injection of kE once weekly (n=5 per group). F: Blood glucose level in mice fasted 6 h and treated for 7 weeks with kE or PBS (n=9 per group). G: Results of glucose tolerance test (GTT) at the seventh week of injections in mice after 6-h fast (n=10 per group). H: The bar graph represents the average area under the curve (AUC) of the GTT results. I:

Glycogen quantification in mice fasted 6 h and treated for 7 weeks with kE or PBS (n=5 per group). J: Glucose 6 phosphatase (G6Pase) and phosphoenolpyruvate carboxykinase (PEPCK) expressions by quantitative real-time PCR, in liver of fasted mice treated or not with kE for 7 weeks. Target mRNA levels were normalized to 36B4 mRNA levels (n=6-9). Results are the mean±SEM. Statistically significant differences (*P<0.05, Mann-Whitney). Blood glucose conversion: mg/dl in mmol/L conversion factor: 1 mg/dl=0.0555 mmol/L

FIG. 3

Taken from WO2018/141969 and WO2018/141970. Notably: neither WO2018/141969 nor WO2018/141970 relates to treatment of hypoglycemia nor to treatment of hyperinsulinism, nor do they relate to treatment of hyperinsulinemic hypoglycemia which is the topic of this application). Various GxxP hexa-peptides were docked in the peptide-binding site of the elastin binding protein (EBP) using Vina/Autodock and PyMOL (1, 2, 3). The binding conformation of each peptide was chosen from the top 20 best scoring poses. A homology model of EBP (4) was used as receptor in the docking procedure. Peptides tested were:
VGVAPG (prototype PG-domain, GXXP-peptide ligand of EBP (4))
LGGGPG (selected from C-peptide (5))
QGQLPG (immunomodulatory peptide provided herein)
PGAYPG (selected from Galectin-3 (6))
QGVLPA (selected from loop 2 of beta-hCG (7))

Similarly, retro-inverso PG-domain (all-D-amino acid) peptide gagpgggI, gagpggaI, agpggl, gpgggpa, gpggaI, gpggg, gpgag, gpvagp, gpavgv, fit in the model of EBP designed for docking VGVAPG as well. Also, L-amino acid peptides GGGPG and GAGPG fit the model as well. EBP-associated bioactivity and thus functional equivalence is considered to depend on whether peptide with a PG-domain (having a GXXP- or pxxg-motif) can adapt to a type VIII beta-turn confirmation at the proline (P/p) (4).

### REFERENCES TO FIG. 3

1 Trott, O & Olson, AJ (2010) AutoDock Vina: improving the speed and accuracy of docking with a new scoring function, efficient optimization and multithreading, J Comp Chem 31: 455-461 .isEp
2 Seeliger, D & de Groot, BL (2010) Ligand docking and binding site analysis with PyMOL and Autodock/Vina. J Comput-Aided Mol Des 24:417-422.
3 www.pymol.org
4 Blanchevoye, C et al. (2013) Interaction between the elastin peptide VGVAPG and human elastin binding protein, J Biol Chem 288:1317-28.
5 Ido, Y et al. (1997) Prevention of vascular and neural dysfunction in diabetic rats by C-peptide Science 277:563-6.
6 De Boer, R et al. (2011) Plasma Galectin-3 Is Associated with Near-Term Rehospitalization in Heart Failure: A Meta-Analysis Journal of Cardiac Failure Vol 17, Issue 8, S93isEp
7 Khan, N A et al. (2010) Mitigation of septic shock in mice and rhesus monkeys by human chorionic gonadotrophin-related oligopeptides, Clin Exp Immunol 160:466-478.
   FIGS. 4a-4c

FIG. 4a ERC is a heterotrimeric-receptor-complex of human elastin-binding-protein (EBP), neuraminidase-1 (Neu-1) and protective-protein-Cathepsin A (PPCA). EBP consists of an alternatively spliced variant of beta-galactosidase. It binds to a hexapeptide X-Gly-X-X-Pro-Gly (XGXXPG) motif in (proteolytic fragments of) extracellular matrix proteins such as elastin and fibrillin-1. The best-known representative of the motif is hexapeptide VGVAPG found in (tropo) elastin, but many other biologically active peptides conforming to the signature sequence xGxxPG, generally called elastin peptides, have been reported as agonist. A minimally essential sequence for biological activity is GxxP, with the peptide at P adopting a type VIII beta-turn.

ERC is specifically binding to peptides with motif XGXXPG (herein also identified as GXXP-peptides, through its receptor-binding site located at EBP. The minimally essential sequence for biological activity of a peptide carrying the binding motif is GXXP, with the peptide at P adopting a type VIII beta-turn, a condition considered always met when the amino acid following the proline (at its N-terminal side) is glycine. V14 peptide VVGSPSAQDEASPL corresponding to the peptide binding site in the EPB portion of the receptor, is used to antagonize elastin peptide binding. Various galactosides, such as lactose and chondroitin sulphate can antagonize ERC-mediated signaling trough binding to the beta-galactoside-binding side or carbohydrate recognition domain (CRD) on EBP. Of note, a galactose-binding side with similar affinity for galactosugars is shared by galectins as well. Galectins are subdivided into proto-type galectins, which contain one carbohydrate recognition domain (CRD) and can form homodimers; tandem-repeat galectins that contain two distinct CRDs in tandem connected by a linker of up to 70 amino acids; and the unique chimera-type galectin-3, which consists of unusual tandem repeats of proline and glycine-rich short stretches fused onto the CRD, whereby the tandem repeats are characterized by repeat GXXP-motifs that are ligands of the ERC.

FIG. 4b Taken from Hinek et al., (Lysosomal sialidase (neuraminidase-1) is targeted to the cell surface in a multiprotein complex that facilitates elastic fiber assembly. J Biol Chem. 2006 Feb. 10;281 (6): 3698-710, incorporated herein by reference. To be clear: Hinek et al does not relate to treatment of hypoglycemia nor to treatment of hyperinsulinism, let alone does it relate to treatment of hyperinsulinemic hypoglycemia which is the topic of this present application). Upper panel, representative micrograph of cultured ASMCs immunostained with anti-tropoelastin antibody illustrating three stages of elastogenesis: accumulation of tropoelastin in the endosomes and Golgi apparatus (A), transportation in small secretory vesicles (B), and the initial assembly on a microfibrillar scaffold that occurs in the grooves on the cell surface (C). Lower panel, the proposed model of elastogenesis in which all three subunits of the cell surface-targeted multiprotein complex (S-gal/EBP, Neu1, and PPCA) act in concert in the process of extracellular assembly of elastic fibers. In the Initial Secretory Pathway, the major component of this multiprotein complex binds to XGXXPG motifs in tropoelastin in the endoplasmic reticulum (ER), and then the entire complex is transported to the Golgi compartments and to the cell surface, where the actions of two other components of this complex assure the proper release of tropoelastin from its chaperone. During Assembly of Tropoelastin onto the Microfibrillar Scaffold, Neu1 (activated by PPCA) removes terminal sialic acid residues from carbohydrate chains protruding from microfibrillar glycoproteins. This causes unmasking of penultimate galactosugars, which in turn interact with the galactolectin site of S-gal/EBP and induce release of the transported tropoelastin molecule from its chaperone to the close proximity of the acceptor sites on the microfibrillar scaffold. This guarantees the proper assembly and mutual orientation of the multiple tropoelastin molecules that are a prerequisite for their ultimate cross-linking (by lysyl oxidase) into the insoluble polymerized elastin. In the Recycling Secretory Pathway, S-gal/EBP molecules that discharge their tropoelastin cargo and temporarily depart from the cell membrane return to the endosomal compartments after their reattachment to the cell membrane-anchored Neu1.PPCA complex. Remnant proteolytic fragments of elastin peptides with XGXXPG motifs may return bound to the ERC. Once in the recycling endosome, this molecular complex released bound XGXXPG fragments, binds again to the new tropoelastin molecules delivered from the endoplasmic reticulum and chaperones them to the cell surface. MAGP, microfibril-associated glycoproteins.

FIG. 4c Cell-surface EBP activities: XGXXPG peptides bind to EBP (Blanchevoy) and initiate endocytosis of the full ERC-complex (Hinek et al., ibid), thus also at least temporarily internalising Neu-1 and PPCA, and therewith reducing the normal physiological surface activities of Neu-1 and PPCA. That in itself may be a normal aspect of vascular repair. However, in chronic excess of circulating XGXXPG peptide fragments such as fragments from circulating elastin-derived peptides, C-peptide and Galectin-3 peptides, internalising Neu-1 and PPCA with XGXXPG-bound EBP, and thus reducing cell-surface associated Neu-1 and PPCA activities, leads to cause various hallmarks of metabolic syndrome, as insulin resistance, increased LDL, vascular cell proliferation with intima thickening, and vasoconstriction with elevated blood pressure (hypertension).

Important cell-surface Neu-1 activities are inhibited by ERC internalisation: GXXP-peptide binding to ERC and subsequent internalization of ERC lead to subsequent reduction of Neu-1 directed effects on receptor maturation that may result in hallmarks of human vascular disease in metabolic syndrome: increased insulin resistance due to an inactive insulin receptor, of reduced LDL-uptake and increased LDL-blood levels due to an inactive LDL-receptor, and increased endothelial proliferation with intima thickening due to an active growth factor receptor.

At the cell-surface, Neu-1 is involved in desialylation of cell surface receptors and therewith regulates cell-surface receptor signaling (Pshezhetsky A V, Ashmarina L I. Desialylation of surface receptors as a new dimension in cell signaling. Biochemistry (Mosc). 2013 July; 78 (7): 736-45), and increases among others insulin-sensitivity and LDL-uptake, while it decreases growth-factor-receptor induced proliferation through sialic acid removal of various growth-factor receptors.

Neu-1 regulates among others insulin receptor maturation and activation (Alghamdi et al., A novel insulin receptor-signaling platform and its link to insulin resistance and type 2 diabetes. Cell Signal. 2014 June; 26 (6): 1355-68). The inhibitor of NEU1, DANA blocks both interaction between the subunits and activation of the insulin receptor (Zhang et al., Lipids Health Dis. 2019; 18:173.).

As integral membrane glycoproteins, LDL receptors also bear terminal sialic acid residues. A significant inhibition in LDL internalization of human fibroblasts was observed after neuraminidase treatment of those receptors, which was associated with a decrease in the number of active cell surface LDL receptor sites (Sprague et al., Stimulation of receptor-mediated low density lipoprotein endocytosis in neuraminidase-treated cultured bovine aortic endothelial cells. J Cell Physiol. 1988 November; 137 (2): 251-62.). Furthermore, binding of EDP to the ERC was found to modulate CD36 sialylation level and regulates oxidized LDL uptake through Neu-1 (Kaweckie et al., Identification of CD36 as a new interaction partner of membrane NEU1: potential implication in the pro-atherogenic effects of the elastin receptor complex. Cell Mol Life Sci. 2019 February; 76 (4): 791-807).

As to growth factor activities, NEU-1 induces growth factor receptor inactivation (Hinek et al., Neuraminidase-1, a subunit of the cell surface elastin receptor, desialylates and functionally inactivates adjacent receptors interacting with the mitogenic growth factors PDGF-BB and IGF-2. Am J Pathol. 2008 October; 173 (4): 1042-56).

In short, GXXP-peptide binding and subsequent Neu-1 directed effects on receptor maturation may result in increased insulin resistance due to an inactivated insulin receptor, of reduced LDL-uptake and increased LDL-blood levels due to an inactivated LDL-receptor and increased endothelial proliferation due to an as yet not inactivated (desialylated) growth-factor receptor. It is further known (Lee et al., NEU1 sialidase regulates the sialylation state of CD31 and disrupts CD31-driven capillary-like tube formation in human lung microvascular endothelia. J Biol Chem. 2014 Mar. 28;289 (13): 9121-35) that active desialylated NEU-1 inhibits angiogenesis and that angiogenic signaling may be enhanced when NEU-1 activities are blocked by treatment with DANA (as reported herein under FIG. 4).

Other cell-surface PPCA activities are inhibited by ERC internalisation: GXXP-peptide binding to ERC and subsequent internalisation of ERC lead to subsequent reduction of PPCA directed effects on proteolysis of circulating vasoconstrictive peptides, leading to more circulating vasoconstrictive peptides and therewith to elevated blood pressure, another hall mark of metabolic syndrome.

Cell-surface expressed PPCA regulates blood-pressure through proteolysis of bioactive peptides such as angiotensin (Timur et al., Lysosomal Cathepsin A Plays a Significant Role in the Processing of Endogenous Bioactive Peptides. Front Mol Biosci. 2016 Oct. 25; 3:68) and endothelin-1 (Seyrantepe et al., Enzymatic activity of lysosomal carboxypeptidase (cathepsin) A is required for proper elastic fiber formation and inactivation of endothelin-1. Circulation. 2008 Apr. 15;117 (15): 1973-81).

### FIG. 5

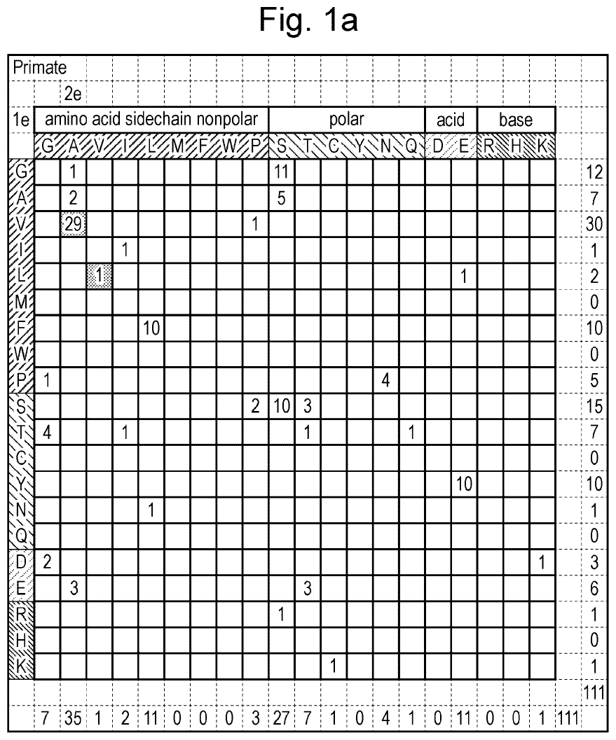

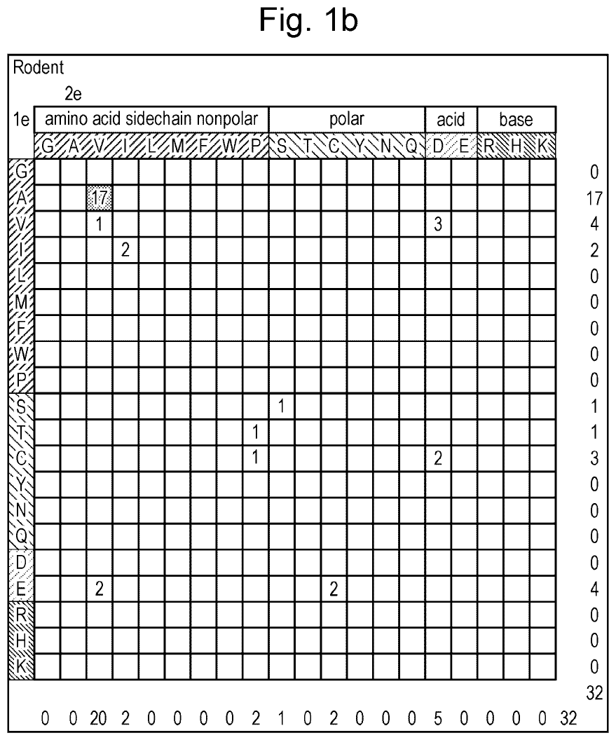

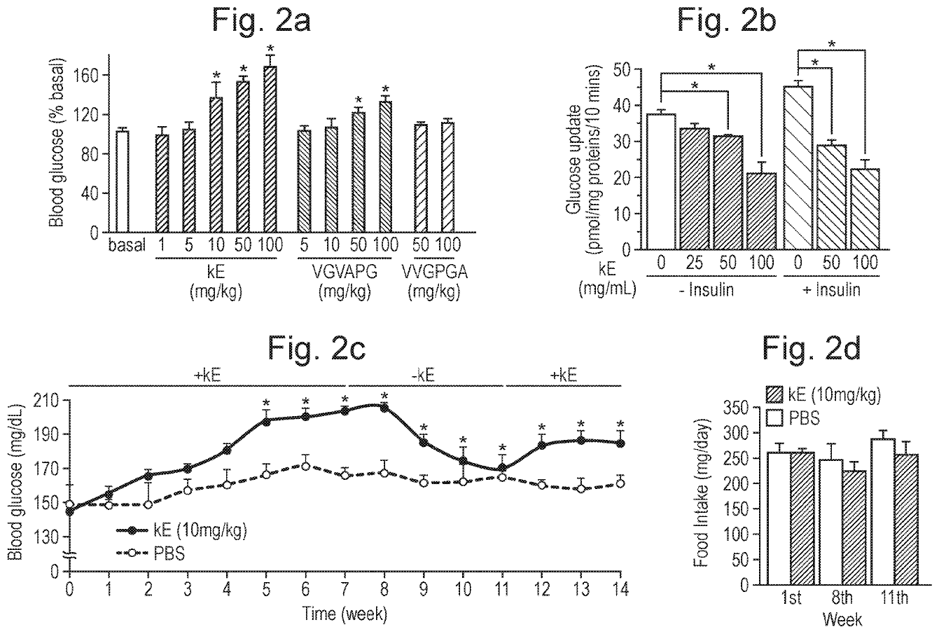

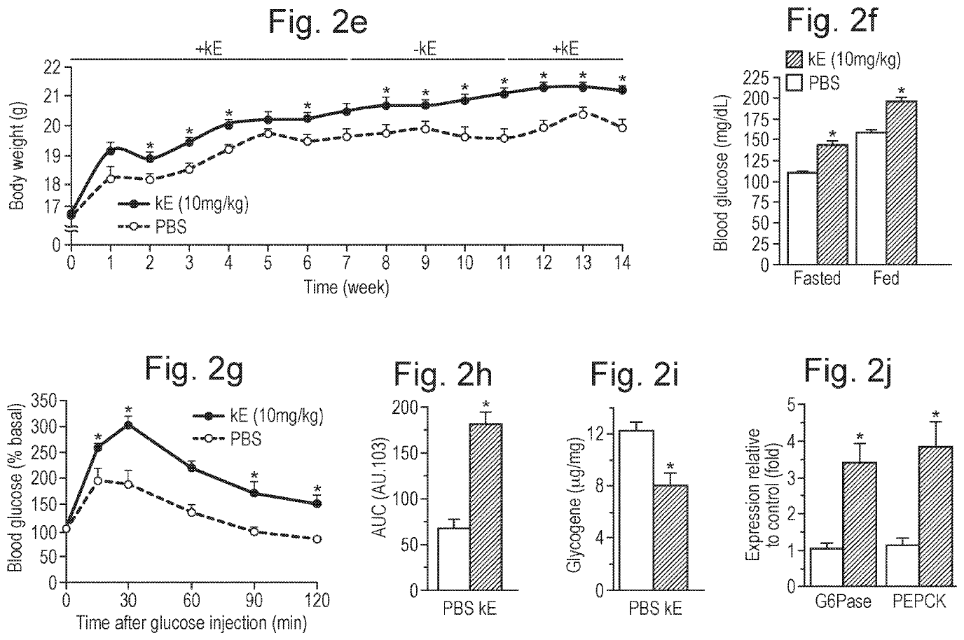

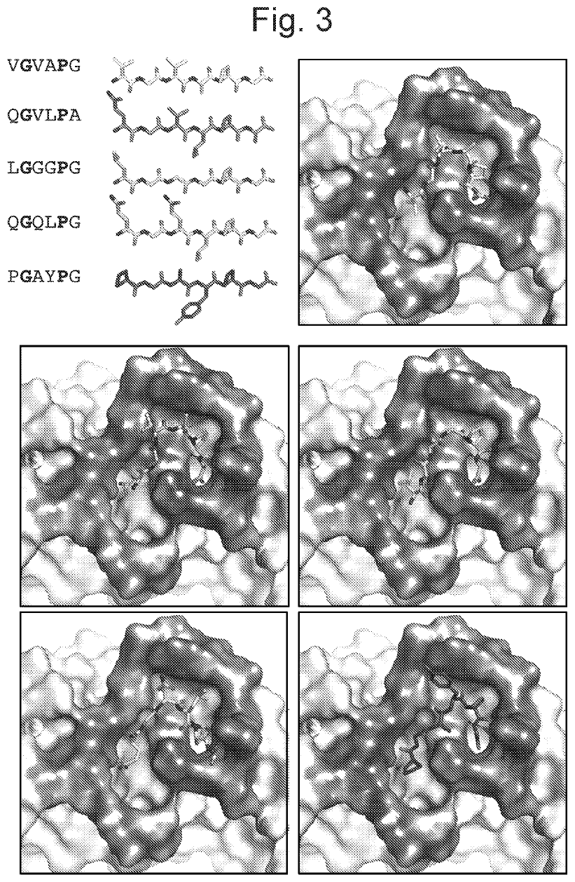

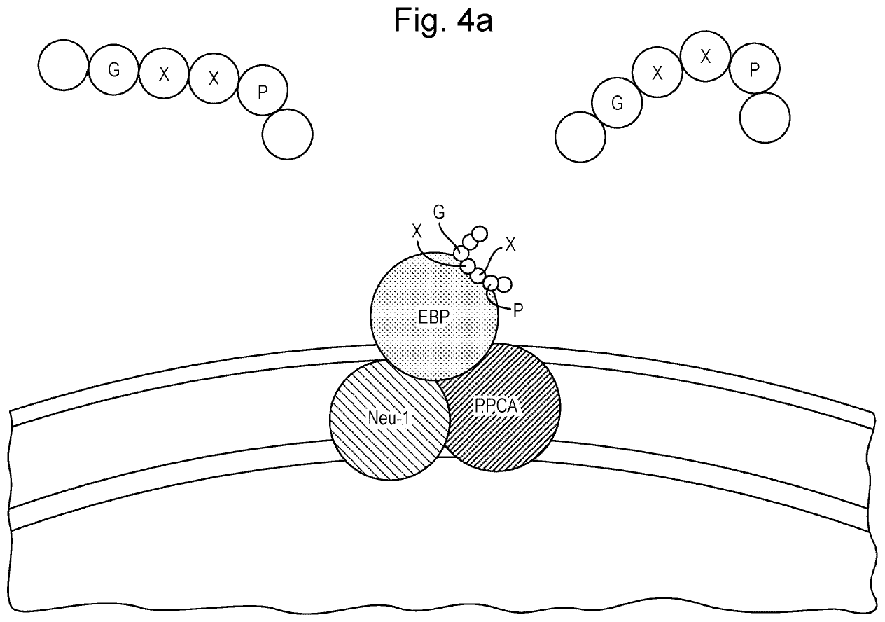

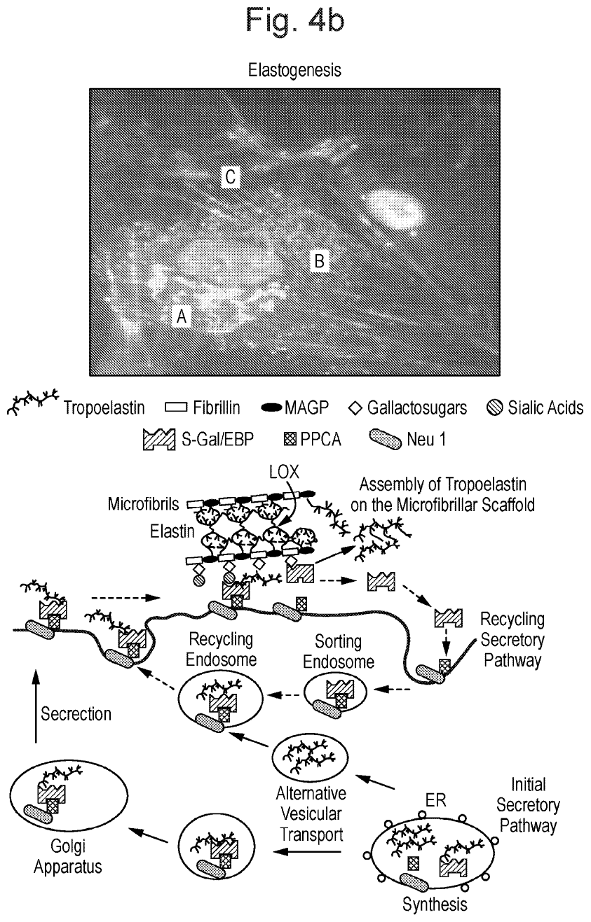

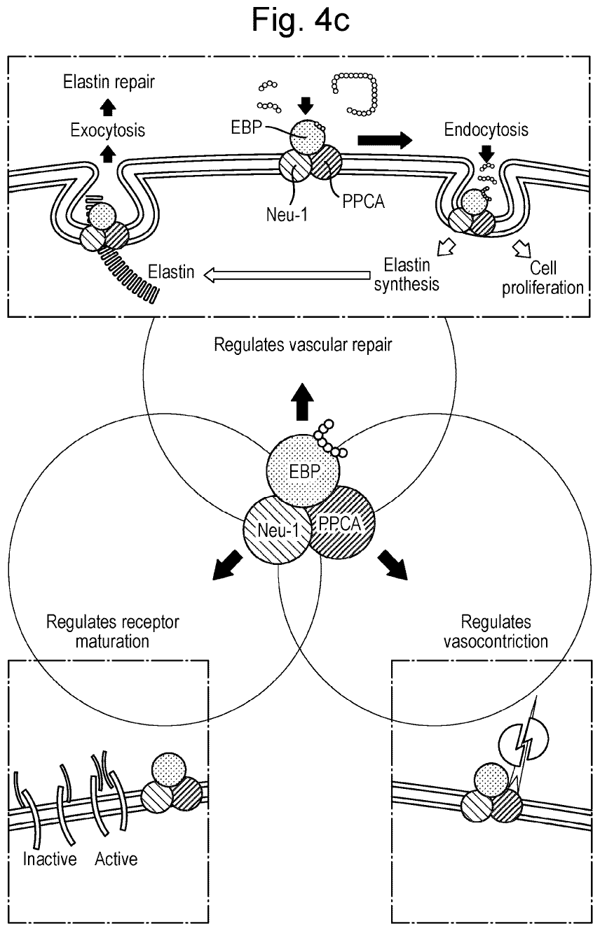

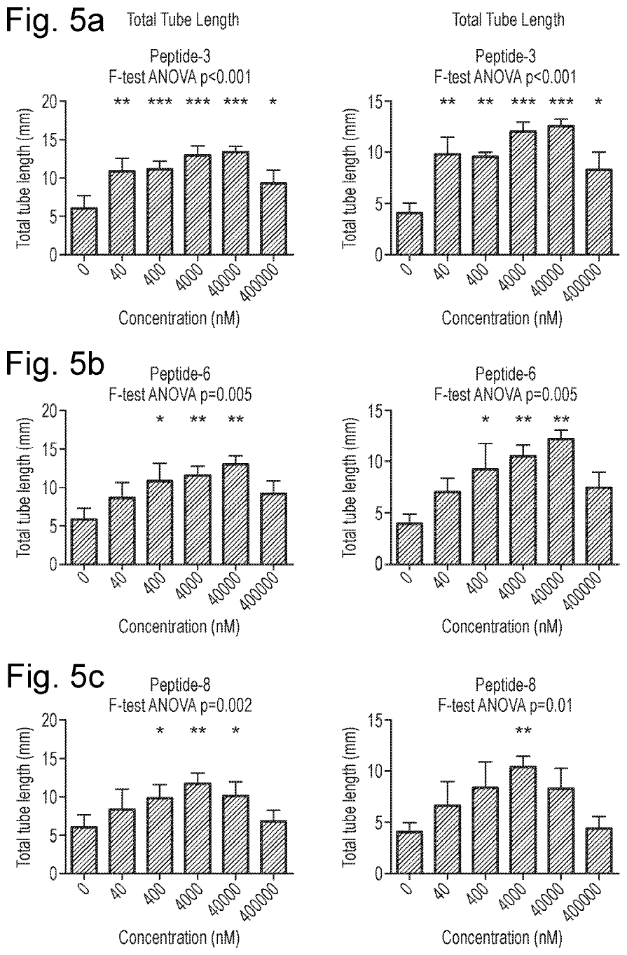

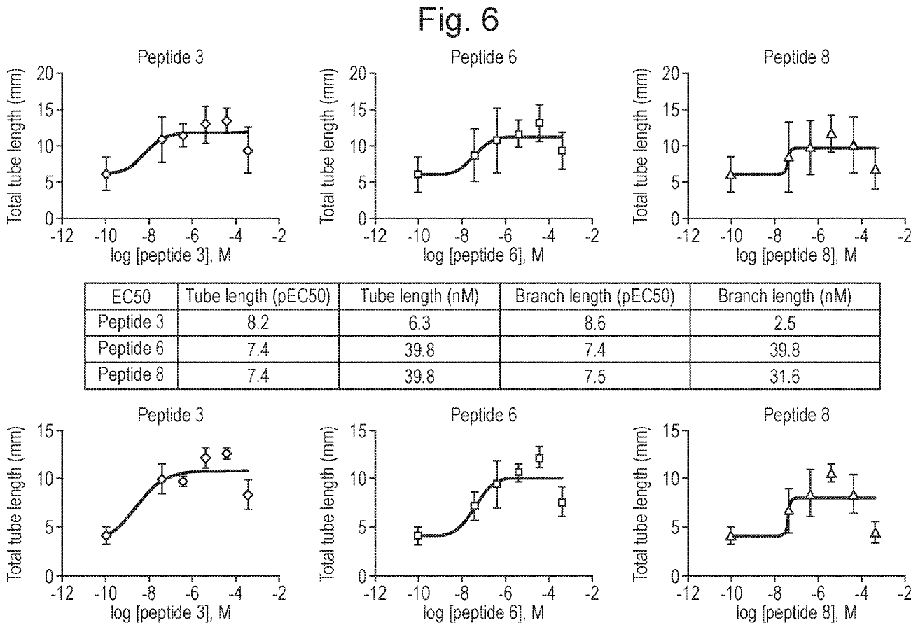

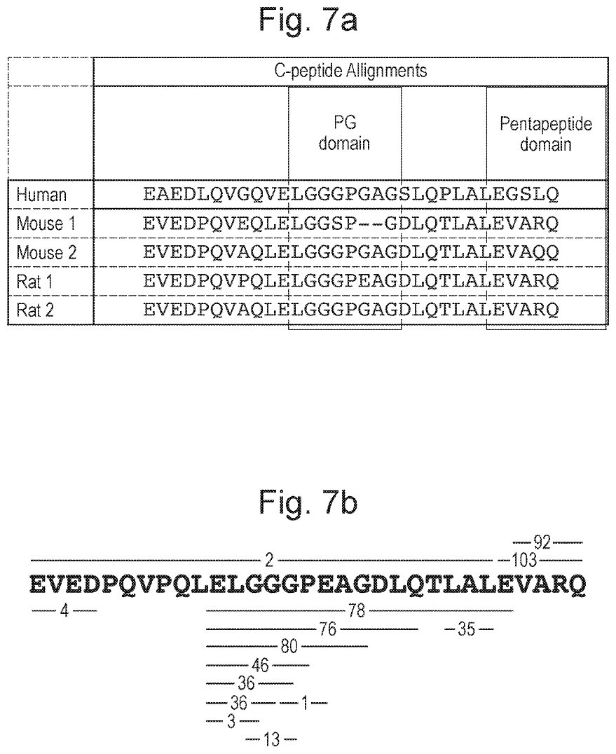

Angiogenesis—the formation of capillaries from pre-existing micro-vessels—is important in many processes. Together with inducing elevated blood glucose levels (see FIG. 1), activation of the elastin receptor complex (ERC) accelerates angiogenesis (J Cell Sci. 2005 Jan. 15;118 (Pt 2): 343-56.), whereby elevated blood glucose stimulates vascular endothelial growth factor (VEGF) accommodating said angiogenesis.

Microvascular angiogenesis assay of human pulmonary microvascular endothelial cells grown in Matrigel as determinant of ERC-reactivity and in response to increasing concentrations of GXXP-peptides 3, 6 and 8, (VGVAPGVGVAPGVGVAPG, QVGQVELGGGPGAGSLQP and GAYPGAPGAYPGAPAPGV, taken from the exon 24 region of human elastin, from the mid-portion of human C-peptide, and from the N-terminal portion of human Galectin-3, FIGS. 4a, 4b and 4c, respectively). Data shown are total tube length measurements and total branch length measurements at t=24 h. * p<0.05; ** p<0.01; *** p<0.001. At 4 h no angiogenic activity of the various peptides was as yet observed. Also, as expected, DANA significantly potentiated the angiogenic effects of peptide 3, at t=16, 24 and 40h (P=0.48, 0.011 and 0.032, respectively).

### FIG. 6

EC50 plots with pharmacological profiles of peptide 3, 6 and 8 as tested in assay of FIG. 4, showing functionally equivalent ERC-reactivity profiles of peptides 3, 6 and 8.

### FIGS. 7a and 7b

### FIG. 7a

Alignments of C-peptide amino acid sequences of human mouse and rat and localization of PG-domain and pentapeptide-domain therein.

### FIG. 7b

Taken from Ohtomo et al., (Differential effects of proinsulin C-peptide fragments on Na⁺, K⁺-ATPase activity of renal tubule segments. Diabetologia. 1998 March; 41 (3): 287-91. doi: 10.1007/s001250050905. PMID: 9541168, incorporated herein by reference. To be clear: Ohtomo et al does not relate to treatment of hypoglycemia nor to treatment of hyperinsulinism, let alone does it relate to treatment of hyperinsulinemic hypoglycemia which is the topic of this present application as described among others below and above).

Glucose metabolism increases the ATP: ADP ratio and initiates the β-cell stimulus-secretion coupling by closing ATP-regulated potassium (K⁺ATP) 1channels. These channels are the main regulators of the β-cell resting membrane potential, and their closure will initiate membrane depolarization, opening of voltage-gated L-type Ca²⁺channels, and thereby an increase in cytoplasmic free Ca²⁺ concentration ([Ca²⁺];) and exocytosis of insulin (Ashcroft F M, Harrison D E, Ashcroft S J. Glucose induces closure of single potassium channels in isolated rat pancreatic beta-cells. Nature. 1984 Nov. 29-December 5;312 (5993): 446-8. doi: 10.1038/312446a0. PMID: 6095103.). When K ATP channels open, beta-cells hyperpolarize and insulin secretion is suppressed (Koster J C, Permutt M A, Nichols C G. Diabetes and insulin secretion: the ATP-sensitive K⁺channel (K ATP) connection. Diabetes. 2005 November; 54 (11): 3065-72. doi: 10.2337/diabetes. 54.11.3065. PMID: 16249427.).

The Na⁺, K⁺-ATPase is involved in maintaining the Na⁺ and K⁺gradients across the β-cell plasma membrane. It extrudes three Na⁺ ions in exchange for two K⁺ ions, generating a net outward flow of cations through the cell membrane. This makes the pump electrogenic and results in a hyperpolarizing effect on membrane potential. Consequently, inhibition of Na⁺, K⁺-ATPase activity (for example, by ouabain) leads to β-cell membrane depolarization and Ca²⁺influx. It has therefore been postulated that a decrease in Na⁺, K⁺-ATPase-mediated ion gradients may be a contributing mechanism to insulin secretion (Owada et al., Glucose decreases Na⁺,K⁺-ATPase activity in pancreatic beta-cells. An effect mediated via Ca²⁺-independent phospholipase A2 and protein kinase C-dependent phosphorylation of the alpha-subunit. J Biol Chem. 1999 Jan. 22;274 (4): 2000-8. doi: 10.1074/jbc.274.4.2000. PMID: 9890957.).

Thus, the ATP-sensitive K⁺channel (K ATP channel) as well as the Na⁺K⁺ATPase channel may sense metabolic changes in the pancreatic beta-cell, thereby coupling metabolism to electrical activity and ultimately to modulating insulin secretion. It is herein postulated that PG-domain XGXXPG- or gpxxgx-peptides, and mixed D-/L-amino acid variants thereof, such as derived from C-peptide, elastin-peptide, Galectin-3, and in particular fragments thereof may contribute to activation of Na⁺K⁺ATPase channel activity, and therewith restore β-cell membrane polarization and help inhibit insulin secretion, following observations made earlier by Othomo et al., who observed stimulation of Na⁺, K⁺-ATPase activity in rat renal tubule segments by C-peptide (and fragments).

The amino acid sequence of rat C-peptide 1 is indicated in FIGS. 6a and 6b. It was found that full length C-peptide caused Na⁺, K⁺-ATPase activity as tested in rat renal tubule segments. Full length C-peptide activity, as tested at 5×10±7 mol/l. was set at 100%. Numbers in FIG. 6b denote the percentage of the entire molecule's stimulating activity that is retained by the respective fragments as indicated by lines. All peptide fragments were tested at 5×10±7 mol/l.

The possibility that the C-peptide fragments such as PG-domain or pentapeptide domain fragments might exert a direct stimulation of Na⁺, K⁺-ATPase as distinct from a membrane-mediated intracellular activation was also examined by Othomo et al. A purified preparation of Na⁺, K⁺-ATPase was incubated with rat C-peptide 1, and its pentapeptide domain fragments EVARQ or PG-domain peptide ELGGGPEAG. No measurable activation was found with any of these peptides with isolated

ATPase, demonstrating that the effect is not likely to be exerted on the ATPase directly, but indirectly and presumably via a receptor.

Zhong et al., (C-peptide stimulates Nat, K⁺-ATPase via activation of ERK1/2 MAP kinases in human renal tubular cells. CMLS, Cell. Mol. Life Sci. 61, 2782-2790 (2004).) determined the molecular mechanism by which C-peptide stimulates Na⁺, K⁺-ATPase in primary human renal tubular cells (HRTCs). Incubation of the cells with 5 nM human C-peptide at 37° C. for 10 min stimulated 86Rb+ uptake by 40% (p<0.01). The carboxy-terminal human pentapeptide EGSLQ was found to elicit 57% of the activity of the intact molecule. In parallel with ouabain-sensitive 86Rb+ uptake, C-peptide increased a subunit phosphorylation and basolateral membrane (BLM) abundance of the Nat, K⁺-ATPase d1 and B1 subunits. The increase in BLM abundance of the Na⁺, K⁺-ATPase α₁ and β₁ subunits was accompanied by depletion of α₁ and β₁ subunits from the endosomal compartments. C-peptide action on Na⁺, K⁺-ATPase was ERK1/2-dependent in HRTCs. C-peptide-stimulated Na⁺, K⁺-ATPase activation, phosphorylation of α₁-subunit and translocation of 1 and 1 subunits to the BLM were abolished by a MEK1/2 inhibitor (20 μM PD98059). C-peptide stimulation of 86Rb+ uptake was also abolished by preincubation of HRTCs with an inhibitor of PKC (1 μM G F109203X). C-peptide stimulated phosphorylation of human Na⁺, K⁺-ATPase a subunit on Thr-Pro amino acid motifs, which form specific ERK substrates. In conclusion, C-peptide and its pentapeptide EGSLQ stimulate sodium pump activity via ERK1/2-induced phosphorylation of Thr residues on the a subunit of Na⁺, K⁺-ATPase.

As C-peptide and some of its fragments are shown to be a ligand of the ERC, it is herein disclosed that ERC-mediated activation of Na⁺,K⁺-ATPase by C-peptide and related PG-domain or pentapeptide domain, or related peptides having both PG-domain and pentapeptide domain fragments, provide a feedback mechanism to reduce insulin exocytosis and insulin secretion of beta-cells.

### DETAILED DESCRIPTION

### Example 1

### Employing Existing Plasmodial Molecular Mimicry Patterns to Identify Essential Host Receptor Binding Motifs in Silico.

Here the invention discloses an in silico bioinformatic approach of the malaria proteome, of various blood-stage proteins encoded by multigene families, using publicly available tools and databases, to identify the best-fitting amino acid motif of the human elastin receptor-complex (ERC), that currently resists functional expression and molecular characterization studies. In essence, the invention discloses a method for identifying an amino acid motif that binds to a cellular receptor of a mammal (such a motif herein also identified as chemotactic domain), wherein the method comprises the following steps in consecutive order:
(i) selecting candidate amino acid motifs;
(ii) determining the frequency of occurrence of each of the candidate amino acid motifs in the proteome of a pathogen; wherein the pathogen is a pathogen that can infect the mammal;
and
iii) identifying the candidate amino acid motif with the highest frequency of occurrence in the proteome of the pathogen as the amino acid motif that binds to the cellular receptor of the mammal.

ERC's binding motif resides in short peptide motif with sequence xGxxPx, preferably xGxxPG, and its prototype motif VGVAPG is here found repeatedly expressed in primate-infecting Plasmodium species. These sequences have distinct vascular pathogenicity in humans. The invention discloses cluster-maps of the internal dipeptide, requiring in silico testing of 400 dipeptides contained in xGxxPG, to illustrate best fit to ERC, as found on Plasmodium blood-stage proteins.

### BACKGROUND

Acquisition of host-like proteins, and thus the ability to molecularly mimic their function, has been observed in many bacterial and viral pathogens (Cell. 2006 Feb. 24;124 (4): 767-82). In parasitic protozoa there are cases where stretches of amino acids present on a parasite-encoded cell surface protein match to regions of host proteins (Mol Biochem Parasitol. 1998 Aug. 1;94 (2): 185-96). In these and other cases, the matches correspond to a common amino acid repeat that is shared between them (Nature. 1988 Sep. 1;335 (6185): 82-5; Mol Biochem Parasitol. 1992 July; 53 (1-2): 105-12). Malaria parasites (Plasmodium spp.) invade both liver cells and red blood cells (RBC) in the vertebrate host. Typically, Plasmodium spp. varies the expression profile of its genes depending on the host it resides in and its developmental stage. In the so-called blood-stage, they actively remodel the infected RBC (iRBC) by exporting and trafficking various proteins encoded by multigene families to the RBC plasma membrane (PLOS Pathog. 2016 Nov. 16;12 (11): e1005917).

Such proteins are known to have a potential immunomodulatory role either as functional homologues of host molecules or by binding to host antigen-presenting cells (Proc Natl Acad Sci USA. 2001 Sep. 11;98 (19): 10829-32; Nature. 1999 Jul. 1;400 (6739): 73). Paine et al. (Nature. 2008 Oct. 9;455 (7214): 799-803), provide a first observation of its kind in a malaria protein that shows acquisition of host peptide sequences that are likely to be on the infected cell surface and thus may interact with the host. Considering the wildly diverse genomic repertoire of Plasmodium spp. (Genes (Basel). 2021 May 30;12 (6): 843), it is likely that many more of such acquired host-like protein sequences may be found. Moreover, considering that distinct Plasmodium spp. have had millions of years to co-evolve with their respectively preferred host, it is likely that the minimally essential as well-as the best-fitting sequences for binding to distinct host-receptors of said preferred host are enriched in said repertoire. Studying that repertoire for the preferred xGxxPG motifs in primate-infecting Plasmodia would elucidate human ERC-preferences. Control studies with rodent-infecting Plasmodia would necessarily result in a different outcome, rodents having a truncated elastin-binding protein in their ERC (unpublished). Mapping of elastin-receptor-complex preferred agonist peptides related to insulin resistance.

Results xGxxPG motif preferences were detected in Plasmodial proteins related to merozoite blood-stage proteins associated with blood-stage occurrence of insulin resistance. Initial results after clustering internal dipeptide-preference at position x₁x₂ in Gx₁x₂P elastin-receptor-complex (ERC)-motif occurrences in Plasmodial merozoite surface proteins and/or SERA5 proteins detected in
A) Plasmodial proteins derived from PRIMATE-infecting plasmodia P. vivax (n=13) or P. falciparum (n=11) as present in Uniprot database reviewed October 2022.
B) Plasmodial proteins derived from RODENT-infecting plasmodia P. yoelii yoelii (n=4) or P. berghei (n=3) as present in Uniprot database reviewed October 2022.

As shown in FIG. 1, out of 400 dipeptide sequences analyzed the clustering shows a preference for dipeptide VA in motif GVAP in the preferred chemotactic domain of binding to the ERC in PRIMATE-infecting Plasmodial proteins analyzed (1A) and a preference for dipeptide AV in motif GAVP in RODENT-infecting Plasmodial proteins analyzed (1B).

Rodent-infecting- and primate-infecting Plasmodial protein entries in Uniprot were then probed by peptide search with various peptide motifs carrying the found GVAP and GAVP motifs. In particular GVAP was found in the VGVAPG motifs that were found repeatedly present in Plasmodial proteins derived from PRIMATE-infecting plasmodia, and also found repeatedly in elastins of primates (see also tables 1 and 2).

TABLE 1

| | |
|---|---|
| Human elastin | GLVPGVGVAPGVGVAPGVGVAPGVGLAPGVGVAPGVGVAPGVGVAPGIGPGGV |
| exon 24 | |
| Rhesus monkey | GLVPGVGVAPGVGVAPGVGVAPGVGVAPGVGVAPGVGVAPGVGVAPGVGIGPGG |
| elastin exon 24 | VAGVG |
| Mouse elastin | GLGPGVGGVPGGVGVGGIPGGVGVGGVPGGVGPGGVTGIGAGPGGLGGAG |
| exon 24 | |
| Rat CDR elastin | GLGPGVGGVPGGVGVGGLPGGVGPGGVTGIGTGPGTGLVPGDLGGAG |
| exon 24 | |
| Human elastin | not expressed |
| exon 22 | |
| Rhesus monkey | not expressed |
| elastin exon 22 | |
| Mouse elastin | GAGGAGALGGLVPGAVPGALPGAVPAVPGAGGVPGAGTP |
| exon 22 | |
| Rat CDR elastin | GAGGAGTLGGLVPGAVPGALPGAVPGALPGAVPGALPGAVPGVPGTGGVPGAGT |
| exon 22 | |

The VGVAPG peptide is typically considered the prototype ERC-binding peptide and commercially available 5 (Bachem product number 4010536; described as: Chemotactic Domain of Elastin; VGVAPG stimulated human skin fibroblast proliferation and was chemotactic for fibroblasts and monocytes. The palmitoylated form is marketed as a cosmetic ingredient).

The VGVAPG peptide domain is encoded for and expressed by exon 24 of primate elastins at large. In 10 contrast, the exon 24 of rodent elastins does not encode nor expresses VGVAPG (see Table 1), and moreover typically lacks the (multiple) presence of the alanine (A) and the preferred clustered peptide with motif GAVP as shown in FIG. 1B. Several xGxxPG peptide motifs were found in the mouse exon 24 sequence that together were found in 1143 Uniprot entries, of which 420 elastin protein entries (from primates to rodents, and from fish and reptiles to birds), and in 24 Plasmodium protein entries, however none of those 24 were rodent-infecting Plasmodium proteins (see for details Table 2). Moreover, longer tandem repeat sequences then VGVAPG (2), such as VGVAPG (3) and VGVAPG (6) (respectively VGVAPGVGVAPG, VGVAPGVGVAPGVGVAPG and VGVAPGVGVAPGVGVAPGVGVAPGVGVAPGVGVAPG were abundantly found in primate elastins and primate-infecting Plasmodial proteins, allowing the conclusion that primate Plasmodium species specifically mimic the Chemotactic Domain of Elastin: VGVAPG

From the above findings, we concluded that the mouse exon 24 sequence (contrary to the primate exon 24 sequence with VGVAPG) did not specify a Chemotactic Domain of Elastin that is available in RODENT-infecting Plasmodial proteins.

The structural requirements for the ERC with the elastin-binding-protein EBP are well described in humans (see for example Uniprot entry P16278 isoform 2 and Hinek et al., Am J Hum Genet. 2000 July; 67 (1): 23-36; and also see Blanchevoye et al (2013) Interaction between the elastin peptide VGVAPG and human elastin binding protein, J Biol Chem 288:1317-28).

However, little is known about the structural requirements of the ERC with the elastin-binding-protein EBP in rodents. Here unpublished information is shared that the rodent EBP is considerably N-terminally truncated in the rodent EBP, as compared to the human EBP, with consequences for the preferred chemotactic domain of the rodent protein. In particular, the rodent EBP misses the first ~80 N-terminal amino acids from the human version, but still contains a typical VVGSPSAQDEASPL binding site for XGXXPG motif peptides. It is likely that said changes have some effect of XGXXPG-peptide binding to the rodent EBP when compared to the human EBP. Moreover, the human elastin gene has two fewer exons than mouse, attributable to the sequential loss of exons 34 and 35 during primate evolution (Szabó et al., (1999) Sequential loss of two neighboring exons of the tropoelastin gene during primate evolution. J. Mol. Evol. 49, 664-671), and in addition, although still contained in the human gene, exon 22 is never included in the human elastin transcript but is found in the mouse transcript.

When searching for the predicted GAVP motif that was found to cluster in the rodent Plasmodial proteins in the mouse and rat sequences, it was exon 22 (and not 34 or 35) that contained said GAVP motif in the preferred chemotactic domain of binding to the ERC (Table 2).

In particular GAVP was found in the PGAVPG motifs that were found repeatedly present in Plasmodial proteins derived from RODENT-infecting plasmodia, and also found repeatedly in elastins of rodents (see also tables 1 and 2), whereas crossover analyses showed no occurrences of a PGVAPG tandem repeat in primates nor of a VGAVPG tandem repat in rodents. Moreover, longer tandem repeat sequences AVPG₍₂₎, such as AVPG₍₃₎ and AVPG₍₆₎ (respectively AVPGAVPG, AVPGAVPGAVPG and AVPGAVPGAVPGAVPGAVPGAVPG were abundantly found in rodent elastins and rodent Plasmodial proteins, allowing the conclusion that rodent Plasmodium species preferred and specifically mimic another Chemotactic Domain of Elastin: PGAVPG

TABLE 2

| | Sequences | Uniprot protein entries | | | Note: |
|---|---|---|---|---|---|
| | searched | All | Elastin | Plasmodial | Accessed 5-7/07/2023 |
| human/maqaque | VGVAPG(2) | 339 | 170 | 51 | Only primate elastins and |
| EXON 24 derived | VGVAPG(3) | 307 | 167 | 41 | only primate-infecting |
| motifs | VGVAPG(6) | 113 | 45 | 10 | plasmodial proteins |
| | PGVGLAPG | 234 | 11 | 0 | Only human elastins |
| crossover | PGVAPG(2) | 72 | 33 | 0 | No rodent elastins |
| mouse/rat EXON | GVGGLPGG | 223 | 60 | 0 | Both primate as well as |
| 24 derived motifs | GVGGIPGG | 78 | 23 | 0 | rodent elastins and only |
| | | | | | primate-infecting plasmodial |
| | PGVGGVPG | 792 | 336 | 24 | proteins |
| | VGGVPG(2) | 166 | 155 | 24 | No rodent elastins |
| mouse/rat EXON | AVPG(2) | 588 | 133 | 18 | No primate elastins and only |
| 22 derived motifs | AVPG(3) | 109 | 18 | 14 | rodent-infecting plasmodial |
| | AVPG(6) | 24 | 6 | 7 | proteins |
| | ALPG(2) | 431 | 0 | 0 | |
| | ALPGAVPG | 167 | 11 | 0 | Only rodent elastins |
| | AVPGALPG | 282 | 12 | 0 | |
| | GVAPGGVAPG | 9 | 0 | 0 | |
| crossover | VGAVPG(2) | 4 | 3 | 0 | All bird elastins |

TABLE 3
Illustration of peptide search results with primate-infecting Plasmodial specific VGVAPG₍₃₎

| | | | VGVAPG |
|---|---|---|---|
| Uniprot entry | Protein name | Organism in which found | occurences |
| P15502 | ELASTIN | Homo sapiens (Human) | 6 |
| A0A1D5QE80 | ELASTIN | Macaca mulatta (Rhesus macaque) | 7 |
| A0A2I2YC63 | ELASTIN | Gorilla gorilla gorilla (Western lowland | 9 |
| | | gorilla) | |
| A0A2I3H867 | ELASTIN | Nomascus leucogenys (Northern | 7 |
| | | white-cheeked gibbon) | |
| | | (Hylobates leucogenys) | |
| A0A2I3RX20 | ELASTIN | Pan paniscus (Pygmy chimpanzee) | 8 |
| | | (Bonobo) | |
| A0A2J8Q8F5 | ELASTIN | Pan troglodytes (Chimpanzee) | 9 |
| A0A2J8XUS5 | ELASTIN | Pongo abelii (Sumatran orangutan) | 9 |
| | | (Pongo pygmaeus abelii) | |
| A0A2K5DV67 | ELASTIN | Aotus nancymaae (Ma's night monkey) | 3 |
| A0A2K5JW24 | ELASTIN | Colobus angolensis palliatus (Peters' | 6 |
| | | Angolan colobus) | |
| A0A2K5JWC6 | ELASTIN | Cercocebus atys (Sooty mangabey) | 7 |
| | | (Cercocebus torquatus atys) | |
| A0A2K5R675 | ELASTIN | Cebus imitator (Panamanian white- | 9 |
| | | faced capuchin) (Cebus capucinus | |
| | | imitator) | |
| A0A2K5XQM6 | ELASTIN | Mandrillus leucophaeus (Drill) | 7 |
| | | (Papio leucophaeus) | |
| A0A5F7ZFJ4 | ELASTIN | Macaca mulatta (Rhesus macaque) | 7 |
| A0A2K6BHI2 | ELASTIN | Macaca nemestrina (Pig-tailed | 7 |
| | | macaque) | |
| A0A2K6MP22 | ELASTIN | Rhinopithecus bieti (Black snub-nosed | 5 |
| | | monkey) (Pygathrix bieti) | |
| A0A2K6NWE2 | ELASTIN | Rhinopithecus roxellana (Golden | 7 |
| | | snub-nosed monkey) (Pygathrix roxellana) | |
| A0A2K6UXV2 | ELASTIN | Saimiri boliviensis boliviensis (Bolivian | 4 |
| | | squirrel monkey) | |
| A0A6J3FYD0 | ELASTIN | Sapajus apella (Brown-capped | 5 |
| | | capuchin) (Cebus apella) | |
| A0A7N9CKC2 | ELASTIN | Macaca fascicularis (Crab-eating | 7 |
| | | macaque) (Cynomolgus monkey) | |
| A0A480HHP7 | ELASTIN | Sus scrofa (Pig) | 4 |
| A0A5N4CXR5 | ELASTIN | Camelus dromedarius (Dromedary) | 3 |
| | | (Arabian camel) | |
| A0A6J3BAI6 | ELASTIN | Vicugna pacos (Alpaca) (Lama pacos) | 3 |
| A0A8B7K7S4 | ELASTIN | Camelus ferus (Wild bactrian camel) | 5 |
| | | (Camelus bactrianus ferus) | |
| A0A8C2RQD4 | ELASTIN | Capra hircus (Goat) | 3 |
| A0A8C3YJF1 | ELASTIN | Catagonus wagneri (Chacoan peccary) | 5 |
| A0A8C6FHG8 | ELASTIN | Moschus moschiferus (Siberian musk | 4 |
| | | deer) (Moschus sibiricus) | |
| A0A060RP03 | Erythrocyte membrane | Plasmodium reichenowi | 8 |
| | protein 1, EMP1 | | |
| A0A2P9C6H4 | Erythrocyte membrane | Plasmodium sp. gorilla clade G2 | 4 |
| | protein 1, EMP1 | | |
| A0A2P9GE15 | Erythrocyte membrane | Plasmodium sp. DRC-Itaito | 6 |
| | protein 1, EMP1 | | |
| A0A0E4FHZ7 | Serine-repeat antigen 5 (SERA) | Plasmodium vivax (malaria | 4 |
| | | parasite P. vivax) | |
| A0A0J9THX3 | Serine-repeat antigen 5 (SERA) | Plasmodium vivax Mauritania I | 3 |
| A0A1A8WTK7 | MSP7-like protein (MSRP2) | Plasmodium ovale curtisi | 5 |
| A0A1A8X7B3 | Serine-repeat antigen | Plasmodium malariae | 7 |
| A0A5P1A111 | Erythrocyte membrane | Plasmodium falciparum (malaria | 4 |
| | protein 1, EMP1 | parasite P. falciparum) | |
| W7K0V8 | Erythrocyte membrane | Plasmodium falciparum UGT5.1 | 8 |
| | protein 1, EMP1 | | |
| A0A1A8ZAZ8 | MSP7-like protein (MSRP2) | Plasmodium ovale wallikeri | 9 |
| A0A1B1DWS5 | KIR protein | Plasmodium coatneyi | 10 |
| A0A1B1DWU8 | Merozoite surface protein 1 | Plasmodium coatneyi | 5 |
| A0A1Y1JAI5 | Serine-repeat antigen 3 | Plasmodium gonderi | 15 |

TABLE 4
Illustration of peptide search results with rodent-infecting Plasmodial specific AVPG₍₃₎

| | | | AVPG |
|---|---|---|---|
| Uniprot entry | Protein name | Organism in which found | occurences |
| A0A8B7ERH7 | ELASTIN | Microcebus murinus (Gray mouse | 6 |
| | | lemur) (Lemur murinus) | |
| A0A6P5LS00 | ELASTIN | Phascolarctos cinereus (Koala) | 3 |
| A0A6P6ECR2 | ELASTIN | Octodon degus (Degu) (Sciurus degus) | 3 |
| A0A8C5KN57 | ELASTIN | Jaculus jaculus (Lesser Egyptian | 3 |
| | | jerboa) | |
| A0A286XYG7 | ELASTIN | Cavia porcellus (Guinea pig) | 3 |
| A0A673UG76 | ELASTIN | Suricata suricatta (Meerkat) | 4 |
| P13828 | Merozoite surface protein 1 | Plasmodium yoelii yoelii | 6 |
| V7PP57 | Merozoite surface protein 1 | Plasmodium yoelii 17X | 4 |
| A0A081ICC6 | CCAT-binding transcription | Plasmodium vinckei vinckei | 3 |
| | factor-like protein | | |
| A0A6V7SK24 | CCAT-binding transcription | Plasmodium vinckei vinckei | 5 |
| | factor-like protein | | |
| A0A6V7RXG9 | CCAT-binding transcription | Plasmodium vinckei lentum | 7 |
| | factor-like protein | | |
| W7AIJ5 | CCAT-binding transcription | Plasmodium vinckei petteri | 4 |
| | factor-like protein | | |
| A0A1C6X986 | CCAT-binding transcription | Plasmodium chabaudi chabaudi | 7 |
| | factor-like protein | | |
| A0A1D3LBQ3 | Asparagine-rich antigen | Plasmodium chabaudi chabaudi | 3 |
| A0A1C6XH03 | CCAT-binding transcription | Plasmodium chabaudi adami | 8 |
| | factor-like protein | | |

### Example 2

Peptide Synthesis and suitable peptides with the required chemotactic domain.

In describing protein or peptide composition, structure, and function herein, reference is made to amino acids. In the present specification, amino acid residues are expressed by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; lie: isoleucine residue; Lys: lysine residue; Leu: leucine residue; Met: methionine residue; Asn: asparagine residue; Pro: proline residue; Gin: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

Modulator peptides for treatment of Insulin secretion-related disorders (ISRDs) as provided herein preferably have a motif selected from the group at least functionally equivalent to any of a peptide with motif VGVAPG, vGVAPG, vGvAPG, vGVaPG, vGvaPG, LGGGPG, IGGGPG, PGAYPG, pGAYPG, pGaYPG, pGAyPG or pGayPG, allowing targeting to the elastin receptor on the surface of cells. Typical example are peptides such as RREAEDLQVGQVELGGGPGAGSLQPLALEGSLQKR, PGAIPG, GAVPG, GVLPG, GGVPG, GVVPG, VGVAPG, VVGPGA, VGVAPG, LGGGPG, QGQLPG, PGAYPG, QGVLPA, AQGVAPG, LQGVAPG, AQGVLPG, LQGVLPG, AQGVAPGQ, LOGVAPGQ, AQGVLPGQ, LQGVLPGQ, VGVAPGVGVAPG, LGGGPGAGSLQP and PGAYPGAPAPGV, VGVAPGVGVAPGVGVAPGVGVAPG, QVGQVELGGGPG and GAYPGAPGAYPG, VGVAPGVGVAPGVGVAPG, QVGQVELGGGPGAGSLQP and GAYPGAPGAYPGAPAPGV, VGVAPG, GVAPGV, VAPGVG, APGVGV, PGVGVA, GVGVAP, PGAIPG, LGTIPG, LGGGPGAG, GGGPGAG, GGGP, GGGPG, GAGPG, GGGPE, GAIPG, GGVPG, GVAPG, YTTGKLPYGYGPGG, YGARPGVGVGIP, PGFGAVPGA, GVYPG, GFGPG, GVLPG, GAIPG, PGAIPG, PGAVPG, VGAMPG, VGSLPG, VGMAPG, VPGVG, IPGVG, VGSLPG, VGVAPG, VGVPG, AGAIPG, VPGV, LGITPG, GDNP, GAIP, GKVP, GVQP, GVGP, GFGP, GGIP, GVAP, GIGP, GAGP, GGIPP, GQFP, GLSP, GGPQP, GPQPG, GGPQPG, GIPP, GGIPP, GIPPA, GGIPPA, EAEDLQVGQVELGGGPGAGSLQPL, DLQVGQVELGGGPGAGSLQP, LVGQVELGGGPGAGSLQPL, QVGQVELGGGPGAGSLQPL, ELGGGPGAGSLQPL, DLQVGQVELGGGPGAGSLQPLALEGSLQ, GQVELGGGPGAGSLQPLALEGSLQ, LGGGPGAGSLQPLALEGSLO, LVGQVELGGGPGAGSLQPL, LGGGPGAGSLQPL, and LQVGQVELGGGPG, LQVGQVELGG and/or GGPGAGSLQPL, PQGWPGAWGNQPAGAGGYPGASYPGAYPGQAPPGAYPGQAPPGAYPGAPGAYPGAPAPGVYPGPPSGPGAYPSS GQPSATGAYPATGPYGAPAGPLIVPYNLPLPGGVVPRM, AGGYPGASYPGAYPGQAPPGAYPGQAPPGAYP, MMRVLQAVLPPLPQVVCTYR, GAPGAYPGAPAPGVYPGPPSGPGAYPSSGQPSATGAYPATGPY and GAPAGPLIVPYNLPLPGGVVP, GAYPGAPGAYPGAPAPGV and PGAYPG, PGAYPGQAPPGAYPG, PGAYPGQA and GQAPPGAYPG, vGVAPGVGVAPGVGVApG, vGVAPGVGVAPGEGSLQa, qVGQVELGGGPGAGSLQp, qVGQVELGGGPGEGSLQa, GaYPGAPGAYPGEGSLQav, GaYPGAPGAYPGAPAPGv, vGvAPGVGVAPGVGVapG, qvGQVELGGGPGAGSLqp, GayPGAPGAYPGAPApGv vGvaPGVGVAPGVGvapG, qvGqVELGGGPGAGSIqp and GaypGAPGAYPGAPapgV, gpavgvgpavgvgpavgv, pqlsgagpgggIevqgvq, vgpapagpyagpagpyay, aqlsgegpavgvgpavgv, aqlsgegpgggIevqgvq, aqlsgegpyagpagpyay, vGVAPGVGVApG, vGVAPGEGSLQa, qGGGPGAGSLQp, qGGGPGEGSLQa, GaAYPGEGSLQav, GaYPGAAPAPGv, vGvAPGVGVapG, qvGGGPGAGSLqp, GayPGAAPApGv vGvaPGVGvapG, qvGqVELGGGPGqp and GaypGAAPapgV, gpavgvgpavgv, pqlsgegpgggIvq, vgpapgpagpyay, aqlsgegpavgv, aqlsgegpggqgvq, aqlsgegpagpyay or a functional equivalent thereof.

Note that, except for glycine, all standard α-amino acids may exist in either of two optical isomers, which are the mirror image of one other; these are called L- and D-amino acids. Herein, when defining an amino acid sequence of a peptide in the one letter code, lowercase letters indicate D-amino acids, whereas uppercase letters indicate L-amino acids, with the notable exception of glycine, wherein both g and/or G indicate the amino acid glycine of which no optical isomers, and thus no difference between D- or L-forms, exist.

GxxP-motif peptides comprising PG-domain ERC-binding motifs such as VGVAPG, GVAPGV, VAPGVG, APGVGV, PGVGVA, GVGVAP, PGAIPG, LGTIPG, LGGGPGAG, GGGPGAG, GGGP, GGGPG, GAGPG, GGGPE, GAIPG, GGVPG, GVAPG, YTTGKLPYGYGPGG, YGARPGVGVGIP, PGFGAVPGA, GVYPG, GFGPG, GVLPG, GAIPG, PGAIPG, PGAVPG, VGAMPG, VGSLPG, VGMAPG, VPGVG, IPGVG, VGSLPG, VGVAPG, VGVPG, AGAIPG, VPGV, LGITPG, GDNP, GAIP, GKVP, GVQP, GVGP, GFGP, GGIP, GVAP, GIGP, GAGP, GGIPP, GQFP, GLSP, GGPQP, GPQPG, GGPQPG, GIPP, GGIPP, GIPPA, GGIPPA, AQGVAPG, LQGVAPG, AQGVLPG, LQGVLPG, AQGVAPGQ, LOGVAPGQ, AQGVLPGQ, LQGVLPGQ, or retro- or inverso- or retro-inverso- or mixed L-/D-chemotactic domain variants thereof are synthesized according to classical solid phase synthesis or by alternative methods known in the art. V14 peptide, a peptide reproducing the sequence of S-Gal interacting with elastin peptides bearing the motif GxxP, is obtained from Neosystem (Strasbourg, France). Alternatively, V14 peptide and variants thereof are synthesized as described herein. Purity of the peptides is confirmed by high performance liquid chromatography and by fast atom bombardment mass spectrometry.

Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures. Functional distinctions may also be made between peptides and proteins. Peptides, however, may be subdivided into peptides, which have few amino acids (e.g., 2 to 30-50), and polypeptides, which have many amino acids (>50). Proteins are formed from one or more polypeptides joined together. Hence, proteins essentially are very large peptides. In fact, most researchers, as well as this application, use the term peptide to refer specifically to peptides, or otherwise relatively short amino acid chains (<51 amino acids), with the term polypeptide being used to describe proteins, or chains of >50 or much more amino acids.

### Example 3

### The Elastin Receptor Complex (ERC, Herein Also Identified as Elastin Receptor)

The elastin receptor is involved in chemotaxis of leukocytes and activation of matrix-metallo-proteinases, in endothelial cell migration and angiogenesis and in proliferation of fibroblasts and vascular smooth-muscle cells. The receptor is activated by (proteolytic) fragments of extracellular matrix in granulating tissue after tissue injury or inflammation, fulfilling handyman jobs towards tissue repair.

The receptor consists of an alternatively spliced variant of beta-galactosidase. It is well known to bid to any peptide having a hexapeptide X-Gly-X-X-Pro-Gly (XGXXPG) motif in (proteolytic fragments of) extracellular matrix proteins such as elastin and fibrillin-1 4. The best-known representative of the motif is hexapeptide VGVAPG found in (tropo) elastin, but many other biologically active peptides conforming to the signature sequence XGXXPG, generally called elastin peptides, have been reported as agonist. WO2018141970 identifies various other peptides and proteins, such as C-peptide, notably its midportion, and galectin-3, notably its N-terminal fragment, as ligands of the ERC. A minimally essential sequence for biological activity is GXXP, with the peptide at P adopting a type Vill beta-turn, a condition considered always met when the amino acid following the proline (at its N-terminal side) is glycine. Lactose and/or V14 peptide VVGSPSAQDEASPL corresponding to the peptide binding site of the receptor, is used to antagonize elastin peptide binding.

The elastin receptor consists of a complex of the elastin binding protein (EBP) with neuraminidase (Neu-1) and protective protein-cathepsin A (PPCA) on the cell surface. After binding to its ligand, the complex internalizes to endosomal compartments in the cell and triggers numerous cellular responses. In mice, exogenous elastin peptides with motif GXXP potentiate atherosclerosis through Neu-1 and regulate insulin resistance due to an interaction between Neu-1 and the insulin receptor. Moreover, in mice, PPCA is required for assembly of elastic fibers and inactivation of endothelin-1, impaired activation of endothelin-1 resulting in hypertension.

Proof of concept study performed at Aquilo B V for Resiliun B V to evaluate the efficacy of elastin fragment and related GXXP peptide fragments from C-peptide and Galectin-3 on Neu-1 mediated angiogenesis and insulin receptor sensitivity. Active desialylated NEU1 inhibits angiogenesis (J Biol Chem. 2014 Mar. 28;289 (13): 9121-35) and this activity is blocked by treatment with DANA.

Sialidases, also known as neuraminidases (NEUs), are a family of enzymes responsible for the regulation of sialic acid expression on the cell surface by removing sialic acid from endogenous glycoconjugates (Xiao et al., Proc Natl Acad Sci USA. 2016 Sep. 13; 113 (37): 10304-9.). In addition, terminal sialic acid promotes the integrity of the endothelial barrier (Cioffi et al., Am J Physiol Lung Cell Mol Physiol. 2012 May 15; 302 (10): L1067-77). The most famous enzyme in this family is influenza neuraminidase, which was first discovered in the 1950s. Neuraminidases are a large family found in many organisms, including viruses, bacteria, fungi, protozoa, birds, and mammals (Pshezhetsky and Ashmarina, Biochemistry (Mosc). 2013 July; 78 (7): 736-45.). Neuraminidase is found in many mammalian organs. NEU1 is highest expressed in the kidneys, pancreas, skeletal muscle, liver, lungs, placenta, and brain; NEU2 is mainly found in muscle tissue; NEU3 is highest expressed in adrenal glands, skeletal muscle, heart, testes, and thymus; NEU4 is highest expressed in the brain, skeletal muscle, heart, placenta, and liver (Pshezhetsky and Ashmarina, ibid). In cells, NEU1 is localized in lysosomes and on plasma membranes to participate in exocytosis, immune response, phagocytosis, and elastic fiber assembly. As neuraminidase 1 (NEU1) cleaves terminal sialic acids of glycoconjugates it also modulates the structure and activity of cellular surface receptors affecting many and diverse pathways, among which:

Dridi et al (Positive Regulation of Insulin Signaling by Neuraminidase 1, Diabetes 2013 July; 62 (7): 2338-2346.) demonstrated that NEU1 activates the insulin receptor. Insulin signaling is a key event in the regulation of glucose homeostasis, the signaling cascade starts from binding of insulin to the cell surface insulin receptor kinase (IRK). The receptor is rapidly activated, autophosphorylated at specific tyrosine residues, and internalized into endosomes. The activated IRK phosphorylates substrates, including IRS-1 to-4, which bind to effector molecules such as phosphatidylinositol 3-kinase (PI3K), resulting in their activation (reviewed in Taniguchi et al., Critical nodes in signaling pathways: insights into insulin action. Nat Rev Mol Cell Biol 2006; 7:85-96). Dridi et al show that Neu-1 desialylates and activates the insulin receptor kinase (IRK) through desialylation of the glycan chains attached to the β-chain of human IRK, thus providing a feedback mechanism for the regulation of glucose uptake. Pretreatment with the Neu-1 inhibitor 2,3-dehydro-2-deoxy-N-acetylneuraminic acid (DANA) significantly reduced desialylation of the IRK, confirming that Neu1 indeed removes sialic acid residues from the glycan chains of the B-subunit of the receptor.

Angiogenesis is a tightly orchestrated process in which proangiogenic and antiangiogenic factors are released to bind to their cognate endothelial cell (EC) surface receptors such as the elastin receptor complex. The proangiogenic receptor-ligand interactions are coupled to EC disengagement from neighboring ECs and protease-mediated degradation of the underlying EC extracellular matrix (ECM) and release of elastin-derived peptides from the disrupted ECM. These signaling and proteolytic events permit ECs to migrate through the ECM toward the proangiogenic stimulus generated from the elastin-derived peptides, notably those bearing a XGXXPG motif. Proliferative signals expand the EC population, which, coupled with ECM remodeling, lead to EC-EC re-engagement with capillary-like tube formation. A number of these proangiogenic processes involve altered glycosylation patterns and specific carbohydrate-mediated recognition events.

The surface of endothelial cells is highly sialylated, and changes in the state of sialylation, affecting angiogenesis. Human NEU1 is the most abundant sialidase, found in the matrix gel system to inhibit angiogenesis (Lee et al., Biol Chem. 2014 Mar. 28; 289 (13): 9121-35.). It is also the first sialidase to be described as an angiogenesis regulator (Glanz et al., Eur J Pharmacol. 2019 Jan. 5; 842 ( ) 345-350.). The highly sialylated vascular endothelial surface undergoes changes in sialylation upon adopting the migratory/angiogenic phenotype. Cross et al., established endothelial cell (EC) cell surface expression of NEU1 sialidase (2012; EU1 and NEU3 sialidase activity expressed in human lung microvascular endothelia. NEU1 restrains endothelial cell migration whereas NEU3 does not. J. Biol. Chem. 287, 15966-15980). It was further found that NEU1 regulates EC capillary-like tube formation on a Matrigel substrate in human pulmonary microvascular ECs (HPMECs). Prior silencing of NEU1 did not alter tube formation, indicating that in a resting state Neu-1 is not involved.

Elastin-induced atherosclerosis depends on sialidase activity and the EBP-Cathepsin A-Neu-1 elastin-receptor complex (ERC). Therefore, elastin can be used as an enhancer of atherosclerosis (Glanz et al., Eur J Pharmacol. 2019 Jan. 5; 842 ( ) 345-350.). The elastin-receptor complex is also essential for the ability of fibroblasts to respond to elastin degradation. The combination of elastin-derived peptides and the elastin-receptor complex activates the intracellular signaling cascade, including the activation of extracellular regulated protein kinases (ERK) 1/2 and the production of pro-MMP-1 (Parker and Kohler, ACS Chem Biol. 2010 Jan. 15; 5 (1): 35-46.). Sialic acid content in the endothelial glycocalyx plays an important role in the development of atherosclerosis, and the regulation of leukocyte and platelet adhesion, mechanical transduction, and endothelial cell absorption of low-density lipoprotein (Frontiers of Pharmacology 2020; 11:590614).

### Example 4

As measuring insulin resistance in vitro is a cumbersome feat, the effect of three elastin fragment peptides and related GXXP-peptides on endothelial cell sprouting were assessed instead. Endothelial cells (Hpmec, n=4) were serum starved for 24 hours, trypsinised and seeded in growth factor-reduced Matrigel. Cells were treated with vehicle or increasing concentration of peptide and digital images have been acquired after 4 h incubation with a second assessment 24 h after incubation. Sprouting by means of mean total tube length and mean total branch length per field of view were quantified with ImageJ software.

### Methods

### Cell Culture

Human pulmonary microvascular endothelial cells (Hpmec) were cultered in EBM-2 medium (Lonza, CC-3156) supplemented with 10% FBS and all components present in the bullet kit (Lonza, CC-4147) containing human recombinant FGF-B, human recombinant VEGF, human recombinant R3-IGF-1, ascorbic acid, human recombinant EGF and GA-1000 (Gentamicin sulfate-Amphotericin). 24 hours prior to the assay Hpmec were starved in EBM-2 medium containing 0.5% FBS instead of 10% FBS. For introduction in the angiogenesis assay, endothelial cells were washed 2 times with warm PBS, trypsinized and resuspended in serum free EBM-2 medium.

### Angiogenesis Assay

Matrigel (Fisher Scientific, Landsmeer, The Netherlands #11523550) was diluted 1:1 in serum-free EBM-2 medium and plated in a 96-well plate (50 μL). After polymerization, the cells were combined with the experimental compounds and added on top of the Matrigel (100 μL). Pictures of the microvascular network were taken after 4 and 24 hours of incubation with a 4x objective.

### Analysis

Pictures were analyzed using the plug-in angiogenesis analyzer in ImageJ. For the analyses, total tube length and total branch length Peptides 3, 6 and 8 are tested; these are 18-meric GXXP-peptide fragments (VGVAPGVGVAPGVGVAPG, QVGQVELGGGPGAGSLQP and GAYPGAPGAYPGAPAPGV) taken from the exon 24 region of human elastin, from the mid-portion of human C-peptide, and from the N-terminal portion of Galectin-3, respectively.

### Results and Discussion

See FIG. 4. Microvascular angiogenesis assay of human pulmonary microvascular endothelial cells grown in Matrigel and in response to increasing concentrations of elastin fragment peptides 3, 6 and 8, (FIGS. 4a, 4b and 4c, respectively). Data shown are total tube length measurements and total branch length measurements at t=24 h. * p<0.05; ** p<0.01; *** p<0.001. At 4 h no distinct results were obtained. Also, as expected, DANA significantly potentiated the angiogenic effects of peptide 3, at t=16, 24 and 40h (P=0.48, 0.011 and 0.032, respectively).

Of interest is that for these biologically active peptides a biphasic concentration response curve was apparent, with maximum effect usually obtained around 4,000 to 40,000 nM and the higher concentration of 400,000 nM being less effective. The statistical analyses support this conclusion. Three possible explanations come to mind with such a biphasic nature being a) desensitization/internalization of the target receptor at higher concentrations leading to loss of effect; b) the presence of a second receptor with lower affinity and functionally opposite effects; c) toxicity of the peptide at the highest concentrations. Explanation A appears the most likely one, as the biphasic nature was strongest for the peptides with the strongest functional effects. Toxicity effects would be expected to be more ubiquitously present, whereas a functional effect of a second receptor is less likely in view of the structurally quite diverse nature of the peptides, making such a common off-target effect less likely. Na⁺, K⁺-ATPase is an ubiquitous membrane enzyme that allows the extrusion of three sodium ions from the cell and two potassium ions from the extracellular fluid. Na⁺,K(+)-ATPase activity is decreased in the red blood cell membranes of type 1 diabetic individuals, irrespective of the degree of diabetic control. It is less impaired or even normal in those of type 2 diabetic patients. Vague et al., (Na+,K(+)-ATPase, and diabetes. Exp Diabesity Res. 2004 January-Mar; 5 (1): 37-50. doi: 10.1080/15438600490424514. PMID: 15198370; PMCID: PMC2478626.) have shown that in the red blood cells of type 2 diabetic patients, Na⁺, K(+)-ATPase activity was strongly related to blood C-peptide levels in non-insulin-treated patients (in whom C-peptide concentration reflects that of insulin) as well as in insulin-treated patients. Short-term C-peptide infusion to type 1 diabetic patients restores normal Na+,K(+)-ATPase activity. Islet transplantation, which restores endogenous C-peptide secretion, enhances Na+, K(+)-ATPase activity proportionally to the rise in C-peptide. This C-peptide effect is not indirect. In fact, incubation of diabetic red blood cells with C-peptide at physiological concentration leads to an increase of Na⁺, K(+)-ATPase activity. In isolated proximal tubules of rats or in the medullary thick ascending limb of the kidney, C-peptide stimulates in a dose-dependent manner Na+,K(+)-ATPase activity.

### Example 5

PG-domain C-peptide, a cleavage product of the proinsulin molecule, has long been regarded as biologically inert, serving merely as a surrogate marker for insulin release. Recent findings demonstrate both a physiological and protective role of C-peptide when administered to individuals with type I diabetes. Data indicates that C-peptide appears to bind in nanomolar concentrations to a cell surface receptor which is most likely to be G-protein coupled. Binding of C-peptide initiates multiple cellular effects, evoking a rise in intracellular calcium, increased PI-3-kinase activity, stimulation of the Na⁺/K+ ATPase, increased eNOS transcription, and activation of the MAPK signaling pathway. These cell-signaling effects have been studied in multiple cell types from multiple tissues. Overall, these observations raise the possibility that C-peptide may serve as a potential therapeutic agent for the treatment or prevention of long-term complications associated with diabetes. Clearly if PG-domain C-peptide has peptide hormone-like actions, it is necessary to postulate the existence of a receptor. Indeed, specific, and displaceable binding of 1251-labelled C-peptide was first demonstrated by Flatt et al. (Biosci Rep. 1986 February; 6 (2): 193-9.) who derived a curvilinear Scatchard plot of specific C-peptide binding to pancreatic islet B-cells.

Subsequently, using retro sequence and all-D-amino acid C-peptide enantiomers, Ido et al. (Science. 1997 Jul. 25; 277 (5325): 563-6.) suggested that C-peptide-induced improvements in nerve function and vascular permeability blood flow in diabetes through increases in Na⁺, K⁺-ATPase activity did not result from C-peptide binding in a stereospecific manner to a receptor. Instead, it was hypothesized that biological activity of C-peptide was dependent on poorly defined membrane interactions that took place due to structural features related to the C-peptide sequence, but independent of its direction or chirality. A mid-portion sequence of C-peptide, largely conserved, and comprising a high proportion of nonpolar amino acids flanking a C16 proline was implicated in this activity.

Other investigators have also examined C-peptide fragment bioactivity using Na⁺, K⁺-ATPase activity in rat renal tubular segments as a readout (Diabetologia. 1998 March; 41 (3): 287-91.) Two segments of C-peptide demonstrated functional importance. The rat C-peptide carboxy terminal pentapeptide, EVARQ, elicited 100% of the activity of intact C-peptide whereas the remaining portion of the molecule, lacking said 5 amino acid terminal sequence, was totally inactive. In this rat system, the terminal pentapeptide of human C-peptide (EGSLQ) elicited 75% activity; and notably the glutamic acid at position 1 and the glutamine at position 5 are generally conserved in mammals. Similar well-defined C-terminal functional sequences are also found in gastrin and cholecystokinin. Overall, the behavior of the C-terminal pentapeptide in these studies was typical of a peptide ligand interacting with a specific receptor. In contrast, several C-peptide mid-region sequences were able to partially recapitulate the activity of the intact molecule but exhibited rather different properties. Activity relating to this region was not seen with the des-(27-31)-C-peptide, and several non-natural D-amino acids containing sequences showed some activity. The activity of these segments decreased if they were greater than 9 amino acids in length. This behavior was earlier thought not reminiscent of peptide-receptor interactions, but similar to the nonspecific type interactions of C-peptide with plasma membranes postulated by Ido et al. (ibid).

How the balance of activity provided by these two domain regions of the C-peptide molecule is manifest in vivo is currently not well understood. Very recent work suggests that efficient activation of signaling pathways requires the presence of conserved glutamic acid residues at positions 3, 11, and 27 of C-peptide, and the presence of helix-promoting residues in the N-terminal segment (Henriksson et al., Cell Mol Life Sci. 2005 August; 62 (15): 1772-8). Taken together with here not reviewed data relating to its N-terminus, the overall picture now emerging of the structure-activity relationship of the C-peptide molecule is one of a tripartite structure where the terminal sections are involved in functional interactions, with the PG-domain mid-region forming an ERC-binding segment.

The invention herewith provides use of a distinct and newly emerging class of drugs: peptide modulators of mTOR that act as autophagy inhibiting compounds for use in inducing the activities required to establish the glycemic control to combat hyperinsulinism, that even occur in those patients that are otherwise under proper glycemic control. When autophagy is inhibited and therewith mTOR is activated following amino acid consumption by fat or muscle cells derived from these peptide modulators provided herein the sustained simulation of downstream S6K₁ increases IRS1 Ser307 phosphorylation. This decreases its activity and responsiveness to insulin, thereby rendering the fat or muscle cell insulin resistant. Similarly, when endothelial cells such as beta-cells of vascular endothelial cells are treated with these peptide modulators of mTOR as provided herein the sustained simulation of downstream S6K1 increases IRS1 Ser307 phosphorylation induces cell proliferation. This proliferation may be measured experimentally by for example detecting Ki-67 activity in those cells. Provided is an autophagy inhibiting peptide modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). To target such peptide modulators to cells, such peptides may be provided with an xGxxPG motif as discussed above well. In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and valine (V). In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and leucine (L). In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), leucine (L), and proline (P). In a preferred embodiment, provided is an autophagy inhibiting modulator of mTOR for use in inducing increased glycaemia in prevention or treatment of a hyperinsulinism of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), valine (V), leucine (L), and proline (P). It is in particular preferred that such an autophagy-inhibiting peptide modulator of mTOR according to the invention is used when said subject is also treated to achieve or maintain glycemic control, especially when said subject is also treated with an insulin to achieve or maintain glycemic control. Provided is a method for identifying a source of, preferably L-proteinogenic, amino acids, preferably a peptide, capable of inducing increased glycaemia in prevention or treatment of a hyperinsulinism of a subject, comprising providing cells with a peptide comprising L-proteinogenic amino acids, said amino acids for least 50% selected from the group of alanine (in one letter code: A), glutamine (Q), glycine (G), valine (V), leucine (L), isoleucine (I), proline (P), arginine (R) and asparagine (N), and determining angiogenic activity in a sprouting assay. Angiogenic activity can be determined by assessing capillary-tube formation as well as by assessing capillary branch formation, as provided herein. It is preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50%, more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R) and asparagine (N).). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N), more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). The invention also provides a method for inducing increased glycaemia in prevention or treatment of a hyperinsulinism of a subject, comprising providing cells with a peptide as a source of, preferably L-proteinogenic, amino acids, said peptide consisting of amino acids that are for at least 50%, more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). The invention also provides a method of treatment according to the invention, wherein said source of, preferably L-proteinogenic, amino acids, preferably a peptide, is identifiable with a method as provided herein. The invention in particular provides a method of treatment according to the invention wherein said source or peptide has an amino acid sequence that is derived from a peptide or protein shown or suspected of having activities. Often, in the proteome of an organism, such angiogenic sequences in proteins are located close to or between more N- and C-terminal located arginine (R) or lysine (K) residues, allowing enzymes such as convertases to cut out said amino acid sequence capable of inducing increased glycaemia in prevention or treatment of a hyperinsulinism of a subject so that it can be used as an autophagy inhibiting peptide modulator of mTOR. It is preferred to correlate peptide use with the species of cells it is used in. For example, it is preferred that if the peptides are used in human cells as an autophagy inhibiting peptide modulator of mTOR, said peptides derive from the human proteome. Likewise, in cells of another species, such as species X, it is preferred to use peptide that derives from the species X proteome. In a preferred embodiment, provided is said autophagy-inhibiting peptide which has an amino acid sequence that is derived from a chorionic gonadotropin (CG), a protein involved in activities in pregnancies, preferably said peptide is derived from a beta-chain of CG, preferably from a loop 2 of said beta-chain. Human CG (hCG) has an amino acid sequence capable of inducing increased glycaemia in prevention or treatment of a hyperinsulinism of a subject MTRVLOGVLPALPQVVCNYR wherein the core sequence is located between N- and C-terminal located arginine (R) residues. Furthermore, this core comprises an ERC-binding motif xGxxPx. Splitting up this motif facilitates use of hCG derivatives LQGV, VLPALP AQGV, LAGV, LOAV, LOGA, ALPALP, VAPALP, VLAALP, VLPAAP, and VLPALA as autophagy inhibiting peptide modulators in combination with rapid- and short-acting insulin preparations, in particular to exert both glycemic induction as well as glycemic control. Peptide VLQGVLPALPQVV, for example, finds a better use in combination with intermediate- and long-acting insulin preparations where it is released more slowly and more in line with the release rates of the insulins. In another preferred embodiment, provided is said autophagy-inhibiting peptide wherein said peptide has an amino acid sequence capable of inducing increased glycaemia in prevention or treatment of a hyperinsulinism of a subject that is derived from a C-peptide. In the pre-pro-insulin molecule human C-peptide has sequence RREAEDLQVGQVELGGGPGAGSLQPLALEGSLQKR, indeed the core sequence is flanked by arginine (R) or lysine (K) residues. Furthermore, this core comprises an ERC-binding motif xGxxPG. As ERC-binding motifs are involved in coacervation trough oligomerization of peptides having these sequences, such autophagy inhibiting peptides having an ERG-binding motif typically have a slower release rate when injected and are typically more useful and preferred under circumstances wherein such slow-release is desired, such as in combination formulations with intermediate- or, more preferably, long-acting insulins. Coacervation typically involves aggregation of colloidal droplets of the peptides held together by electrostatic attractive forces, which explains the slow-release nature of such ERC-motif comprising peptides when injected, at least in comparison with peptides without ERC-motif and not showing coacervation, that typically have faster release characteristics. Preferred C-peptide fragment peptides for use as autophagy inhibiting peptide modulator of mTOR in treatment of angiogenic dysfunction, in particular in circumstances of C-peptide deficiency are preferably isolated and/or synthetic, preferably non-PEGylated, C-peptide fragments EAEDLQVGQVELGGGPGAGSLQPL, DLQVGQVELGGGPGAGSLQP, LVGQVELGGGPGAGSLQPL, QVGQVELGGGPGAGSLQPL, ELGGGPGAGSLQPL, DLQVGQVELGGGPGAGSLQPLALEGSLQ, GQVELGGGPGAGSLQPLALEGSLQ, and LGGGPGAGSLQPLALEGSLQ,, LVGQVELGGGPGAGSLQPL, LGGGPGAGSLQPL, and LQVGQVELGGGPG, and functional equivalents all comprising an xGxxPG motif are thus most suitable and preferred for inclusion in or combination with an intermediate- or long-acting insulin. LQVGQVELGG and/or GGPGAGSLQPL and functional equivalents not comprising an xGxxPG motif are thus most suitable and preferred for inclusion in or combination with a rapid- or short-acting insulin. In another preferred embodiment, provided is said autophagy-inhibiting peptide wherein said peptide has an amino acid sequence capable of inducing increased glycaemia in prevention or treatment of a hyperinsulinism of a subject that is derived from a galectin-3. The mature N-terminal fragment of human galectin-3 PQGWPGAWGNQPAGAGGYPGASYPGAYPGQAPPGAYPGQAPPGAYPGAPGAYPGAPAPGVYPGPPSGPGAYPSS GQPSATGAYPATGPYGAPAGPLIVPYNLPLPGGVVPRM, is typically characterized by multiple ERC-binding motifs xGxxPG, core sequences are also liberated by hydrolyses. Typically (Pineda et al., Trypanosoma cruzi cleaves galectin-3 N-terminal domain to suppress its innate microbicidal activity. Clin Exp Immunol. 2020 February; 199 (2): 216-229.), human pathogen T. cruzi not only binds, but also hydrolyses human N-terminal galectin-3. Remarkably, this mechanism prevents galectin-3-mediated parasite death, suggesting that T. cruzi may have developed complex strategies to modulate galectin-3 functions to successfully infect, survive and thrive within its mammalian hosts. In fact, non-pathogenic T. rangeli binds galectin-3 but does not modify protein structure. Thus T. cruzi cleaves the N-terminal collagen-like domain, rendering the C-terminal galectin-3 unable to oligomerize via coacervation, and allowing N-terminal fragments with motif xGxxPG to interact with the ERC and modulate mTOR to the good of the parasite. Specifically, the N-terminal sequences of 3 fragments obtained through parasite hydrolyses were: band 1: AGGYPGASYPG, band 2: GAPGAYPGAP and band 3: GAPAGPLIVP, indicating mTOR modulating characteristics of galectin-3 N-terminal peptide fragments derived from AGGYPGASYPGAYPGQAPPGAYPGQAPPGAYP, GAPGAYPGAPAPGVYPGPPSGPGAYPSSGQPSATGAYPATGPY and GAPAGPLIVPYNLPLPGGVVP in man. For example, fragments GAYPGAPGAYPGAPAPGV and PGAYPG have core activities, as demonstrated herein, making PGAYPGQAPPGAYPG, PGAYPGQA and GQAPPGAYPG also useful autophagy inhibiting peptides for use in man, in particular when included in or with intermediate- or long-acting insulins. The invention also provides a method of treatment and use therein according to the invention wherein said peptide has an amino acid sequence that is derived from a lutropin (LH), a protein often monthly involved in activities in women, preferably said peptide is derived from a beta-chain of LH, preferably from a loop 2 of said beta-chain. Beta-2-loop of human LH (hLH) has an amino acid sequence capable of inducing increased glycaemia in prevention or treatment of a hyperinsulinism of a subject MMRVLQAVLPPLPQVVCTYR wherein the core sequence is located between N- and C-terminal located arginine (R) residues. Furthermore, this core does not comprise an ERC-binding motif xGxxPx, facilitating use of LH-derived sequences, such as VLQAVLPPLPQVV, VLQAVLP, VLQA, LQAVLP, LQAV, PLPQVV, and PPLQV, and further derivatives as mTOR modulator in rapid- and short-acting insulin formulations. The invention also provides a method of treatment and use therein according to the invention wherein said peptide has an amino acid sequence that is derived from an extra-cellular matrix protein (ECM). ECM proteins comprise an abundant source of autophagy inhibiting amino acids and are useful as templates for design of autophagy-inhibiting peptides. Proline as substrate is stored in elastin and collagen in extracellular matrix, connective tissue, and bone and it is rapidly released from this reservoir by the sequential action of matrix metalloproteinases, peptidases, elastases and prolidase. As the only proteinogenic secondary amino acid, proline has special biological effects, serves as a regulator of all protein-protein interactions and responses to metabolic stress, and initiates a variety of downstream metabolic activities, including autophagy (Kadowaki et al. Nutrient control of macroautophagy in mammalian cells. Mol Aspects Med. 2006 October-Dec; 27 (5-6): 426-43). The invention also provides a method of treatment and use therein according to the invention wherein said extra-cellular matrix protein is an elastin. Typical core-activities have been demonstrated with peptides VGVAPG and VGVAPGVGVAPGVGVAPG herein, both derived from the exon 24 region of human elastin. The invention also provides a method of treatment and use therein according to the invention wherein said extra-cellular matrix protein is a collagen, Collagen is rife with PGP sequences that may be useful. Typical for some collagens (here examples from human COL6A5 are shown) that comprise core peptides flanked by R or K, such as RRAQGVPQIAVLVTHR, identifying core sequence AQGVPQIAVLV, and derivatives such as AQGVPQ, AQGVPQI, AQGVPQIA,GVPQIAVLV, PQIAVLV, IAVLV, and others. More of such sequences may be found in collagen stretches such as (in human COL6A5) RGAPGQYGEKGFPGDPGNPGQNNNIKGQKGSKGEQGRQGRSGQKGVQGSPSSRGSRGREGQRGLRGVSGEPGNP GPTGTLGAEGLQGPQGSQGNPGRKGEKGSQGQKGPQGSPGLMGAKGSTGRPGLLGKKGEPGLPGDLGPVGQTGQ RGRQGDSGIPGYGQMGRKGVKGPRGFPGDAGQK, that are found to demonstrate repeat occurrences of core peptide sequences flanked by R or K repeats and from which desirable peptide modulators of mTOR may be selected. Another angiogenic protein of which a peptide is provided herein is human alpha-fetoprotein wherein a peptide with sequence KDLCQAQGVALQTMK is observed, comprising an antigenic core QAQGVALQ, and derivatives AQGV, AQGVA, AQGVAL, QGVALQ and GVALQ are found, all useful as autophagy inhibiting peptide modulator of mTOR, preferably in combination with rapid- or short-acting insulins. Typically, autophagy-inhibition by dipeptide modulator of mTOR AQ has recently been demonstrated in piglets, having improved modulating characteristics over amino acids A+Q (Zhang et al., Alanyl-glutamine supplementation regulates mTOR and ubiquitin proteasome proteolysis signaling pathways in piglets. Nutrition. 2016 October; 32 (10): 1123-31.).

On the other hand, provided is a growth factor formulation (formulations herein preferably comprising pharmaceutical formulations or pharmaceutical compositions), such as an insulin formulation, an IGF formulation, a PDGF formulation, a VEGF formulation, or a formulation of another growth factor useful in treatment of vascular disease, in particular useful in glycemic control, having been provided with an autophagy inhibiting peptide-modulator of mTOR for the purposes of glycemic control, as provided herein. A formulation of a growth factor and an autophagy inhibiting modulator of mTOR is provided, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and valine (V). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and leucine (L). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), leucine (L), and proline (P). In a preferred embodiment, provided is an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), valine (V), leucine (L), and proline (P). Such autophagy inhibiting peptide-modulators of mTOR and use thereof in growth factor formulations, as provided herein, typically comprise peptides and/or sources of amino acids that target the nutrient--in particular the amino acid--sensing system of the mechanistic target of rapamycin, mTOR, inhibit autophagy, and therewith induce activities in a patient in need thereof. Typically, in the case of glycemic control through the use of insulin formulations, insulin requirements (units/kg/day) are determined on the basis of patient's age, weight, and residual pancreatic insulin activity. Patients will typically require a total daily insulin dose of 0.4-1.0 units/kg/day and typical starting dose in metabolically-stable patients is 0.5 units/kg/day. However, as said, glycemic control, even with insulin, is no substitute for C-peptide deficiency.

Typically, herein peptides are defined as having 50 or less amino acids, for the purpose of this disclosure, proteins are defined as having >50 amino acids. A autophagy inhibiting peptide-modulator of mTOR herein is defined as a linear, branched or circular string of no longer than 50 amino acids that comprises a peptide sequence with at least 50%, more preferably at least 75%, most preferably 100% amino acids selected from the group of autophagy inhibiting amino acids alanine (in one letter code: A), glutamine (Q), glycine (G), valine (V), leucine (L), proline (P), isoleucine (I), arginine (R) and asparagine (N). Molecular mode-of-action (MoA) of this group of peptides does not depend on their exact sequence. Instead, their constituent amino acids provide autophagy inhibiting signals to the nutrient-sensing system of mTOR; leading to inhibition of autophagy and resulting in proteogenesis with resolve of disease.

As reviewed in Sciarretta et al (New Insights into the Role of mTOR Signaling in the Cardiovascular System. Circ Res. 2018 Feb. 2;122 (3): 489-505) the mechanistic (previously called mammalian) target of rapamycin (mTOR) is an atypical serine/threonine kinase, belonging to the phosphoinositide kinase-related kinase (PIKK) family. It is an evolutionarily conserved protein that plays a central role in the regulation of cellular physiology, metabolism and stress responses Some call it the cellular master switch. mTOR interacts with specific adaptor proteins and forms two distinct macromolecular complexes, named mTOR complex 1 (mTORC1) and mTOR complex 2 (mTORC2). The previous paradigm in mTOR biology included involvement of mTOR in the regulation of protein synthesis, cellular growth and ribosomal biogenesis, and sensing and integrating different upstream inputs, such as growth factors, nutrients, amino acids, starvation and hypoxia. However, it is now known that the mTOR pathway also controls other important cellular processes, including cell survival, mitochondrial biogenesis and function, lipid synthesis and autophagy. The signaling network of mTORC2 is less characterized than that of mTORC1. Some studies have suggested that the activities of mTORC1 and mTORC2 are strongly interconnected, with mTORC2 being less sensitive to acute rapamycin treatment than mTORC1. Previous work also indicated that mTORC2 is involved in the regulation of cell survival, growth and proliferation, and controls cell architecture and polarity. Typically, amino acids are not only the building blocks of protein, but are also signaling molecules, as well as regulators of gene expression, metabolic processes, and developmental changes in the body, with crucial roles of amino acids and their metabolites in the health and disease. Substantial evidence indicates that amino acids play a fundamental role in the vascular system. While amino acids serve as basic building blocks for protein synthesis and constitute an important energy source, a select group has been widely studied in the context of vascular disease. mTOR, in particular mTORC1, is a critical kinase that regulates cell growth and proliferation, by sensing nutrients such as amino acids and glucose. Alanine and glutamine are the most abundant amino acids circulating in the blood. Amino acids not only participate in intermediary metabolism but also stimulate insulin-mechanistic target of rapamycin (MTOR)-mediated signal transduction which controls the major metabolic pathways. Among these is the pathway of autophagy which takes care of the degradation of long-lived proteins and of the elimination of damaged or functionally redundant organelles. Proper functioning of this process is essential for cell survival. Dysregulation of autophagy has been implicated in the etiology of several pathologies. Clearly, only certain amino acids are able to modulate autophagy, and their functions are highly cell-specific.

Plasmodium findings identify VGVAPG(n)-motif instrumental in generating insulin resistance in primates, among which humans.

As discussed by Eze et al. (Asymptomatic Plasmodium infection and glycemic control in adults: Results from a population-based survey in south-central Cote d′Ivoire, Diabetes Research and Clinical Practice, Volume 156, 2019, 107845), inflammatory responses to repeated infections with malaria parasites (Plasmodium species) may lead to sustained physiological changes in insulin sensitivity that increase diabetes susceptibility. Plasmodium infection (PI) induces systemic inflammatory pathways which are also characteristic of type 2 diabetes. Pro-inflammatory blood markers such as circulating C-reactive protein, IL-1B, IL-6, IL-8, and IL-10 were associated with both malaria severity and insulin resistance. IL-6 was higher in adults from a malaria-endemic setting than in their age-adjusted western counterparts. In utero malaria is thought to direct fetal physiological pathways toward insulin resistance and impaired fasting glucose (FG) in adolescence.

Abidha, C.A., Amoako, Y.A., Nyamekye, R. K. et al. (Fasting blood glucose in a Ghanaian adult is causally affected by malaria parasite load: a mechanistic case study using convergent cross mapping. Malar J 21, 93 (2022)), executed a study aimed at verifying the hypothesis that increased fasting blood glucose (FG) promotes parasite growth as reflected by increased parasite density. However, the reciprocal was found. Seven adults without DM were recruited in rural Ghana to determine the relationships between FG and malaria parasite load. Socio-economic data were recorded in questionnaire-based interviews. Over a period of 6 weeks, FG and Plasmodium sp. infection were measured in peripheral blood samples photometrically and by polymerase chain reaction (PCR)-assays, respectively. Daily physical activity and weather data were documented via smartphone recording. For the complex natural systems of homeostatic glucose control and Plasmodium sp. life cycle, empirical dynamic modelling was applied. At baseline, four men and three women (median age, 33 years; interquartile range, 30-48) showed a median FG of 5.5 (5.1-6.0 mmol/L); one participant had an asymptomatic Plasmodium sp. infection (parasite density: 240/μL). In this participant, convergent cross mapping (CCM) for 34 consecutive days, showed that FG (and thus insulin resistance) was causally affected by parasite density (p<0.02), while the reciprocal relationship was not discernible (p >0.05).

Beyond the above embodiments further embodiments are disclosed here below.

### Further Embodiment 1

A method for treatment of a human subject deemed having episodes of hypoglycemia associated with hyperinsulinemia comprising treating said subject with a modulator or pharmaceutical composition provided with a molecule carrying an elastin receptor binding motif that is at least functionally equivalent to a PG-domain peptide with motif XGXXPG wherein G stands for the amino acid glycine, P for proline and X for any amino acid.

### Further Embodiment 2

A method for treatment of a human subject deemed having episodes of hypoglycemia associated with hyperinsulinemia comprising treating said subject with a modulator or pharmaceutical composition provided with a molecule carrying a Na⁺K⁺ATPase activating motif that is at least functionally equivalent to a pentapeptide domain peptide with motif EXXXQ wherein E stands for the amino acid glutamate, G for glutamine and X for any amino acid.

### Further Embodiment 3

A method for treatment of a human subject deemed having episodes of hypoglycemia associated with hyperinsulinemia comprising treating said subject with a modulator or pharmaceutical composition provided with a molecule carrying an elastin receptor binding motif that is at least functionally equivalent to a PG-domain peptide with motif XGXXPG wherein G stands for the amino acid glycine, P for proline and X for any amino acid and further provided with a molecule carrying a Na⁺K+ ATP-ase activating motif that is at least functionally equivalent to pentapeptide domain motif EXXXQ wherein E stands for the amino acid glutamate, G for glutamine and X for any amino acid.

### Further Embodiment 4

A method according to further embodiments 1 or 3 wherein said molecule comprises a peptide with motif selected from the group XGXXPG, xGXXPG, xGxXPG, xGXxPG and xGxxPG.

### Further Embodiment 5

A method according to further embodiment 4 wherein said molecule comprises a peptide with motif selected from the group of peptides at least functionally equivalent to any of a peptide motif VGVAPG, vGVAPG, vGvAPG, vGVaPG, vGvaPG, LGGGPG, IGGGPG, PGAYPG, pGAYPG, pGaYPG, pGAyPG or pGayPG, AQGVAPG(n), gpavgqa(n), LQGVAPG(n), gpavgql(n),.

### Further Embodiment 6

A method according to further embodiment 5 wherein said peptide is selected from the group VGVAPGVGVAPGVGVAPG, QVGQVELGGGPGAGSLQP and GAYPGAPGAYPGAPAPGV or a functional equivalent thereof.

### Further Embodiment 7

A method according to further embodiments 1 or 3 wherein said molecule comprises a peptide with motif selected from the group gpxxgx, gpxxgX, gpxXgX, gpXxgX and gpXXX.

Further Embodiment 8. A method according to further embodiment 7 wherein said molecule comprises a peptide with motif selected from the group at least functionally equivalent to any of a peptide motif gpavgv, gpavgV, gpaVgV, gpAvgV, gpAVgV, gpgggI, gpgggL, gpyagp, gpaygP, gpaYgP, gpAygP or gpAYgP.

### Further Embodiment 9

A method according to further embodiment 8 wherein said peptide is selected from the group gpavgvgpavgvgpavgv, pqlsgagpgggIevqgvq, vgpapagpyagpagpyay or a functional equivalent thereof.

### Further Embodiment 10

A method according to further embodiments 2 or 3 wherein said molecule comprises a peptide with motif selected from the group EXXXQ, ExXXQ, EXXXQ, EXXxQ, ExxXQ, EXxxQ, ExXxQ and ExxxQ.

### Further Embodiment 11

A method according to further embodiment 10 wherein said peptide is selected from the group EGSLO, EVPPQ, EGALQ, EGPLQ, EVAQQ, EGPPQ, EAPLO, EMALO, EVARQ, or a functional equivalent thereof.

### Further Embodiment 12

A method according to further embodiments 2 or 3 wherein said molecule comprises a peptide with motif selected from the group qxxxe, qxxXe, qxXxe, qXxxe, qxxXq, qXXxe, qXxXe and qXXXe. Further embodiment 13

A method according to further embodiment 12 wherein said peptide is selected from the group qlsge, qppve, qlage, qlpge, qqave, qppge, qlpae, qlame, qrave or a functional equivalent thereof.

### Further Embodiment 14

A method according to anyone of further embodiment 4 to 13 wherein said peptide is provided with improved resistance to proteolytic degradation by providing each of the N- and C-terminal flanks of said peptide with up to three D-amino acids.

### Further Embodiment 15

A method according to further embodiment 14 wherein said peptide is selected from the synthesis.

### Further Embodiment 16

A method according to anyone of further embodiment 1 to 15 wherein said hyperinsulinemia is endogenous hyperinsulinemia.

### Further Embodiment 17

A method according to anyone of further embodiment 1 to 16 wherein said hyperinsulinemia comprises congenital hyperinsulinemia.

### Further Embodiment 18

A method according to anyone of further embodiments 1 to 17 wherein said treatment comprises modulating beta-cell membrane polarization.

### Further Embodiment 19

A method according to anyone of further embodiment 1 to 18 wherein said treatment comprises modulating NA+K⁺ATPase activity.

### Further Embodiment 20

A method according to anyone of further embodiment 1 to 19 wherein said treatment comprises modulating insulin secretion.

### Further Embodiment 21

A molecule carrying an elastin receptor binding motif that is at least functionally equivalent to a PG-domain peptide motif XGXXPG wherein G stands for the amino acid glycine, P for proline and X for any amino acid for use in the treatment of a human subject deemed having episodes of hypoglycemia associated with hyperinsulinemia.

### Further Embodiment 22

A molecule carrying a Na⁺K⁺ATPase activating motif that is at least functionally equivalent to a pentapeptide domain peptide motif EXXXQ wherein E stands for the amino acid glutamate, G for glutamine and X for any amino acid for use in the treatment of a human subject deemed having episodes of hypoglycemia associated with hyperinsulinemia.

### Further Embodiment 23

A molecule carrying an elastin receptor binding motif that is at least functionally equivalent to a PG-domain peptide motif XGXXPG wherein G stands for the amino acid glycine, P for proline and X for any amino acid and carrying a Na⁺K⁺ATPase activating motif that is at least functionally equivalent to a pentapeptide domain peptide motif EXXXQ wherein E stands for the amino acid glutamate, G for glutamine and X for any amino acid for use in the treatment of a human subject deemed having episodes of hypoglycemia associated with hyperinsulinemia.

Further Embodiment 24. A molecule according to further embodiments 21 and 23 wherein said molecule is a peptide with motif selected from the group XGXXPG, xGXXPG, xGxXPG, xGXxPG and xGxxPG.

### Further Embodiment 25

A molecule according to further embodiment 24 wherein said molecule is a peptide with motif selected from the group at least functionally equivalent to any of a peptide motif VGVAPG, vGVAPG, vGvAPG, vGVaPG, vGvaPG, LGGGPG, IGGGPG, PGAYPG, pGAYPG, pGaYPG, pGAyPG or pGayPG.

### Further Embodiment 26

A molecule according to further embodiment 24 wherein said peptide is selected from the group VGVAPGVGVAPGVGVAPG, QVGQVELGGGPGAGSLQP and GAYPGAPGAYPGAPAPGV or a functional equivalent thereof.

### Further Embodiment 27

A molecule according to further embodiments 21 and 23 wherein said molecule is a peptide with motif selected from the group gpxxgx, gpxxgX, gpxXgX, gpXxgX and gpXXgX.

### Further Embodiment 28

A molecule according to further embodiment 27 wherein said molecule is a peptide with motif selected from the group at least functionally equivalent to any of a peptide motif gpavgv, gpavgV, gpaVgV, gpAvgV, gpAVgV, gpgggI, gpgggL, gpyagp, gpaygP, gpaYgP, gpAygP or gpAYgP.

### Further Embodiment 29

A molecule according to further embodiments 22 and 23 wherein said molecule comprises a peptide with motif selected from the group EXXXQ, ExXXQ, EXXXQ, EXXxQ, ExxXQ, EXxxQ, ExXxQ and ExxxQ.

### Further Embodiment 30

A method according to further embodiment 29 wherein said peptide is selected from the group EGSLO, EVPPQ, EGALQ, EGPLQ, EVAQQ, EGPPQ, EAPLO, EMALQ, EVARQ, or a functional equivalent thereof.

### Further Embodiment 31

A molecule according to further embodiments 22 and 23 wherein said molecule comprises a peptide with motif selected from the group qxxxe, qxxXe, qxXxe, qXxxe, qxXXq, qXXxe, qXxXe and qXXXe.

### Further Embodiment 32

A method according to further embodiment 31 wherein said peptide is selected from the group qlsge, qppve, qlage, qlpge, qqave, qppge, qlpae, qlame, qrave or a functional equivalent thereof.

### Further Embodiment 33

A molecule according to anyone of further embodiment 24 to 32 wherein said peptide is provided with improved resistance to proteolytic degradation by providing each of the N- and C-terminal flanks of said motif with up to three D-amino acids.

### Further Embodiment 34

A molecule according to further embodiment 33 wherein said peptide is selected from the group vGVAPGVGVAPGVGVApG, vGVAPGVGVAPGEGSLQa, qVGQVELGGGPGAGSLQp, qVGQVELGGGPGEGSLQa, GaYPGAPGAYPGEGSLQav, GaYPGAPGAYPGAPAPGv, vGvAPGVGVAPGVGVapG, qvGQVELGGGPGAGSLqp, GayPGAPGAYPGAPApGv vGvaPGVGVAPGVGvapG, qvGqVELGGGPGAGSIqp and GaypGAPGAYPGAPapgV, gpavgvgpavgvgpavgv, pqlsgagpgggIevqgvq, vgpapagpyagpagpyay, aqlsgegpavgvgpavgv, aqlsgegpgggIevqgvq, aqlsgegpyagpagpyay, vGVAPGVGVApG, vGVAPGEGSLQa, qGGGPGAGSLQp, qGGGPGEGSLQa, GaAYPGEGSLQav, GaYPGAAPAPGv, vGvAPGVGVapG, qvGGGPGAGSLqp, GayPGAAPApGv vGvaPGVGvapG, qvGqVELGGGPGqp and GaypGAAPapgV, gpavgvgpavgv, pqlsgegpgggIvq, vgpapgpagpyay, aqlsgegpavgv, aqlsgegpggqgvq, aqlsgegpagpyay, or a functional equivalent thereof.

### Further Embodiment 35

A molecule according to anyone of further embodiment 21 to 34 wherein said hyperinsulinemia is endogenous hyperinsulinemia.

### Further Embodiment 36

A molecule according to further embodiment 35 wherein said hyperinsulinemia comprises congenital hyperinsulinemia.

### Further Embodiment 37

A molecule according to anyone of embodiments 21 to 36 obtainable by peptide synthesis.

### Further Embodiment 38

A molecule according to any one of embodiments 21 to 37 comprising at least one D-amino acid, preferably at least 2, more preferably at least 3 D-amino acids.

### Further Embodiment 39

A modulator or pharmaceutical composition comprising a molecule according to any of embodiments 21 to 38.

### Further Embodiment 40

A composition according to further embodiment 39 comprising a pharmaceutically acceptable excipient.

### Further Embodiment 41

A cell provided with a molecule according to anyone of embodiments 21 to 38.

### Further Embodiment 42

A cell according to further embodiment 41 provided with an elastin-receptor-complex (ERC).

### Further Embodiment 43

A cell according to further embodiment 41 or 42 provided with an insulin receptor kinase (IRK).

### Further Embodiment 44

A cell according to any of further embodiment 41 to 43 provided with DANA.

### Further Embodiment 45

A cell culture comprising at least one cell according to anyone of embodiments 41 to 44.

### Further Embodiment 46

Use of a cell culture according to further embodiment 45 for screening a molecule and determining said molecule's effect on modulating NA+K⁺ATPase activity.

### Further Embodiment 47

Use of a cell culture according to further embodiment 45 for screening a molecule and determining said molecule's effect on membrane polarization.

### Further Embodiment 48

Use of a cell culture according to further embodiment 45 for screening a molecule and determining said molecule's effect on modulating insulin secretion.

### Further Embodiment 49

A method for identifying a source of, preferably L-proteinogenic, amino acids, preferably a peptide, capable of inducing increased glycaemia in prevention or treatment of a hyperinsulinism of a subject, comprising providing cells with a peptide comprising L-proteinogenic amino acids, said amino acids for least 50% selected from the group of alanine (in one letter code: A), glutamine (Q), glycine (G), valine (V), leucine (L), isoleucine (I), proline (P) and arginine (R), and determining angiogenic activity in a sprouting assay.

### Further Embodiment 50

A method for inducing increased glycaemia in prevention or treatment of a hyperinsulinism of a subject, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P) and arginine (R).

### Further Embodiment 51

A method for inducing increased glycaemia in prevention or treatment of a hyperinsulinism of a subject, comprising providing cells with a peptide as a source of, preferably L-proteinogenic, amino acids, said peptide consisting of amino acids that are for at least 50% selected from the group of alanine (in one letter code: A), glutamine (Q), glycine (G), valine (V), leucine (L), isoleucine (I), proline (P) and arginine (R).

### Further Embodiment 52

A method according to further embodiment 50 or 51, wherein said source of, preferably L-proteinogenic, amino acids, preferably a peptide, is identifiable with a method according to further embodiment 49.

### Further Embodiment 53

A method according to anyone of further embodiments 49 to 52 wherein said amino acids are for at least 50% selected from the group of alanine (in one letter code: A), glutamine (Q), glycine (G), valine (V), leucine (L), and proline (P).

### Further Embodiment 54

A method according to anyone of further embodiments 50 to 53 wherein said source or peptide has an amino acid sequence that is derived from a peptide or protein shown or suspected of having activities.

### Further Embodiment 55

A method according to further embodiment 54 wherein said peptide has an amino acid sequence that is derived from a chorionic gonadotropin.

### Further Embodiment 56

A method according to further embodiment 54 wherein said peptide has an amino acid sequence that is derived from a C-peptide.

### Further Embodiment 57

A method according to further embodiment 54 wherein said peptide has an amino acid sequence that is derived from a galectin-3.

### Further Embodiment 58

A method according to further embodiment 54 wherein said peptide has an amino acid sequence that is derived from a luteotropin.

### Further Embodiment 59

A method according to further embodiment 54 wherein said peptide has an amino acid sequence that is derived from an extra-cellular matrix protein.

### Further Embodiment 60

A method according to further embodiment 59 wherein said extra-cellular matrix protein is an elastin or collagen.

### Further Embodiment 61

A method according any one of further embodiments 49 to 60 wherein said peptide comprises less than 31 amino acids, preferably less than 26 amino acids, more preferably less than 21 amino acids, more preferably less than 16 amino acids, more preferably less than 13 amino acids.

### Further Embodiment 62

A method according any one of further embodiments 49 to 61 wherein said peptide comprises more than 3 amino acids, preferably more than 5 amino acids, more preferably more than 7 amino acids, more preferably at least 9 amino acids.

### Further Embodiment 63

A method according to further embodiment 62 wherein said source comprises a peptide that comprises more than 6 amino acids and less than 26 amino acids.

### Further Embodiment 64

A method according to further embodiment 62 wherein said source comprises a peptide that comprises more than 7 amino acids and less than 31 amino acids.

### Further Embodiment 65

A method according to any one of further embodiments 49 to 64 wherein said peptide is provided as a salt of an organic acid, preferably wherein said peptide is provided as a salt of maleic acid, more preferably of acetic acid, more preferably of tartaric acid and most preferably of citric acid.

### Further Embodiment 66

A method according to any one of further embodiments 49 to 65 wherein said cells are of human origin.

### Further Embodiment 67

A method according to any one of further embodiments 49 to 66 wherein said peptide is of human origin.

### Further Embodiment 67

A further embodiment according to embodiments 49 to 66 wherein said peptide is provided as a salt of an organic acid, preferably wherein said peptide is provided as a salt of maleic acid, more preferably of acetic acid, more preferably of tartaric acid and most preferably of citric acid.

### Further Embodiment 68

A method for treatment of a human subject deemed having episodes of hypoglycemia associated with hyperinsulinemia comprising treating said subject with a modulator or pharmaceutical composition provided with a molecule carrying an elastin receptor binding motif that is at least functionally equivalent to a peptide motif XGXXPG wherein G stands for the amino acid glycine, P for proline and X for any amino acid.

### Further Embodiment 69

A method for treatment of a human subject deemed having episodes of hypoglycemia associated with hyperinsulinemia comprising treating said subject with a modulator or pharmaceutical composition provided with a molecule carrying a Na⁺K⁺ATPase activating motif that is at least functionally equivalent to a pentapeptide domain peptide motif EXXXQ wherein E stands for the amino acid glutamate, G for glutamine and X for any amino acid.

### Further Embodiment 70

A method for treatment of a human subject deemed having episodes of hypoglycemia associated with hyperinsulinemia comprising treating said subject with a modulator or pharmaceutical composition provided with a molecule carrying an elastin receptor binding motif that is at least functionally equivalent to a PG-domain peptide motif XGXXPG wherein G stands for the amino acid glycine, P for proline and X for any amino acid and further provided with a molecule carrying a Na⁺K⁺ATP-ase activating motif that is at least functionally equivalent to peptapeptide domain motif EXXXQ wherein E stands for the amino acid glutamate, G for glutamine and X for any amino acid.

### Further Embodiment 71

A method of treating a subject diagnosed as having episodes of hypoglycemia associated with hyperinsulinemia, the method comprising:
a. administering to the subject a modulator or pharmaceutical composition comprising a molecule having an elastin receptor binding motif that is at least functionally equivalent to a peptide motif XGXXPG, wherein G stands for glycine, P stands for proline, and X stands for any amino acid, so as to reduce the episodes of hypoglycemia associated with hyperinsulinemia.

### Further Embodiment 72

A method of treating a subject diagnosed as having episodes of hypoglycemia associated with hyperinsulinemia, the method comprising:
b. administering to the subject a modulator or pharmaceutical composition comprising a molecule having a Na⁺K⁺ATPase activating motif that is at least functionally equivalent to a pentapeptide domain peptide motif EXXXQ, wherein E stands for glutamate, G stands for glutamine, and X stands for any amino acid, so as to reduce the episodes of hypoglycemia associated with hyperinsulinemia.

Further Embodiment 73. A method of treating a subject diagnosed as having episodes of hypoglycemia associated with hyperinsulinemia, the method comprising:
c. administering to the subject a modulator or pharmaceutical composition comprising:
   i. a molecule having an elastin receptor binding motif that is at least functionally equivalent to a PG-domain peptide motif XGXXPG, wherein G stands for glycine, P stands for proline, and X stands for any amino acid, and
   ii. a molecule carrying a Na⁺K⁺ATP-ase activating motif that is at least functionally equivalent to peptapeptide domain motif EXXXQ, wherein E stands for glutamate, G stands for glutamine and X stands for any amino acid, so as to reduce the episodes of hypoglycemia associated with hyperinsulinemia.
   iii.

### Further Embodiment 74

A method for identifying an amino acid motif that binds to a cellular receptor of a mammal, wherein the method comprises the following steps in consecutive order:
(i) selecting candidate amino acid motifs;
(ii) determining the frequency of occurrence of each of the candidate amino acid motifs in the proteome of a pathogen; wherein the pathogen is a pathogen that can infect the mammal;
and
iii) identifying the candidate amino acid motif with the highest frequency of occurrence in the proteome of the pathogen as the amino acid motif that binds to the cellular receptor of the mammal.

### Further Embodiment 75

The method according to further embodiment 74, wherein the mammal is a primate. The method according to further embodiments 74 or 75, wherein the mammal is a human.

### Further Embodiment 76

The method according to any one of embodiments 73 to 75, wherein the cellular receptor is a receptor that is expressed in immune cells.

### Further Embodiment 77

The method according to any one of embodiments 73 to 75, wherein the cellular receptor is the elastin receptor complex.

### Further Embodiment 78

The method according to any one of embodiments 73 to 77, wherein in step (i) candidate amino acid motifs are selected based on known characteristics of the amino acid motif that binds to the cellular receptor of the mammal.

### Further Embodiment 79

The method according to embodiment 78, wherein the known characteristics are one or more known amino acid(s) of the amino acid motif that binds to the cellular receptor of the mammal.

### Further Embodiment 80

The method according to embodiment 78, wherein the known characteristics are a known secondary structure of the amino acid motif that binds to the cellular receptor of the mammal.

### Further Embodiment 81

The method according to any one of embodiments 73 to 80, wherein in step (i) at least 100 candidate amino acid motifs are selected, preferably wherein in step (i) at least 200 candidate amino acid motifs are selected.

### Further Embodiment 82

The method according to any one of embodiments 78 to 81, wherein the cellular receptor is the elastin receptor complex, and wherein the candidate amino acid motifs comprise the amino acid sequence xGxxPx, wherein G is glycine, P is proline, and x is any natural amino acid.

### Further Embodiment 83

The method according to any one of embodiments 78 to 81, wherein the cellular receptor is the elastin receptor complex, and wherein the candidate amino acid motifs comprise the amino acid sequence xGxxPG, wherein G is glycine, P is proline, and x is any natural amino acid.

### Further Embodiment 84

The method according to any one of embodiments 78 to 81, wherein the cellular receptor is the elastin receptor complex, and wherein the candidate amino acid motifs are all of the amino acid motifs that have the amino acid sequence XGXXPG, wherein G is glycine, P is proline, and X is any natural amino acid.

### Further Embodiment 85

The method according to any one of embodiments 73 to 85, wherein the pathogen is a virus, a bacterium, or a protozoon.

### Further Embodiment 86

The method according to any one of embodiments 73 to 186, wherein the pathogen is a pathogen that acquires host proteins during natural infection.

### Further Embodiment 87

The method according to any one of embodiments 73 to 76, wherein the pathogen is Plasmodium spp.

### Further Embodiment 88

The method according to embodiment 87, wherein the pathogen is primate-infecting or rodent-infecting

Plasmodium spp.

### Further Embodiment 89

The method according to embodiment 88, wherein the primate-infecting Plasmodium spp. is one or more Plasmodium spp. selected from the list consisting of Plasmodium reichenowi, Plasmodium malariae, Plasmodium gonderi, Plasmodium vivax, Plasmodium ovale, Plasmodium falciparum, Plasmodium coatneyi, Plasmodium sp. DRC-Itaito, and Plasmodium sp. Gorilla clade.

### Further Embodiment 90

The method according to any one of embodiments 73 to 90, wherein the proteome of the pathogen is a partial proteome of the pathogen.

### Further Embodiment 91

The method according to embodiment 90, wherein the partial proteome of the pathogen comprises the cell membrane proteins of the pathogen and the secreted proteins of the pathogen.

### Further Embodiment 92

The method according to any one of embodiments 73 to 91, wherein the pathogen is primate-infecting Plasmodium spp., and wherein the proteome of the pathogen is a partial proteome comprising the blood-stage proteins of primate-infecting Plasmodium spp.

What is claimed:

1-15. (canceled)

16. A modulator of insulin-insulin receptor signaling for use in the treatment of a mammalian subject deemed having episodes of hypoglycemia associated with hyperinsulinemia in congenital hyperinsulinemia, wherein said modulator is provided with a molecule carrying an elastin receptor binding motif.

17. The modulator for use according to claim 16, wherein said subject is a primate or a rodent.

18. The modulator for use according to claim 16, wherein said modulator is capable of reducing insulin-induced translocation of glucose transporter 4 (GLUT4) to the plasma membrane of a fat- or muscle-cell of said subject.

19. The modulator for use according to claim 16, wherein said modulator is provided with a molecule carrying an elastin receptor binding motif that is at least functionally equivalent to a peptide XGXXPG wherein G stands for the amino acid glycine, P for proline and X for any amino acid.

20. The modulator for use according to claim 16, wherein said modulator comprises a peptide or peptidomimetic carrying an elastin receptor binding motif that is at least functionally equivalent to a peptide XGXXPG wherein G stands for the amino acid glycine, P for proline and X for any amino acid.

21. The modulator for use according to claim 20, wherein said peptide with said motif is selected from the group of peptides at least functionally equivalent to any of a peptide VGVAPG(n), vGVAPG(n), vGvAPG(n), vGVaPG(n), vGvaPG(n), gpavgv(n) PGAVPG(n), pGvAPG(n), pGVaPG(n), pGvaPG(n), gpvagp(n), LGGGPG(n), IGGGPG(n), gpgggI(n), PGAYPG(n), pGAYPG(n), pGaYPG(n), pGAyPG(n), pGayPG(n), or gpyagp(n), AQGVAPG(n), gpavgqa(n), LQGVAPG(n), gpavgql(n), wherein lowercase letters indicate D-amino acids, whereas uppercase letters indicate L-amino acids, with the notable exception of glycine, wherein both g and/or G indicate the amino acid glycine and wherein(n) indicates the number of occurrences of said motif and wherein(n) varies from 1 to 8, allowing targeting of the modulator to the elastin receptor.

22. The modulator for use according to claim 20, wherein said peptide with said motif is selected from the group of peptides at least functionally equivalent to any of a peptide VGVAPG(n), gpavgv(n) PGAVPG(n), gpvagp(n) wherein lowercase letters indicate D-amino acids, whereas uppercase letters indicate L-amino acids, with the notable exception of glycine, wherein both g and/or G indicate the amino acid glycine and wherein(n) indicates the number of repeat occurrences of said motif and wherein (n) varies from 1 to 8, allowing targeting of the modulator to the elastin receptor.

23. The modulator for use according to claim 20, wherein said peptide with said motif is at least functionally equivalent to a peptide VGVAPGVGVAPGVGVAPG, QVGQVELGGGPGAGSLQP or GAYPGAPGAYPGAPAPGV.

24. The modulator for use according to claim 20, wherein said peptide with said motif is at least functionally equivalent to a peptide VGVAPGVGVAPGVGVAPG or AVPGAVPGAVPG.

25. A pharmaceutical composition comprising the modulator as defined in any claim 16.

26. The pharmaceutical composition according to claim 25, formulated for parenteral application.

27. The pharmaceutical composition according to claim 25, formulated for oral application.

28. A method for treatment of a mammalian subject deemed having episodes of hypoglycemia associated with hyperinsulinemia in congenital hyperinsulinemia, comprising administering to said subject the modulator as defined in claim 16.

29. A method for treatment of a mammalian subject deemed having episodes of hypoglycemia associated with hyperinsulinemia in congenital hyperinsulinemia, comprising administering to said subject a pharmaceutical composition according to claim 25.

30. The method according to claim 28, wherein said subject is a primate or a rodent.

31. The method according to claim 29, wherein said subject is a primate or a rodent.


SEQUENCE LISTING (330 sequences)

SEQ ID NO: 1 - protein, 8 aa, synthetic construct
  SITE 2: note = D-Alanine
  SITE 4: note = D-Proline
  SITE 8: note = D-Leucine
GAGPGGGL

SEQ ID NO: 2 - protein, 8 aa, synthetic construct
  SITE 2: note = D-Alanine
  SITE 4: note = D-Proline
  SITE 7: note = D-Alanine
  SITE 8: note = D-Leucine
GAGPGGAL

SEQ ID NO: 3 - protein, 6 aa, synthetic construct
  SITE 1: note = D-Alanine
  SITE 3: note = D-Proline
  SITE 6: note = D-Leucine
AGPGGL

SEQ ID NO: 4 - protein, 7 aa, synthetic construct
  SITE 2: note = D-Proline
  SITE 6: note = D-Proline
  SITE 7: note = D-Alanine
GPGGGPA

SEQ ID NO: 5 - protein, 6 aa, synthetic construct
  SITE 2: note = D-Proline
  SITE 6: note = D-Leucine
  SITE 5: note = D-Alanine
GPGGAL

SEQ ID NO: 6 - protein, 5 aa, synthetic construct
  SITE 2: note = D-Proline
GPGGG

SEQ ID NO: 7 - protein, 5 aa, synthetic construct
  SITE 2: note = D-Proline
  SITE 4: note = D-Alanine
GPGAG

SEQ ID NO: 8 - protein, 6 aa, synthetic construct
  SITE 2: note = D-Proline
  SITE 3: note = D-Valine
  SITE 4: note = D-Alanine
  SITE 6: note = D-Proline
GPVAGP

SEQ ID NO: 9 - protein, 6 aa, synthetic construct
  SITE 2: note = D-Proline
  SITE 3: note = D-Alanine
  SITE 4: note = D-Valine
  SITE 6: note = D-Valine
GPAVGV

SEQ ID NO: 10 - protein, 6 aa, synthetic construct
VGVAPG

SEQ ID NO: 11 - protein, 5 aa, synthetic construct
GGGPG

SEQ ID NO: 12 - protein, 5 aa, synthetic construct
GAGPG

SEQ ID NO: 13 - protein, 18 aa, synthetic construct
VGVAPGVGVAPGVGVAPG

SEQ ID NO: 14 - protein, 18 aa, synthetic construct
QVGQVELGGGPGAGSLQP

SEQ ID NO: 15 - protein, 18 aa, synthetic construct
GAYPGAPGAYPGAPAPGV

SEQ ID NO: 16 - protein, 18 aa, synthetic construct
VGVAPGVGVAPGVGVAPG

SEQ ID NO: 17 - protein, 12 aa, synthetic construct
AVPGAVPGAVPG

SEQ ID NO: 18 - protein, 6 aa, synthetic construct
PGAVPG

SEQ ID NO: 19 - protein, 4 aa, synthetic construct
AVPG

SEQ ID NO: 20 - protein, 4 aa, synthetic construct
  SITE 2: note = D-Proline
  SITE 3: note = D-Valine
  SITE 4: note = D-Alanine
GPVA

SEQ ID NO: 21 - protein, 6 aa, synthetic construct
  SITE 2: note = D-Proline
  SITE 3: note = D-Alanine
  SITE 4: note = D-Valine
  SITE 6: note = D-Valine
GPAVGV

SEQ ID NO: 22 - protein, 18 aa, synthetic construct
VGVAPGVGVAPGVGVAPG

SEQ ID NO: 23 - protein, 12 aa, synthetic construct
AVPGAVPGAVPG

SEQ ID NO: 24 - protein, 18 aa, synthetic construct
QVGQVELGGGPGAGSLQP

SEQ ID NO: 25 - protein, 18 aa, synthetic construct
GAYPGAPGAYPGAPAPGV

SEQ ID NO: 26 - protein, 7 aa, synthetic construct
AQGVAPG

SEQ ID NO: 27 - protein, 7 aa, synthetic construct
LQGVAPG

SEQ ID NO: 28 - protein, 18 aa, synthetic construct
  SITE 2: note = D-Proline
  SITE 8: note = D-Proline
  SITE 14: note = D-Proline
  SITE 3: note = D-Alanine
  SITE 9: note = D-Alanine
  SITE 15: note = D-Alanine
  SITE 4: note = D-Valine
  SITE 6: note = D-Valine
  SITE 10: note = D-Valine
  SITE 12: note = D-Valine
  SITE 16: note = D-Valine
  SITE 18: note = D-Valine
GPAVGVGPAVGVGPAVGV

SEQ ID NO: 29 - protein, 12 aa, synthetic construct
  SITE 2: note = D-Proline
  SITE 6: note = D-Proline
  SITE 10: note = D-Proline
  SITE 4: note = D-Alanine
  SITE 8: note = D-Alanine
  SITE 12: note = D-Alanine
  SITE 3: note = D-Valine
  SITE 7: note = D-Valine
  SITE 11: note = D-Valine
GPVAGPVAGPVA

SEQ ID NO: 30 - protein, 18 aa, synthetic construct
  SITE 1: note = D-Proline
  SITE 8: note = D-Proline
  SITE 2: note = D-Glutamine
  SITE 5: note = D-Glutamine
  SITE 18: note = D-Glutamine
  SITE 3: note = D-Leucine
  SITE 12: note = D-Leucine
  SITE 4: note = D-Serine
  SITE 5: note = D-Alanine
  SITE 13: note = D-Glutamic acid
  SITE 14: note = D-Valine
  SITE 17: note = D-Valine
PQLSGAGPGGGLEVQGVQ

SEQ ID NO: 31 - protein, 20 aa, synthetic construct
  SITE 3: note = D-Proline
  SITE 5: note = D-Proline
  SITE 8: note = D-Proline
  SITE 12: note = D-Proline
  SITE 15: note = D-Proline
  SITE 17: note = D-Proline
  SITE 4: note = D-Alanine
  SITE 6: note = D-Alanine
  SITE 10: note = D-Alanine
  SITE 13: note = D-Alanine
  SITE 19: note = D-Alanine
  SITE 1: note = D-Valine
  SITE 9: note = D-Tyrosine
  SITE 18: note = D-Tyrosine
VGPAPAGPYAGPAGPGPYAG

SEQ ID NO: 32 - protein, 7 aa, synthetic construct
  SITE 2: note = D-Proline
  SITE 3: note = D-Alanine
  SITE 7: note = D-Alanine
  SITE 4: note = D-Valine
  SITE 6: note = D-Glutamine
GPAVGQA

SEQ ID NO: 33 - protein, 7 aa, synthetic construct
  SITE 2: note = D-Proline
  SITE 3: note = D-Alanine
  SITE 4: note = D-Valine
  SITE 6: note = D-Glutamine
  SITE 7: note = D-Leucine
GPAVGQL

SEQ ID NO: 34 - protein, 4 aa, synthetic construct
  SITE 2: note = D-Proline
  SITE 3: note = D-Valine
  SITE 4: note = D-Alanine
GPVA

SEQ ID NO: 35 - protein, 6 aa, synthetic construct
  SITE 3: note = D-Alanine
  SITE 2: note = D-Proline
  SITE 4: note = D-Valine
  SITE 6: note = D-Valine
GPAVGV

SEQ ID NO: 36 - protein, 6 aa, synthetic construct
  SITE 1: note = D-Valine
VGVAPG

SEQ ID NO: 37 - protein, 6 aa, synthetic construct
  SITE 1: note = D-Valine
  SITE 3: note = D-Valine
VGVAPG

SEQ ID NO: 38 - protein, 6 aa, synthetic construct
  SITE 1: note = D-Valine
  SITE 4: note = D-Alanine
VGVAPG

SEQ ID NO: 39 - protein, 6 aa, synthetic construct
  SITE 1: note = D-Valine
  SITE 3: note = D-Valine
  SITE 4: note = D-Alanine
VGVAPG

SEQ ID NO: 40 - protein, 6 aa, synthetic construct
  SITE 3: note = D-Alanine
  SITE 2: note = D-Proline
  SITE 4: note = D-Valine
  SITE 6: note = D-Valine
GPAVGV

SEQ ID NO: 41 - protein, 6 aa, synthetic construct
PGAVPG

SEQ ID NO: 42 - protein, 6 aa, synthetic construct
  SITE 1: note = D-Proline
  SITE 3: note = D-Valine
PGVAPG

SEQ ID NO: 43 - protein, 6 aa, synthetic construct
  SITE 1: note = D-Proline
  SITE 4: note = D-Alanine
PGVAPG

SEQ ID NO: 44 - protein, 6 aa, synthetic construct
  SITE 1: note = D-Proline
  SITE 3: note = D-Valine
  SITE 4: note = D-Alanine
PGVAPG

SEQ ID NO: 45 - protein, 6 aa, synthetic construct
  SITE 2: note = D-Proline
  SITE 6: note = D-Proline
  SITE 3: note = D-Alanine
  SITE 4: note = D-Valine
GPAVGP

SEQ ID NO: 46 - protein, 6 aa, synthetic construct
LGGGPG

SEQ ID NO: 47 - protein, 6 aa, synthetic construct
LGGGPG

SEQ ID NO: 48 - protein, 6 aa, synthetic construct
  SITE 2: note = D-Proline
  SITE 6: note = D-Leucine
GPGGGL

SEQ ID NO: 49 - protein, 6 aa, synthetic construct
PGAYPG

SEQ ID NO: 50 - protein, 6 aa, synthetic construct
  SITE 1: note = D-Proline
PGAYPG

SEQ ID NO: 51 - protein, 6 aa, synthetic construct
  SITE 1: note = D-Proline
  SITE 3: note = D-Alanine
PGAYPG

SEQ ID NO: 52 - protein, 6 aa, synthetic construct
  SITE 1: note = D-Proline
  SITE 4: note = D-Tyrosine
PGAYPG

SEQ ID NO: 53 - protein, 6 aa, synthetic construct
  SITE 4: note = D-Tyrosine
  SITE 3: note = D-Alanine
  SITE 1: note = D-Proline
PGAYPG

SEQ ID NO: 54 - protein, 6 aa, synthetic construct
  SITE 1: note = D-Proline
  SITE 6: note = D-Proline
  SITE 3: note = D-Tyrosine
  SITE 4: note = D-Alanine
PGYAGP

SEQ ID NO: 55 - protein, 7 aa, synthetic construct
AQGVAPG

SEQ ID NO: 56 - protein, 7 aa, synthetic construct
  SITE 3: note = D-Alanine
  SITE 7: note = D-Alanine
  SITE 2: note = D-Proline
  SITE 4: note = D-Valine
  SITE 6: note = D-Glutamine
GPAVGQA

SEQ ID NO: 57 - protein, 7 aa, synthetic construct
LQGVAPG

SEQ ID NO: 58 - protein, 7 aa, synthetic construct
  SITE 6: note = D-Glutamine
  SITE 2: note = D-Proline
  SITE 3: note = D-Alanine
  SITE 4: note = D-Valine
  SITE 7: note = D-Leucine
GPAVGQL

SEQ ID NO: 59 - protein, 6 aa, synthetic construct
  SITE 4: note = D-Valine
  SITE 6: note = D-Valine
  SITE 2: note = D-Proline
  SITE 3: note = D-Alanine
GPAVGV

SEQ ID NO: 60 - protein, 6 aa, synthetic construct
PGAVPG

SEQ ID NO: 61 - protein, 6 aa, synthetic construct
  SITE 4: note = D-Alanine
  SITE 3: note = D-Valine
  SITE 2: note = D-Proline
  SITE 6: note = D-Proline
GPVAGP

SEQ ID NO: 62 - protein, 6 aa, synthetic construct
LGGGPG

SEQ ID NO: 63 - protein, 6 aa, synthetic construct
  SITE 2: note = D-Proline
  SITE 6: note = D-Leucine
GPGGGL

SEQ ID NO: 64 - protein, 7 aa, synthetic construct
AQGVAPG

SEQ ID NO: 65 - protein, 7 aa, synthetic construct
  SITE 2: note = D-Proline
  SITE 3: note = D-Alanine
  SITE 7: note = D-Alanine
  SITE 4: note = D-Valine
  SITE 6: note = D-Glutamine
GPAVGQA

SEQ ID NO: 66 - protein, 7 aa, synthetic construct
LQGVAPG

SEQ ID NO: 67 - protein, 7 aa, synthetic construct
  SITE 6: note = D-Glutamine
  SITE 7: note = D-Leucine
  SITE 4: note = D-Valine
  SITE 3: note = D-Alanine
  SITE 2: note = D-Proline
GPAVGQL

SEQ ID NO: 68 - protein, 6 aa, synthetic construct
PGAYPG

SEQ ID NO: 69 - protein, 6 aa, synthetic construct
  SITE 2: note = D-Proline
  SITE 6: note = D-Proline
  SITE 3: note = D-Tyrosine
  SITE 4: note = D-Alanine
GPYAGP

SEQ ID NO: 70 - protein, 6 aa, synthetic construct
  SITE 3: note = D-Alanine
  SITE 2: note = D-Proline
  SITE 4: note = D-Valine
  SITE 6: note = D-Valine
GPAVGV

SEQ ID NO: 71 - protein, 6 aa, synthetic construct
  SITE 4: note = D-Valine
  SITE 3: note = D-Alanine
  SITE 2: note = D-Proline
GPAVGV

SEQ ID NO: 72 - protein, 6 aa, synthetic construct
  SITE 2: note = D-Proline
  SITE 3: note = D-Alanine
GPAVGV

SEQ ID NO: 73 - protein, 6 aa, synthetic construct
  SITE 2: note = D-Proline
  SITE 4: note = D-Valine
GPAVGV

SEQ ID NO: 74 - protein, 6 aa, synthetic construct
  SITE 2: note = D-Proline
GPAVGV

SEQ ID NO: 75 - protein, 6 aa, synthetic construct
  SITE 2: note = D-Proline
  SITE 6: note = D-Leucine
GPGGGL

SEQ ID NO: 76 - protein, 6 aa, synthetic construct
  SITE 2: note = D-Proline
GPGGGL

SEQ ID NO: 77 - protein, 6 aa, synthetic construct
  SITE 2: note = D-Proline
  SITE 6: note = D-Proline
  SIGNAL 3: note = D-Tyrosine
  SITE 4: note = D-Alanine
GPYAGP

SEQ ID NO: 78 - protein, 6 aa, synthetic construct
  SITE 3: note = D-Alanine
  SITE 4: note = D-Tyrosine
  SITE 2: note = D-Proline
GPAYGP

SEQ ID NO: 79 - protein, 6 aa, synthetic construct
  SITE 2: note = D-Proline
  SITE 3: note = D-Alanine
GPAYGP

SEQ ID NO: 80 - protein, 6 aa, synthetic construct
  SITE 2: note = D-Proline
  SITE 4: note = D-Tyrosine
GPAYGP

SEQ ID NO: 81 - protein, 6 aa, synthetic construct
  SITE 2: note = D-Proline
GPAYGP

SEQ ID NO: 82 - protein, 4 aa, synthetic construct
GVAP

SEQ ID NO: 83 - protein, 4 aa, synthetic construct
GAVP

SEQ ID NO: 84 - protein, 6 aa, synthetic construct
PGAIPG

SEQ ID NO: 85 - protein, 5 aa, synthetic construct
GAVPG

SEQ ID NO: 86 - protein, 5 aa, synthetic construct
GVLPG

SEQ ID NO: 87 - protein, 5 aa, synthetic construct
GGVPG

SEQ ID NO: 88 - protein, 5 aa, synthetic construct
GVVPG

SEQ ID NO: 89 - protein, 6 aa, synthetic construct
VVGPGA

SEQ ID NO: 90 - protein, 6 aa, synthetic construct
LGGGPG

SEQ ID NO: 91 - protein, 6 aa, synthetic construct
QGQLPG

SEQ ID NO: 92 - protein, 6 aa, synthetic construct
PGAYPG

SEQ ID NO: 93 - protein, 6 aa, synthetic construct
QGVLPA

SEQ ID NO: 94 - protein, 8 aa, synthetic construct
  SITE 2: note = D-Alanine
  SITE 4: note = D-Proline
  SITE 8: note = D-Leucine
GAGPGGGL

SEQ ID NO: 95 - protein, 8 aa, synthetic construct
  SITE 8: note = D-Leucine
  SITE 2: note = D-Alanine
  SITE 7: note = D-Alanine
  SITE 4: note = D-Proline
GAGPGGAL

SEQ ID NO: 96 - protein, 6 aa, synthetic construct
  SITE 3: note = D-Proline
  SITE 6: note = D-Leucine
  SITE 1: note = D-Alanine
AGPGGL

SEQ ID NO: 97 - protein, 7 aa, synthetic construct
  SITE 7: note = D-Alanine
  SITE 2: note = D-Proline
  SITE 6: note = D-Proline
GPGGGPA

SEQ ID NO: 98 - protein, 6 aa, synthetic construct
  SITE 2: note = D-Proline
  SITE 5: note = D-Alanine
  SITE 6: note = D-Leucine
GPGGAL

SEQ ID NO: 99 - protein, 5 aa, synthetic construct
  SITE 2: note = D-Proline
GPGGG

SEQ ID NO: 100 - protein, 5 aa, synthetic construct
  SITE 2: note = D-Proline
  SITE 4: note = D-Alanine
GPGAG

SEQ ID NO: 101 - protein, 6 aa, synthetic construct
  SITE 4: note = D-Alanine
  SITE 2: note = D-Proline
  SITE 6: note = D-Proline
GPVAGP

SEQ ID NO: 102 - protein, 6 aa, synthetic construct
  SITE 2: note = D-Proline
  SITE 3: note = D-Alanine
  SITE 4: note = D-Valine
  SITE 6: note = D-Valine
GPAVGV

SEQ ID NO: 103 - protein, 5 aa, synthetic construct
GGGPG

SEQ ID NO: 104 - protein, 5 aa, synthetic construct
GAGPG

SEQ ID NO: 105 - protein, 14 aa, synthetic construct
VVGSPSAQDEASPL

SEQ ID NO: 106 - protein, 18 aa, synthetic construct
VGVAPGVGVAPGVGVAPG

SEQ ID NO: 107 - protein, 18 aa, synthetic construct
QVGQVELGGGPGAGSLQP

SEQ ID NO: 108 - protein, 18 aa, synthetic construct
GAYPGAPGAYPGAPAPGV

SEQ ID NO: 109 - protein, 53 aa, Homo sapiens
GLVPGVGVAPGVGVAPGVGVAPGVGLAPGVGVAPGVGVAPGVGVAPGIGPGGV

SEQ ID NO: 110 - protein, 59 aa, Macaca mulatta
GLVPGVGVAPGVGVAPGVGVAPGVGVAPGVGVAPGVGVAPGVGVAPGVGIGPGGVAGVG

SEQ ID NO: 111 - protein, 50 aa, Mus musculus
GLGPGVGGVPGGVGVGGIPGGVGVGGVPGGVGPGGVTGIGAGPGGLGGAG

SEQ ID NO: 112 - protein, 47 aa, Rattus sp.
GLGPGVGGVPGGVGVGGLPGGVGPGGVTGIGTGPGTGLVPGDLGGAG

SEQ ID NO: 113 - protein, 39 aa, Mus musculus
GAGGAGALGGLVPGAVPGALPGAVPAVPGAGGVPGAGTP

SEQ ID NO: 114 - protein, 54 aa, Rattus sp.
GAGGAGTLGGLVPGAVPGALPGAVPGALPGAVPGALPGAVPGVPGTGGVPGAGT

SEQ ID NO: 115 - protein, 12 aa, synthetic construct
VGVAPGVGVAPG

SEQ ID NO: 116 - protein, 36 aa, synthetic construct
VGVAPGVGVAPGVGVAPGVGVAPGVGVAPGVGVAPG

SEQ ID NO: 117 - protein, 6 aa, synthetic construct
PGAVPG

SEQ ID NO: 118 - protein, 6 aa, synthetic construct
PGVAPG

SEQ ID NO: 119 - protein, 6 aa, synthetic construct
VGAVPG

SEQ ID NO: 120 - protein, 4 aa, synthetic construct
AVPG

SEQ ID NO: 121 - protein, 8 aa, synthetic construct
AVPGAVPG

SEQ ID NO: 122 - protein, 12 aa, synthetic construct
AVPGAVPGAVPG

SEQ ID NO: 123 - protein, 24 aa, synthetic construct
AVPGAVPGAVPGAVPGAVPGAVPG

SEQ ID NO: 124 - protein, 8 aa, synthetic construct
PGVGLAPG

SEQ ID NO: 125 - protein, 8 aa, synthetic construct
GVGGLPGG

SEQ ID NO: 126 - protein, 8 aa, synthetic construct
GVGGIPGG

SEQ ID NO: 127 - protein, 8 aa, synthetic construct
PGVGGVPG

SEQ ID NO: 128 - protein, 6 aa, synthetic construct
VGGVPG

SEQ ID NO: 129 - protein, 4 aa, synthetic construct
ALPG

SEQ ID NO: 130 - protein, 8 aa, synthetic construct
ALPGAVPG

SEQ ID NO: 131 - protein, 8 aa, synthetic construct
AVPGALPG

SEQ ID NO: 132 - protein, 10 aa, synthetic construct
GVAPGGVAPG

SEQ ID NO: 133 - protein, 6 aa, synthetic construct
  SITE 1: note = D-Valine
VGVAPG

SEQ ID NO: 134 - protein, 6 aa, synthetic construct
  SITE 1: note = D-Valine
  SITE 3: note = D-Valine
VGVAPG

SEQ ID NO: 135 - protein, 6 aa, synthetic construct
  SITE 1: note = D-Valine
  SITE 4: note = D-Alanine
VGVAPG

SEQ ID NO: 136 - protein, 6 aa, synthetic construct
  SITE 4: note = D-Alanine
  SITE 1: note = D-Valine
  SITE 3: note = D-Valine
VGVAPG

SEQ ID NO: 137 - protein, 6 aa, synthetic construct
LGGGPG

SEQ ID NO: 138 - protein, 6 aa, synthetic construct
  SITE 1: note = D-Proline
PGAYPG

SEQ ID NO: 139 - protein, 6 aa, synthetic construct
  SITE 1: note = D-Proline
  SITE 3: note = D-Alanine
PGAYPG

SEQ ID NO: 140 - protein, 6 aa, synthetic construct
  SITE 1: note = D-Proline
  SITE 4: note = D-Tyrosine
PGAYPG

SEQ ID NO: 141 - protein, 6 aa, synthetic construct
  SITE 4: note = D-Tyrosine
  SITE 3: note = D-Alanine
  SITE 1: note = D-Proline
PGAYPG

SEQ ID NO: 142 - protein, 35 aa, synthetic construct
RREAEDLQVGQVELGGGPGAGSLQPLALEGSLQKR

SEQ ID NO: 143 - protein, 7 aa, synthetic construct
AQGVAPG

SEQ ID NO: 144 - protein, 7 aa, synthetic construct
LQGVAPG

SEQ ID NO: 145 - protein, 7 aa, synthetic construct
AQGVLPG

SEQ ID NO: 146 - protein, 7 aa, synthetic construct
LQGVLPG

SEQ ID NO: 147 - protein, 8 aa, synthetic construct
AQGVAPGQ

SEQ ID NO: 148 - protein, 8 aa, synthetic construct
LQGVAPGQ

SEQ ID NO: 149 - protein, 8 aa, synthetic construct
AQGVLPGQ

SEQ ID NO: 150 - protein, 8 aa, synthetic construct
LQGVLPGQ

SEQ ID NO: 151 - protein, 12 aa, synthetic construct
LGGGPGAGSLQP

SEQ ID NO: 152 - protein, 12 aa, synthetic construct
PGAYPGAPAPGV

SEQ ID NO: 153 - protein, 24 aa, synthetic construct
VGVAPGVGVAPGVGVAPGVGVAPG

SEQ ID NO: 154 - protein, 12 aa, synthetic construct
QVGQVELGGGPG

SEQ ID NO: 155 - protein, 12 aa, synthetic construct
GAYPGAPGAYPG

SEQ ID NO: 156 - protein, 6 aa, synthetic construct
GVAPGV

SEQ ID NO: 157 - protein, 6 aa, synthetic construct
VAPGVG

SEQ ID NO: 158 - protein, 6 aa, synthetic construct
APGVGV

SEQ ID NO: 159 - protein, 6 aa, synthetic construct
PGVGVA

SEQ ID NO: 160 - protein, 6 aa, synthetic construct
GVGVAP

SEQ ID NO: 161 - protein, 6 aa, synthetic construct
LGTIPG

SEQ ID NO: 162 - protein, 8 aa, synthetic construct
LGGGPGAG

SEQ ID NO: 163 - protein, 7 aa, synthetic construct
GGGPGAG

SEQ ID NO: 164 - protein, 4 aa, synthetic construct
GGGP

SEQ ID NO: 165 - protein, 5 aa, synthetic construct
GGGPE

SEQ ID NO: 166 - protein, 5 aa, synthetic construct
GAIPG

SEQ ID NO: 167 - protein, 5 aa, synthetic construct
GVAPG

SEQ ID NO: 168 - protein, 14 aa, synthetic construct
YTTGKLPYGYGPGG

SEQ ID NO: 169 - protein, 12 aa, synthetic construct
YGARPGVGVGIP

SEQ ID NO: 170 - protein, 9 aa, synthetic construct
PGFGAVPGA

SEQ ID NO: 171 - protein, 5 aa, synthetic construct
GVYPG

SEQ ID NO: 172 - protein, 5 aa, synthetic construct
GFGPG

SEQ ID NO: 173 - protein, 6 aa, synthetic construct
VGAMPG

SEQ ID NO: 174 - protein, 6 aa, synthetic construct
VGSLPG

SEQ ID NO: 175 - protein, 6 aa, synthetic construct
VGMAPG

SEQ ID NO: 176 - protein, 5 aa, synthetic construct
VPGVG

SEQ ID NO: 177 - protein, 5 aa, synthetic construct
IPGVG

SEQ ID NO: 178 - protein, 5 aa, synthetic construct
VGVPG

SEQ ID NO: 179 - protein, 6 aa, synthetic construct
AGAIPG

SEQ ID NO: 180 - protein, 4 aa, synthetic construct
VPGV

SEQ ID NO: 181 - protein, 6 aa, synthetic construct
LGITPG

SEQ ID NO: 182 - protein, 4 aa, synthetic construct
GDNP

SEQ ID NO: 183 - protein, 4 aa, synthetic construct
GAIP

SEQ ID NO: 184 - protein, 4 aa, synthetic construct
GKVP

SEQ ID NO: 185 - protein, 4 aa, synthetic construct
GVQP

SEQ ID NO: 186 - protein, 4 aa, synthetic construct
GVGP

SEQ ID NO: 187 - protein, 4 aa, synthetic construct
GFGP

SEQ ID NO: 188 - protein, 4 aa, synthetic construct
GGIP

SEQ ID NO: 189 - protein, 4 aa, synthetic construct
GIGP

SEQ ID NO: 190 - protein, 4 aa, synthetic construct
GAGP

SEQ ID NO: 191 - protein, 5 aa, synthetic construct
GGIPP

SEQ ID NO: 192 - protein, 4 aa, synthetic construct
GQFP

SEQ ID NO: 193 - protein, 4 aa, synthetic construct
GLSP

SEQ ID NO: 194 - protein, 5 aa, synthetic construct
GGPQP

SEQ ID NO: 195 - protein, 5 aa, synthetic construct
GPQPG

SEQ ID NO: 196 - protein, 6 aa, synthetic construct
GGPQPG

SEQ ID NO: 197 - protein, 4 aa, synthetic construct
GIPP

SEQ ID NO: 198 - protein, 5 aa, synthetic construct
GIPPA

SEQ ID NO: 199 - protein, 6 aa, synthetic construct
GGIPPA

SEQ ID NO: 200 - protein, 24 aa, synthetic construct
EAEDLQVGQVELGGGPGAGSLQPL

SEQ ID NO: 201 - protein, 20 aa, synthetic construct
DLQVGQVELGGGPGAGSLQP

SEQ ID NO: 202 - protein, 19 aa, synthetic construct
LVGQVELGGGPGAGSLQPL

SEQ ID NO: 203 - protein, 19 aa, synthetic construct
QVGQVELGGGPGAGSLQPL

SEQ ID NO: 204 - protein, 14 aa, synthetic construct
ELGGGPGAGSLQPL

SEQ ID NO: 205 - protein, 28 aa, synthetic construct
DLQVGQVELGGGPGAGSLQPLALEGSLQ

SEQ ID NO: 206 - protein, 24 aa, synthetic construct
GQVELGGGPGAGSLQPLALEGSLQ

SEQ ID NO: 207 - protein, 20 aa, synthetic construct
LGGGPGAGSLQPLALEGSLQ

SEQ ID NO: 208 - protein, 13 aa, synthetic construct
LGGGPGAGSLQPL

SEQ ID NO: 209 - protein, 13 aa, synthetic construct
LQVGQVELGGGPG

SEQ ID NO: 210 - protein, 10 aa, synthetic construct
LQVGQVELGG

SEQ ID NO: 211 - protein, 11 aa, synthetic construct
GGPGAGSLQPL

SEQ ID NO: 212 - protein, 112 aa, synthetic construct
PQGWPGAWGNQPAGAGGYPGASYPGAYPGQAPPGAYPGQAPPGAYPGAPGAYPGAPAPGVYPGPPSGPGAYPSSGQPSATGAYPATGPYGAPAGPLIVPYNLPLPGGVVPRM

SEQ ID NO: 213 - protein, 32 aa, synthetic construct
AGGYPGASYPGAYPGQAPPGAYPGQAPPGAYP

SEQ ID NO: 214 - protein, 20 aa, synthetic construct
MMRVLQAVLPPLPQVVCTYR

SEQ ID NO: 215 - protein, 43 aa, synthetic construct
GAPGAYPGAPAPGVYPGPPSGPGAYPSSGQPSATGAYPATGPY

SEQ ID NO: 216 - protein, 21 aa, synthetic construct
GAPAGPLIVPYNLPLPGGVVP

SEQ ID NO: 217 - protein, 15 aa, synthetic construct
PGAYPGQAPPGAYPG

SEQ ID NO: 218 - protein, 8 aa, synthetic construct
PGAYPGQA

SEQ ID NO: 219 - protein, 10 aa, synthetic construct
GQAPPGAYPG

SEQ ID NO: 220 - protein, 18 aa, synthetic construct
  SITE 17: note = D-Proline
  SITE 1: note = D-Valine
VGVAPGVGVAPGVGVAPG

SEQ ID NO: 221 - protein, 18 aa, synthetic construct
  SITE 1: note = D-Valine
  SITE 18: note = D-Alanine
VGVAPGVGVAPGEGSLQA

SEQ ID NO: 222 - protein, 18 aa, synthetic construct
  SITE 18: note = D-Proline
  SITE 1: note = D-Glutamine
QVGQVELGGGPGAGSLQP

SEQ ID NO: 223 - protein, 18 aa, synthetic construct
  SITE 1: note = D-Glutamine
  SITE 18: note = D-Alanine
QVGQVELGGGPGEGSLQA

SEQ ID NO: 224 - protein, 19 aa, synthetic construct
  SITE 2: note = D-Alanine
  SITE 18: note = D-Alanine
  SITE 19: note = D-Valine
GAYPGAPGAYPGEGSLQAV

SEQ ID NO: 225 - protein, 18 aa, synthetic construct
  SITE 18: note = D-Valine
  SITE 2: note = D-Alanine
GAYPGAPGAYPGAPAPGV

SEQ ID NO: 226 - protein, 18 aa, synthetic construct
  SITE 16: note = D-Alanine
  SITE 17: note = D-Proline
  SITE 1: note = D-Valine
  SITE 3: note = D-Valine
VGVAPGVGVAPGVGVAPG

SEQ ID NO: 227 - protein, 18 aa, synthetic construct
  SITE 2: note = D-Valine
  SITE 1: note = D-Glutamine
  SITE 17: note = D-Glutamine
  SITE 18: note = D-Proline
QVGQVELGGGPGAGSLQP

SEQ ID NO: 228 - protein, 18 aa, synthetic construct
  SITE 16: note = D-Proline
  SITE 18: note = D-Valine
  SITE 2: note = D-Alanine
  SITE 3: note = D-Tyrosine
GAYPGAPGAYPGAPAPGV

SEQ ID NO: 229 - protein, 18 aa, synthetic construct
  SITE 1: note = D-Valine
  SITE 3: note = D-Valine
  SITE 15: note = D-Valine
  SITE 4: note = D-Alanine
  SITE 16: note = D-Alanine
  SITE 17: note = D-Proline
VGVAPGVGVAPGVGVAPG

SEQ ID NO: 230 - protein, 18 aa, synthetic construct
  SITE 18: note = D-Proline
  SITE 1: note = D-Glutamine
  SITE 4: note = D-Glutamine
  SITE 17: note = D-Glutamine
  SITE 2: note = D-Valine
  SITE 16: note = D-Leucine
QVGQVELGGGPGAGSLQP

SEQ ID NO: 231 - protein, 18 aa, synthetic construct
  SITE 2: note = D-Alanine
  SITE 15: note = D-Alanine
  SITE 3: note = D-Tyrosine
  SITE 4: note = D-Proline
  SITE 16: note = D-Proline
GAYPGAPGAYPGAPAPGV

SEQ ID NO: 232 - protein, 18 aa, synthetic construct
  SITE 2: note = D-Proline
  SITE 8: note = D-Proline
  SITE 14: note = D-Proline
  SITE 3: note = D-Alanine
  SITE 9: note = D-Alanine
  SITE 15: note = D-Alanine
  SITE 4: note = D-Valine
  SITE 6: note = D-Valine
  SITE 10: note = D-Valine
  SITE 12: note = D-Valine
  SITE 16: note = D-Valine
  SITE 18: note = D-Valine
GPAVGVGPAVGVGPAVGV

SEQ ID NO: 233 - protein, 18 aa, synthetic construct
  SITE 17: note = D-Valine
  SITE 14: note = D-Valine
  SITE 4: note = D-Serine
  SITE 1: note = D-Proline
  SITE 8: note = D-Proline
  SITE 6: note = D-Alanine
  SITE 2: note = D-Glutamine
  SITE 15: note = D-Glutamine
  SITE 18: note = D-Glutamine
  SITE 3: note = D-Leucine
  SITE 12: note = D-Leucine
  SITE 13: note = D-Glutamic acid
PQLSGAGPGGGLEVQGVQ

SEQ ID NO: 234 - protein, 18 aa, synthetic construct
  SITE 1: note = D-Valine
  SITE 3: note = D-Proline
  SITE 5: note = D-Proline
  SITE 8: note = D-Proline
  SITE 12: note = D-Proline
  SITE 15: note = D-Proline
  SITE 4: note = D-Alanine
  SITE 6: note = D-Alanine
  SITE 10: note = D-Alanine
  SITE 13: note = D-Alanine
  SITE 17: note = D-Alanine
  SITE 9: note = D-Tyrosine
  SITE 16: note = D-Tyrosine
  SITE 18: note = D-Tyrosine
VGPAPAGPYAGPAGPYAY

SEQ ID NO: 235 - protein, 18 aa, synthetic construct
  SITE 9: note = D-Alanine
  SITE 16: note = D-Valine
  SITE 8: note = D-Proline
  SITE 14: note = D-Proline
  SITE 18: note = D-Valine
  SITE 1: note = D-Alanine
  SITE 10: note = D-Valine
  SITE 12: note = D-Valine
  SITE 15: note = D-Alanine
  SITE 2: note = D-Glutamine
  SITE 3: note = D-Leucine
  SITE 4: note = D-Serine
  SITE 6: note = D-Glutamic acid
AQLSGEGPAVGVGPAVGV

SEQ ID NO: 236 - protein, 18 aa, synthetic construct
  SITE 6: note = D-Glutamic acid
  SITE 13: note = D-Glutamic acid
  SITE 1: note = D-Alanine
  SITE 15: note = D-Glutamine
  SITE 18: note = D-Glutamine
  SITE 3: note = D-Leucine
  SITE 12: note = D-Leucine
  SITE 4: note = D-Serine
  SITE 8: note = D-Proline
  SITE 14: note = D-Valine
  SITE 17: note = D-Valine
  SITE 2: note = D-Glutamine
AQLSGEGPGGGLEVQGVQ

SEQ ID NO: 237 - protein, 18 aa, synthetic construct
  SITE 1: note = D-Alanine
  SITE 10: note = D-Alanine
  SITE 13: note = D-Alanine
  SITE 17: note = D-Alanine
  SITE 2: note = D-Glutamine
  SITE 3: note = D-Leucine
  SITE 4: note = D-Serine
  SITE 6: note = D-Glutamic acid
  SITE 9: note = D-Tyrosine
  SITE 16: note = D-Tyrosine
  SITE 18: note = D-Tyrosine
  SITE 8: note = D-Proline
  SITE 12: note = D-Proline
  SITE 15: note = D-Proline
AQLSGEGPYAGPAGPYAY

SEQ ID NO: 238 - protein, 12 aa, synthetic construct
  SITE 1: note = D-Valine
  SITE 11: note = D-Proline
VGVAPGVGVAPG

SEQ ID NO: 239 - protein, 12 aa, synthetic construct
  SITE 1: note = D-Valine
  SITE 12: note = D-Alanine
VGVAPGEGSLQA

SEQ ID NO: 240 - protein, 12 aa, synthetic construct
  SITE 1: note = D-Glutamine
  SITE 12: note = D-Proline
QGGGPGAGSLQP

SEQ ID NO: 241 - protein, 12 aa, synthetic construct
  SITE 1: note = D-Glutamine
  SITE 12: note = D-Alanine
QGGGPGEGSLQA

SEQ ID NO: 242 - protein, 13 aa, synthetic construct
  SITE 2: note = D-Alanine
  SITE 12: note = D-Alanine
  SITE 13: note = D-Valine
GAAYPGEGSLQAV

SEQ ID NO: 243 - protein, 12 aa, synthetic construct
  SITE 12: note = D-Valine
  SITE 2: note = D-Alanine
GAYPGAAPAPGV

SEQ ID NO: 244 - protein, 12 aa, synthetic construct
  SITE 10: note = D-Alanine
  SITE 11: note = D-Proline
  SITE 1: note = D-Valine
  SITE 3: note = D-Valine
VGVAPGVGVAPG

SEQ ID NO: 245 - protein, 13 aa, synthetic construct
  SITE 2: note = D-Valine
  SITE 1: note = D-Glutamine
  SITE 12: note = D-Glutamine
  SITE 13: note = D-Proline
QVGGGPGAGSLQP

SEQ ID NO: 246 - protein, 12 aa, synthetic construct
  SITE 10: note = D-Proline
  SITE 12: note = D-Valine
  SITE 2: note = D-Alanine
  SITE 3: note = D-Tyrosine
GAYPGAAPAPGV

SEQ ID NO: 247 - protein, 12 aa, synthetic construct
  SITE 1: note = D-Valine
  SITE 3: note = D-Valine
  SITE 9: note = D-Valine
  SITE 4: note = D-Alanine
  SITE 10: note = D-Alanine
  SITE 11: note = D-Proline
VGVAPGVGVAPG

SEQ ID NO: 248 - protein, 14 aa, synthetic construct
  SITE 14: note = D-Proline
  SITE 1: note = D-Glutamine
  SITE 4: note = D-Glutamine
  SITE 13: note = D-Glutamine
  SITE 2: note = D-Valine
QVGQVELGGGPGQP

SEQ ID NO: 249 - protein, 12 aa, synthetic construct
  SITE 2: note = D-Alanine
  SITE 9: note = D-Alanine
  SITE 3: note = D-Tyrosine
  SITE 4: note = D-Proline
  SITE 10: note = D-Proline
GAYPGAAPAPGV

SEQ ID NO: 250 - protein, 12 aa, synthetic construct
  SITE 2: note = D-Proline
  SITE 8: note = D-Proline
  SITE 3: note = D-Alanine
  SITE 9: note = D-Alanine
  SITE 4: note = D-Valine
  SITE 6: note = D-Valine
  SITE 10: note = D-Valine
  SITE 12: note = D-Valine
GPAVGVGPAVGV

SEQ ID NO: 251 - protein, 14 aa, synthetic construct
  SITE 13: note = D-Valine
  SITE 1: note = D-Proline
  SITE 8: note = D-Proline
  SITE 2: note = D-Glutamine
  SITE 14: note = D-Glutamine
  SITE 3: note = D-Leucine
  SITE 12: note = D-Leucine
  SITE 4: note = D-Serine
  SITE 6: note = D-Glutamic acid
PQLSGEGPGGGLVQ

SEQ ID NO: 252 - protein, 13 aa, synthetic construct
  SITE 1: note = D-Valine
  SITE 3: note = D-Proline
  SITE 5: note = D-Proline
  SITE 7: note = D-Proline
  SITE 10: note = D-Proline
  SITE 4: note = D-Alanine
  SITE 8: note = D-Alanine
  SITE 12: note = D-Alanine
  SITE 11: note = D-Tyrosine
  SITE 13: note = D-Tyrosine
VGPAPGPAGPYAY

SEQ ID NO: 253 - protein, 12 aa, synthetic construct
  SITE 8: note = D-Proline
  SITE 1: note = D-Alanine
  SITE 9: note = D-Alanine
  SITE 2: note = D-Glutamine
  SITE 3: note = D-Leucine
  SITE 4: note = D-Serine
  SITE 6: note = D-Glutamic acid
  SITE 10: note = D-Valine
  SITE 12: note = D-Valine
AQLSGEGPAVGV

SEQ ID NO: 254 - protein, 14 aa, synthetic construct
  SITE 1: note = D-Alanine
  SITE 2: note = D-Glutamine
  SITE 11: note = D-Glutamine
  SITE 14: note = D-Glutamine
  SITE 3: note = D-Leucine
  SITE 4: note = D-Serine
  SITE 6: note = D-Glutamic acid
  SITE 8: note = D-Proline
  SITE 13: note = D-Valine
AQLSGEGPGGQGVQ

SEQ ID NO: 255 - protein, 14 aa, synthetic construct
  SITE 1: note = D-Alanine
  SITE 9: note = D-Alanine
  SITE 13: note = D-Alanine
  SITE 2: note = D-Glutamine
  SITE 3: note = D-Leucine
  SITE 4: note = D-Serine
  SITE 6: note = D-Glutamic acid
  SITE 8: note = D-Proline
  SITE 11: note = D-Proline
  SITE 12: note = D-Tyrosine
  SITE 14: note = D-Tyrosine
AQLSGEGPAGPYAY

SEQ ID NO: 256 - protein, 20 aa, synthetic construct
MTRVLQGVLPALPQVVCNYR

SEQ ID NO: 257 - protein, 4 aa, synthetic construct
LQGV

SEQ ID NO: 258 - protein, 6 aa, synthetic construct
VLPALP

SEQ ID NO: 259 - protein, 4 aa, synthetic construct
AQGV

SEQ ID NO: 260 - protein, 4 aa, synthetic construct
LAGV

SEQ ID NO: 261 - protein, 4 aa, synthetic construct
LQAV

SEQ ID NO: 262 - protein, 4 aa, synthetic construct
LQGA

SEQ ID NO: 263 - protein, 6 aa, synthetic construct
ALPALP

SEQ ID NO: 264 - protein, 6 aa, synthetic construct
VAPALP

SEQ ID NO: 265 - protein, 6 aa, synthetic construct
VLAALP

SEQ ID NO: 266 - protein, 6 aa, synthetic construct
VLPAAP

SEQ ID NO: 267 - protein, 6 aa, synthetic construct
VLPALA

SEQ ID NO: 268 - protein, 13 aa, synthetic construct
VLQGVLPALPQVV

SEQ ID NO: 269 - protein, 11 aa, synthetic construct
AGGYPGASYPG

SEQ ID NO: 270 - protein, 10 aa, synthetic construct
GAPGAYPGAP

SEQ ID NO: 271 - protein, 10 aa, synthetic construct
GAPAGPLIVP

SEQ ID NO: 272 - protein, 13 aa, synthetic construct
VLQAVLPPLPQVV

SEQ ID NO: 273 - protein, 7 aa, synthetic construct
VLQAVLP

SEQ ID NO: 274 - protein, 4 aa, synthetic construct
VLQA

SEQ ID NO: 275 - protein, 6 aa, synthetic construct
LQAVLP

SEQ ID NO: 276 - protein, 6 aa, synthetic construct
PLPQVV

SEQ ID NO: 277 - protein, 5 aa, synthetic construct
PPLQV

SEQ ID NO: 278 - protein, 16 aa, synthetic construct
RRAQGVPQIAVLVTHR

SEQ ID NO: 279 - protein, 11 aa, synthetic construct
AQGVPQIAVLV

SEQ ID NO: 280 - protein, 6 aa, synthetic construct
AQGVPQ

SEQ ID NO: 281 - protein, 7 aa, synthetic construct
AQGVPQI

SEQ ID NO: 282 - protein, 8 aa, synthetic construct
AQGVPQIA

SEQ ID NO: 283 - protein, 9 aa, synthetic construct
GVPQIAVLV

SEQ ID NO: 284 - protein, 7 aa, synthetic construct
PQIAVLV

SEQ ID NO: 285 - protein, 5 aa, synthetic construct
IAVLV

SEQ ID NO: 286 - protein, 181 aa, Homo sapiens
RGAPGQYGEKGFPGDPGNPGQNNNIKGQKGSKGEQGRQGRSGQKGVQGSPSSRGSRGREGQRGLRGVSGEPGNPGPTGTLGAEGLQGPQGSQGNPGRKGEKGSQGQKGPQGSPGLMGAKGSTGRPGLLGKKGEPGLPGDLGPVGQTGQRGRQGDSGIPGYGQMGRKGVKGPRGFPGDAGQK

SEQ ID NO: 287 - protein, 15 aa, synthetic construct
KDLCQAQGVALQTMK

SEQ ID NO: 288 - protein, 8 aa, synthetic construct
QAQGVALQ

SEQ ID NO: 289 - protein, 5 aa, synthetic construct
AQGVA

SEQ ID NO: 290 - protein, 6 aa, synthetic construct
AQGVAL

SEQ ID NO: 291 - protein, 6 aa, synthetic construct
QGVALQ

SEQ ID NO: 292 - protein, 5 aa, synthetic construct
GVALQ

SEQ ID NO: 293 - protein, 7 aa, synthetic construct
  SITE 2: note = D-Proline
  SITE 3: note = D-Alanine
  SITE 7: note = D-Alanine
  SITE 4: note = D-Valine
  SITE 6: note = D-Glutamine
GPAVGQA

SEQ ID NO: 294 - protein, 7 aa, synthetic construct
  SITE 6: note = D-Glutamine
  SITE 2: note = D-Proline
  SITE 4: note = D-Valine
  SITE 7: note = D-Leucine
  SITE 3: note = D-Alanine
GPAVGQL

SEQ ID NO: 295 - protein, 6 aa, synthetic construct
  SITE 2: note = D-Proline
  SITE 3: note = D-Alanine
  SITE 4: note = D-Valine
GPAVGV

SEQ ID NO: 296 - protein, 6 aa, synthetic construct
  SITE 2: note = D-Proline
  SITE 3: note = D-Alanine
GPAVGV

SEQ ID NO: 297 - protein, 6 aa, synthetic construct
  SITE 2: note = D-Proline
  SITE 4: note = D-Valine
GPAVGV

SEQ ID NO: 298 - protein, 6 aa, synthetic construct
  SITE 2: note = D-Proline
GPAVGV

SEQ ID NO: 299 - protein, 6 aa, synthetic construct
  SITE 2: note = D-Proline
  SITE 6: note = D-Leucine
GPGGGL

SEQ ID NO: 300 - protein, 6 aa, synthetic construct
  SITE 2: note = D-Proline
GPGGGL

SEQ ID NO: 301 - protein, 6 aa, synthetic construct
  SITE 2: note = D-Proline
  SITE 3: note = D-Tyrosine
  SITE 4: note = D-Alanine
  SITE 6: note = D-Proline
GPYAGP

SEQ ID NO: 302 - protein, 6 aa, synthetic construct
  SITE 2: note = D-Proline
  SITE 3: note = D-Alanine
  SITE 4: note = D-Tyrosine
GPAYGP

SEQ ID NO: 303 - protein, 6 aa, synthetic construct
  SITE 2: note = D-Proline
  SITE 3: note = D-Alanine
GPAYGP

SEQ ID NO: 304 - protein, 6 aa, synthetic construct
  SITE 2: note = D-Proline
  SITE 4: note = D-Tyrosine
GPAYGP

SEQ ID NO: 305 - protein, 6 aa, synthetic construct
  SITE 2: note = D-Proline
GPAYGP

SEQ ID NO: 306 - protein, 5 aa, synthetic construct
EGSLQ

SEQ ID NO: 307 - protein, 5 aa, synthetic construct
EVPPQ

SEQ ID NO: 308 - protein, 5 aa, synthetic construct
EGALQ

SEQ ID NO: 309 - protein, 5 aa, synthetic construct
EGPLQ

SEQ ID NO: 310 - protein, 5 aa, synthetic construct
EVAQQ

SEQ ID NO: 311 - protein, 5 aa, synthetic construct
EGPPQ

SEQ ID NO: 312 - protein, 5 aa, synthetic construct
EAPLQ

SEQ ID NO: 313 - protein, 5 aa, synthetic construct
EMALQ

SEQ ID NO: 314 - protein, 5 aa, synthetic construct
EVARQ

SEQ ID NO: 315 - protein, 5 aa, synthetic construct
  SITE 1: note = D-Glutamine
  SITE 2: note = D-Leucine
  SITE 3: note = D-Serine
  SITE 5: note = D-Glutamic acid
QLSGE

SEQ ID NO: 316 - protein, 5 aa, synthetic construct
  SITE 5: note = D-Glutamic acid
  SITE 4: note = D-Valine
  SITE 3: note = D-Proline
  SITE 2: note = D-Proline
  SITE 1: note = D-Glutamine
QPPVE

SEQ ID NO: 317 - protein, 5 aa, synthetic construct
  SITE 1: note = D-Glutamine
  SITE 2: note = D-Leucine
  SITE 3: note = D-Alanine
  SITE 5: note = D-Glutamic acid
QLAGE

SEQ ID NO: 318 - protein, 5 aa, synthetic construct
  SITE 5: note = D-Glutamic acid
  SITE 3: note = D-Proline
  SITE 2: note = D-Leucine
  SITE 1: note = D-Glutamic acid
QLPGE

SEQ ID NO: 319 - protein, 5 aa, synthetic construct
  SITE 5: note = D-Glutamic acid
  SITE 4: note = D-Valine
  SITE 3: note = D-Alanine
  SITE 2: note = D-Glutamine
  SITE 1: note = D-Glutamine
QQAVE

SEQ ID NO: 320 - protein, 5 aa, synthetic construct
  SITE 1: note = D-Glutamine
  SITE 2: note = D-Proline
  SITE 3: note = D-Proline
  SITE 5: note = D-Glutamic acid
QPPGE

SEQ ID NO: 321 - protein, 5 aa, synthetic construct
  SITE 5: note = D-Glutamic acid
  SITE 4: note = D-Alanine
  SITE 3: note = D-Proline
  SITE 2: note = D-Leucine
  SITE 1: note = D-Glutamine
QLPAE

SEQ ID NO: 322 - protein, 5 aa, synthetic construct
  SITE 1: note = D-Glutamine
  SITE 2: note = D-Leucine
  SITE 3: note = D-Alanine
  SITE 4: note = D-Methionine
  SITE 5: note = D-Glutamic acid
QLAME

SEQ ID NO: 323 - protein, 5 aa, synthetic construct
  SITE 5: note = D-Glutamic acid
  SITE 4: note = D-Valine
  SITE 3: note = D-Alanine
  SITE 2: note = D-Arginine
  SITE 1: note = D-Glutamine
QRAVE

SEQ ID NO: 324 - protein, 9 aa, synthetic construct
ELGGGPEAG

SEQ ID NO: 325 - protein, 5 aa, synthetic construct
EGSLQ

SEQ ID NO: 326 - protein, 31 aa, Homo sapiens
EAEDLQVGQVELGGGPGAGSLQPLALEGSLQ

SEQ ID NO: 327 - protein, 29 aa, Mus musculus
EVEDPQVEQLELGGSPGDLQTLALEVARQ

SEQ ID NO: 328 - protein, 31 aa, Mus musculus
EVEDPQVAQLELGGGPGAGDLQTLALEVAQQ

SEQ ID NO: 329 - protein, 31 aa, Rattus sp.
EVEDPQVPQLELGGGPEAGDLQTLALEVARQ

SEQ ID NO: 330 - protein, 31 aa, Rattus sp.
EVEDPQVAQLELGGGPGAGDLQTLALEVARQ